(19) United States
(12) Patent Application Publication
(10) Pub. No.: US 2025/0101521 A1
(43) Pub. Date: Mar. 27, 2025

(54) EXHALED MICRORNAS FOR THE DETECTION OF HUMAN LUNG PATHOLOGY

(71) Applicants: Albert Einstein College of Medicine, Bronx, NY (US); Hackensack Meridian Health Center for Discovery and Innovation, Nutley, NJ (US)

(72) Inventors: Simon D. Spivack, Sleepy Hollow, NY (US); Miao Kevin Shi, Great Neck, NY (US); Olivier Loudig, Yorktown Heights, NY (US); Megan Mitchell, Clifton, NJ (US)

(21) Appl. No.: 18/975,737

(22) Filed: Dec. 10, 2024

Related U.S. Application Data
(63) Continuation of application No. PCT/US23/25045, filed on Jun. 12, 2023.
(60) Provisional application No. 63/350,933, filed on Jun. 10, 2022.

(51) Int. Cl. C12Q 1/6883

(52) U.S. Cl. CPC C12Q 1/6883; C12Q 2600/106; C12Q 2600/118; C12Q 2600/178

(57) ABSTRACT

Provided herein are compositions and methods for detecting exhaled microRNAs for identifying lung diseases such as cancer, asthma, and chronic obstructive pulmonary disease.

### CROSS-REFERENCE TO RELATED APPLICATIONS

This application claims the benefit of U.S. Provisional Application No. 63/350,933, filed on Jun. 10, 2022, the entire contents of which are incorporated herein in their entirety by this reference.

### STATEMENT OF RIGHTS

This invention was made with government support under R33HL156279, 1 R21 CA192168-01, P30CA013330, and K24CA139054 awarded by the National Institutes of Health; and LCRP Expansion Award 2016 and Concept Award 2012 awarded by the U.S. Department of Defense. The government has certain rights in the invention.

### BACKGROUND OF THE INVENTION

A non-invasive portal to the lung would be useful in many clinical and epidemiologic efforts for early disease detection. For example, early detection of lung cancer, asthma, or COPD subtypes gives patients an opportunity to thwart exacerbations. For lung cancer screening, there is a consensus that the positive predictive value, efficiency, and mortality benefit of CT or other screening modalities for early lung cancer detection could be better leveraged by defining up-front an even higher risk subpopulation to screen. Clinical risk factors alone do not adequately capture overall risk nor define the highest risk subgroups, as most of the risk for lung cancer remains unexplained by standard clinical factor-based risk profiling. Therefore, the pursuit of molecular markers of risk is pivotal to improving current lung cancer screening efforts, to focus on those individuals most likely to benefit. While blood-based markers have been suggested, assessments of the broad lung epithelial field from which lung cancer arises, for messenger RNAs in invasive bronchial brushings have been more convincing. However, non-invasive, airway-based molecular risk-assessment tools are not available for clinical use at present.

Accordingly, there is a great need for non-invasive airway-based biomarkers in lung cancer, asthma, COPD, and other lung disorders for both diagnosis and risk assessment. A non-invasive portal to detect risk, or disease activity before it is clinically manifest, would be helpful in phenotyping individuals more accurately for more targeted therapies, and in detecting and pre-empting lung cancer, severe asthma and COPD and their exacerbations, along with other lung disorders.

### SUMMARY OF THE INVENTION

The present disclosure is based, at least in part, on the discovery that exhaled microRNAs provide a non-invasive surrogate for deep lung microRNAs, and detection of the exhaled microRNAs allow diagnosis of lung cancer risk without an invasive lung biopsy.

Non-invasive access to the lung is a pre-requisite for assessing lung health or pathobiology, particularly at early stages of a disease process that are not symptomatic, and therefore not typically warranting invasive biopsy procedures. Since almost all lung diagnoses are made on the airway/epithelial side, diseases of the airway (asthma, COPD, lung carcinogenesis, and others) are particularly amenable to a non-invasive airway-based approach. The early detection of such diseases, and/or the early detection of progression or exacerbation of such diseases, is a true gap in the armamentarium for early detection and prevention of lung diseases. This non-invasive, easily procured exhaled breath condensate specimen and its proper handling in turn, poses the possibility of a major advance in early detection/monitoring and therefore prevention of many common lung diseases.

For early lung cancer detection, there are ˜90 million current and former smokers at risk yielding ˜150-200 k incident cases per year. A large fraction of those become deaths (130-150 k/year), such that lung cancer is the highest population attributable cause of cancer deaths. Because it develops over decades, there is the real possibility of detecting lung carcinogenesis, before clinically apparent and incurable. Indeed, there is a consensus that identification of a highest risk group, those undergoing early molecular carcinogenesis, would leverage the current modest impact of CT screening for early lung cancer detection, and also point to a group upon whom to focus cancer prevention strategies. This pre-emptive strategy also holds for asthma, COPD, and other diseases, such that poor control or brewing exacerbations could be pre-empted. Even for stable disease, it is envisioned that phenotypic subtypes could allow more targeted therapies.

The present disclosure describes an exhaled microRNA approach to non-invasive interrogation of the lung for the purpose of developing a lung cancer risk biomarker that is both airway compartment-derived, and population-applicable. As a first step, a candidate microRNA pool was derived, including a total of 20 upregulated microRNAs that differentiated frank human lung NSCLC tumors versus paired non-tumor tissue from the same individual surgical resections, using RNAseq efforts from sample sets (FIG. 1), and verified them with analogous data from the The Cancer Genome Anatomy project, NCBI/NCI/NIH (hereinafter TCGA). Five additional candidate microRNAs were also added. Next the technical feasibility of detecting microRNAs in exhaled breath condensate (EBC) was tested, and then assessed the initial exhaled microRNA performance in discriminating those with non-small cell lung cancer (cases), and those without lung cancer (controls), each drawn from the same clinical population of individuals destined for bronchoscopy or lung resection surgery. Starting with a robust base clinical model in the discovery set, for the three primary analyses (all categories, formers smokers, early stage), the exhaled microRNA biomarker data yield a 1.1-2.5% increment in case-control discrimination attributable to the addition of qualitative exhaled microRNA data, over and above clinical factor models alone (FIGS. 3-5 and 5). The technical presence of microRNA in exhaled breath condensate is unequivocally demonstrated.

Accordingly, provided herein are methods for cancer diagnosis and prognosis based on the presence and/or level of exhaled microRNAs in EBC that is indicative of lung cancers.

### BRIEF DESCRIPTION OF FIGURES

FIG. 1A-FIG. 1B show heat map showing differential tumor versus non-tumor expression of microRNAs in lung cancer. MicroRNA-seq was used to investigate differential tumor versus non-tumor expression of microRNAs in non-microdissected bulk tissues. (FIG. 1A) Differential expression of microRNAs between adenocarcinoma tumor tissues and adjacent non-tumor tissues. (FIG. 1B) Differential expression of microRNAs between squamous cell carcinoma tumor tissues and adjacent non-tumor tissues. Each was used to help develop the EBC microRNA panel.

FIG. 2A-FIG. 2B show percent composition of 10 most abundant miRNAs found in Adenocarcinoma and Squamous Cell Carcinoma of the Lung. (FIG. 2A) Percent composition of 10 most abundant miRNAs found in bulk adenocarcinoma tumor tissue and adjacent non-tumor tissue. (FIG. 2B) Percent composition of 10 most abundant miRNAs found in bulk squamous cell carcinoma tumor tissue and adjacent non-tumor tissue. Each was used to help develop the EBC microRNA panel.

FIG. 3A-FIG. 3D show Receiver Operating Curves (ROC). Compare clinical factors alone model versus clinical factors+exhaled microRNAs combined model (red), to distinguish early stage (I+II) lung cancer all tumor histologies case donors combined, versus non-cancer controls. (FIG. 3A) All smoking categories combined; (FIG. 3B), former smokers only; (FIG. 3C) early stage only. Random forests, recursive partitioning and cross-validation were employed as described in the statistical analysis section in Example 1. This ROC plot of true positives versus false positives shows borderline incremental information (FIG. 3A, Clinical AUC+1.2% (p=0.07)) value of the exhaled microRNAs over and above the clinical model alone, in all subjects combining all smoking status', stages, and histologies. It particularly held true in main subgroup analyses, separating out former smokers (FIG. 3B, Clinical AUC+3.0% (p=6.0e-03)), and in early stage (I,II) models (FIG. 3C, Clinical AUC+2.2% (5.1e-03)). In combining these subgroups (FIG. 3D), early stage x former smokers combined did not show significant case-control discrimination (FIG. 3D, NS). Model components and significance testing of area under curve (AUC) differences are described in Table 3.

FIG. 4A-FIG. 4B show current smokers only (left), cases versus non-cancer controls; Adenocarcinoma subjects only (right). Random forests, recursive partitioning and cross-validation were employed as described in the statistical analysis section. For Current smokers (left, FIG. 4A), value of the exhaled microRNAs over and above the clinical model alone, was apparent Clinical model AUC+3.3% (p=3.5e-02)). For adenocarcinoma case subsets (right, FIG. 4B), the miR model detracted from the clinical+miR combined case-control discrimination, (Clinical AUC-2.1% (p=1.1e-02). Model components and significance testing of area under curve (AUC) differences are described in Table 3.

FIG. 5A-FIG. 5B show Receiver Operating Curves (ROC) of lung cancer case donors versus non-cancer controls. (FIG. 5A) For Late stage (III+IV) lung cancer case donors versus non-cancer controls. There was no incremental information value of the exhaled microRNAs over and above the clinical model alone (Clinical AUC+1.2% (NS)). (FIG. 5B) For Late stage x Former smoker subset, there was no incremental information value of the exhaled microRNAs over and above the clinical model alone (Clinical AUC+0.8% (NS)).

FIG. 6 shows temporal stability of EBC miRNA for an individual across time. S, K, D, and SS represented different volunteers; and by example, S1, S2, and S3 represented different timepoints (0, 24, 96 hours) from the same subject(S). Not all subjects were able to provide each timepoints. Correlation coefficients are represented on right.

FIG. 7 shows optimization of EBC microRNA extraction. PCR product SYBR fluorescence is depicted on y-axis, cycle number on x-axis. Conditions: a. ethanol precipitated miRNAs; b. trizol-purified miRNAs; c. speed-vacuum concentrated miRNAs; d. column-purified miRNAs.

FIG. 8 shows optimization of nucleic acid ethanol precipitation conditions. PCR product SYBR fluorescence is depicted on y-axis, cycle number on x-axis. Conditions: a. original miRNAs without ethanol precipitation; b. ethanol precipitated miRNAs with 20 ug glycogen; c. ethanol precipitated miRNAs with 40 ug glycogen; d. ethanol precipitated miRNAs with 80 ug glycogen; e. ethanol precipitated miRNAs with 1 ug carrier RNA; f. ethanol precipitated miRNAs with 2 ug carrier RNA; g. ethanol precipitated miRNAs with 5 ug carrier RNA.

FIG. 9 shows specificity testing of any newly designed microRNA primer. PCR products were loaded in EtBr gel (Note that 40 bp URTtag+22 bp microRNA template=62 bp PCR product): lane 1:10 bp DNA ladder; lane 2: cDNA of cultured lung cell microRNA and mRNA (total RNA extract), with polyadenylation and RT steps, cultured lung cell total RNA; lane 3: No RT step, total RNA from cultured lung cells; lane 4: cDNA of mRNA (no poly-adenylation step, total RNA from cultured lung cells); lane 5: cDNA of EBC microRNA and mRNA (all steps included); lane 6: Genomic DNA spike-in as only template; lane 7: Water blank/no template control. For a microRNA-specific PCR primer set, microRNA-size PCR bands are uniquely seen in lane 2 (cultured lung cell mix RNA extract template, all polyadenylation and RT steps included), and lane 5 (EBC RNA extract template, all polyadenylation and RT steps included). PCR product size is ˜ 60-63, reflecting the 20-23 bp microRNA template, and the 40 bp URT tag integrated at the RT step

FIG. 10A-FIG. 10C show universal RT primer optimization for microRNAs specificity. Among the URT options tested, XT-UPRT, UPRT*_V and UPRT-4 were potentially acceptable URT primers. (FIG. 10A) miRNA realtime PCR melting curve plot (left) and amplification plot (right). (FIG. 10B). agarose gel of different URT miRNA realtime PCR products. Lane 1: cDNA of miRNA (XT-URT); Lane 2: cDNA of mRNA (XT-URT); Lane 3: cDNA of miRNA (UPRT*-V); Lane 4: cDNA of mRNA (UPRT*-V); Lane 5: cDNA of miRNA (UPRT-4); Lane 6: cDNA of mRNA (UPRT-4); Lane 7: gDNA (A549 and Hela cell); Lane 8: water control. (FIG. 10C) Sequence differences among XT-UPRT, UPRT*-V and UPRT-4. In general, however, microRNA-size PCR products would not be expected to appear when PCR amplifying the cDNA of mRNA. However, such microRNA-size products were seen when amplifying the cDNA of mRNA produced by XT-UPRT and UPRT*_V, and the melting curves (FIG. 10A.top, middle) and PCR fragment sizes (FIG. 10B lanes 2,4) are very similar between cDNA of microRNA and cDNA of mRNA. For UPRT-4, there was no similar melting curve and no PCR band in cDNA of mRNA.

FIG. 11 shows a schematic representation of the EV-CATCHER® assay designed for purification of small-EVs from biofluids, including targetable specifically to EVs from cells of interest, which relies on binding of a degradable dsDNA-linker (uracilated 5′-azide oligonucleotide annealed to complementary uracilated 3′-biotin oligonucleotide) to a DBCO-activated antibody and to a streptavidin-coated platform. FIG. 11 has been adapted from FIG. 2A of US2022/0205990 and WO2022/140662, each of which is incorporated herein by reference.

FIG. 12A-FIG. 12B show micro/small RNA NGS analysis of whole EBC, EBC exosomes, and 4 other airway levels from each of 18 subjects, each providing all 5 airway specimens. The exosomes in EBC were pulled down using EXO-CATCHER® as cited. (FIG. 12A) For EBC, total read count per exosomal versus whole/non-partitioned EBC samples are displayed. (FIG. 12B) Unsupervised clustering heatmap displays the microRNA separation by airway levels. It can be seen that the EBC exosomes reflect similarity to the bronchoalveolar lavage (BAL). Housekeeper microRNAs useful for quantification of exhaled microRNAs are represented by the solid horizontal (high expressions) microRNAs, highly expressed among all sample types (miR-21, let-7A, let-7f). Data are from Illumina Hi-Seq NGS platform adapted to microRNAseq.

FIG. 13 shows exhaled microRNA collection and amplification. The method used the handheld disposable Rtube, representative examples of URT-PCR melt curves for miRs 18a, 200c, 212 are shown.

FIG. 14 shows verification of URT-PCR sensitivity. The relative sensitivity of the URT-PCR strategy was tested against the commercial standard (probe-based qRT-PCR (TaqMan). For six of ten direct comparisons, URT-PCR detected transcript, but TaqMan did not; one example miR-140 is shown. (Top) Total RNA (1 ng/ul) from a mixture of epithelial lung cells pooled was interrogated by both methods; URT-qPCR (left) showed CT 27 versus 31 for TaqMan, or 2\hat{ }4=16-fold more sensitive. (Bottom) Total RNA extracted from EBC by conventional means (Methods) showed miR 140 as detectable by URT-qPCR, but absent by TaqMan).

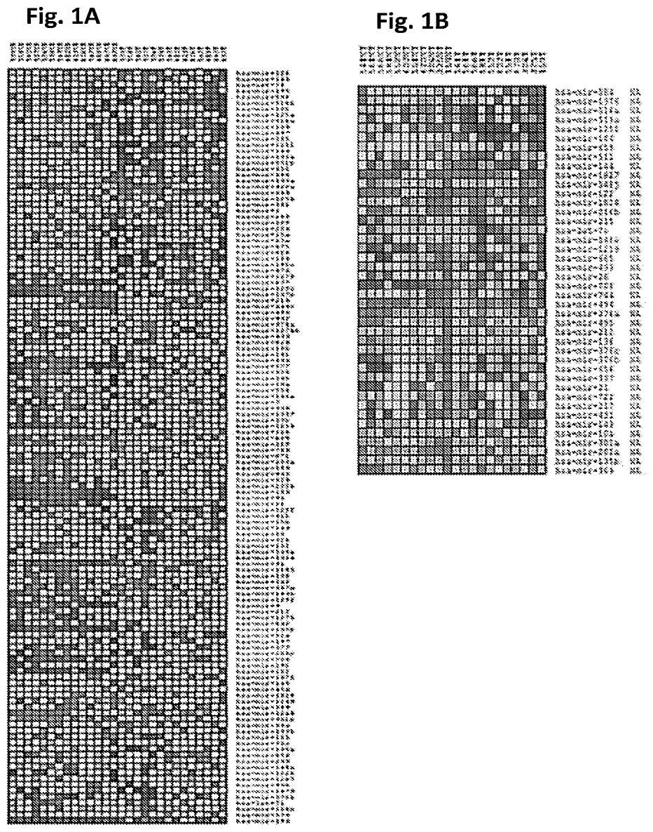

### DETAILED DESCRIPTION OF THE PRESENT INVENTION

Provided herein are methods for cancer and cancer risk, early diagnosis and/or prognosis based on the presence and/or level of exhaled microRNAs in EBC that is indicative of lung cancers. The exhaled microRNAs for asthma and COPD would be used in an analogous manner for those two diseases.

In the lung cancer early diagnostics arena, there is a consensus that the positive predictive value, efficiency, and mortality benefit of low dose CT screening modalities for early lung cancer detection, clinical diagnostics, or prevention strategies, could be better leveraged by defining up-front an even higher risk subpopulation. That is, clinical risk factors of age, smoking status, and tobacco dose, when combined into sophisticated risk models, still do not adequately capture overall risk nor define the highest risk subgroups, as most of the risk for lung cancer remains unexplained by standard clinical factor-based risk profiling. Therefore, the pursuit of molecular markers of risk is pivotal to improving current lung cancer screening and prevention efforts, to focus on those individuals most likely to benefit.

Non-invasive lung surrogates are challenging to identify. Blood does not reflect most events in the lung, particularly early molecular events, and sputum is generally not produced by most individuals. Non-invasive molecular risk-assessment tools for exhaled microRNAs have not been rigorously evaluated or quantitated, and are not in clinical use at present. By contrast, it is discovered herein that breath condensate can be a surrogate for non-invasive access to the deep lung airway. The studies presented herein indicate that EBC (e.g., EBC exosomes or EBC comprising miRNA) serves as an adequate surrogate for the deep lung (bronchial and bronchoalveolar levels).

It is described herein the development of an exhaled microRNA approach to non-invasive interrogation of the lung that is both lower airway-derived, and population-applicable, for the purpose of developing a lung cancer risk biomarker. As first steps, we confirmed the technical feasibility of detecting microRNAs in exhaled breath condensate (EBC). MicroRNA isolation from unfractionated whole EBC was optimized. Our reverse transcription (Universal-tagged RT) platform was adapted, and showed enhanced sensitivity/performance of the coupled URT-PCR platform in EBC, as compared to a standard, widely used probe-based RT-PCR platform (TaqMan, Invitrogen). MicroRNA URT and PCR steps were then further developed. A candidate microRNA pool was then generated, including upregulated microRNAs that differentiated homogenized human lung NSCLC tumors versus paired remote non-tumor tissue from the same individual surgical resections, using RNAseq from 32 surgical sample sets (ENA accession: PRJEB52036). These were then verified against analogous data from the TCGA. Using this 24-microRNA panel, exhaled microRNA-qualitative PCR performance in discriminating those with nonsmall cell lung cancer (cases), and those without lung cancer (controls) drawn from similar clinical populations of individuals destined for bronchoscopy or lung resection surgery was then assessed. Starting with a robust base clinical model, for the three primary data analyses (all clinical categories, former smokers, or early stage), the exhaled qualitative microRNA-PCR biomarker data yield a modest increment in case-control discrimination, over and above clinical factor models alone.

Accordingly, this disclosure encompasses a composite method for detecting exhaled microRNAs as a non-invasive surrogate for deep lung microRNAs, and for lung pathogenesis/diseases more generally. In some embodiments, the method: (i) entails exhaled breath condensate (EBC) collection in a manner compatible with RNA detection; (ii) may entail the pull of exosomes specific to lung; (iii) may entail sequencing of exhaled miRNAs; (iv) entails a URT-PCR [or other nucleic acid amplification technique] that is microRNA-specific.

For collection, the subject breathes quietly as tidal breathing. The exhaled breath can be diverted and condensed in a cooling chamber, using lab constructed or commercially available means. This aqueous EBC material can be analyzed as a crude unpartitioned liquid sample, or partitioned to exosomes and supernatant. In some embodiments, a technology for exosome capture where necessary, EV-CATCHER® (Mitchell et al. ((2021) J Extracell Vesicles 10: e12110; WO2022140662; and US20220205990, each of which is incorporated herein by reference), may be used. The EBC liquid sample may then be extracted for nucleic acids, optionally followed by size separation for focus on microRNA expression evaluations. The microRNA fraction (exosomal or non-exosomal) can be analyzed by next generation sequencing, PCR, or analogous strategies described herein or known in the art. Exemplary features may include EBC collection, sample partitioning/fractionation to lung-specific exosomes where needed, small/microRNA extraction, and microRNA amplification, and detection, e.g., by next generation sequencing (NGSeq).

A microRNA-seq discovery effort compared paired tumor to non-tumor tissue, was reconciled with analogous TCGA and published literature-based tissue-discriminant microRNA data, yielding a candidate panel of 24 microRNAs that are upregulated in either adenocarcinomas and/or squamous cell carcinomas. The technical feasibility of microRNA-PCR assays in exhaled breath condensate (EBC) was tested. The airway origin of exhaled microRNAs was then topographically “fingerprinted”, using paired EBC, upper and lower airway, and bronchoscopic samples. For initial EBC testing, a clinic-based case-control set of 351 individuals (166 NSCLC cases, 185 non-cancer controls) was interrogated with the 24-candidate microRNA panel by qualitative RT-PCR, and curated by melt curve analysis. Data were analyzed by both logistic regression (LR), and by random-forest (RF) models, validated by iterative resampling.

Both feasibility of exhaled microRNA detection, and its origins in part from lower airway sources, were confirmed. LR models adjusted for age, sex, smoking status, pack years, quit-years, and underlying lung disease identified exhaled miR-21, 33b, 212 (p.adj,=0.019, 0.018, 0.033, resp.) as overall case-control discriminant. For the RF analysis, the combined clinical+microRNA models showed modest added discrimination capacity (1.1-2.5%) beyond the clinical models alone: by subgroup, all subjects 1.1% (p=8.7e-04)); former smokers 2.5% (p=3.6e-05); early stage 1.2% (p=9.0e-03), yielding combined ROC AUC ranging from 0.74 to 0.83. Sensitivity, specificity, positive- and negative-predictive values of the clinical+microRNA models for the entire cohort were 71%-76%. Conclusion: This work demonstrates that exhaled microRNAs are measurable qualitatively; reflect in part lower airway signatures; and upon refinement as presented herein, can help distinguish lung cancer cases from controls and improve lung cancer risk assessment.

### Definitions

The articles “a” and “an” are used herein to refer to one or to more than one (i.e., to at least one) of the grammatical object of the article. By way of example, “an element” means one element or more than one element.

The term “preventing” is art-recognized, and when used in relation to a condition, such as a disease such as cancer is well understood in the art, and includes administration of a treatment, e.g., a composition which reduces the frequency of, or delays the onset of, symptoms of a medical condition in a subject relative to a subject which does not receive the treatment. Thus, prevention of cancer includes, for example, reducing the number of detectable cancerous growths in a population of patients receiving a prophylactic treatment relative to an untreated control population, and/or delaying the appearance of detectable cancerous growths in a treated population versus an untreated control population, e.g., by a statistically and/or clinically significant amount.

The term “remission” is art recognized, and refers to a condition in which the signs and symptoms of the cancer are reduced.

As used herein, “subject” refers to any healthy animal; mammal or human; or any animal, mammal or human afflicted with a disease such as a cancer. The term “subject” is interchangeable with “patient”. The term “non-human animal” includes all vertebrates, e.g., mammals and non-mammals, such as non-human primates, sheep, dog, cow, chickens, amphibians, reptiles, etc.

A “therapeutically effective amount” of a compound is an amount capable of producing a medically desirable result in a treated patient, e.g., decrease tumor burden, decrease the growth of tumor cells, or alleviate any symptom associated with cancer, with an acceptable benefit: risk ratio, preferably in a human or non-human mammal.

The term “treating” includes prophylactic and/or therapeutic treatments. The term “prophylactic or therapeutic” treatment is art-recognized and includes administration to the host of one or more of a cancer therapy. If it is administered prior to clinical manifestation of the unwanted condition (e.g., disease or other unwanted state of the host animal), then the treatment is prophylactic (i.e., it protects the host against developing the unwanted condition); whereas, if it is administered after manifestation of the unwanted condition, the treatment is therapeutic (i.e., it is intended to diminish, ameliorate, or stabilize the existing unwanted condition or side effects thereof).

The term “URT-PCR” refers to Universal RT-coupled PCR strategy for specific detection and accurate quantitation of RNA that is described in U.S. Pat. No. 7,141,372, which is incorporated herein by reference. See below for details.

Collecting/Analyzing/Detecting miRNA

Any methods described herein or those known in the art can be used to collect, analyze, and detect miRNAs from EBC. Such methods include, but are not limited to, RNA-seq, next generation sequencing, sequencing, mass spectrometry (e.g., RNA sequencing by LC-MS, DNA sequencing by LC-MS), microarray, Southern blotting, PCR (e.g., URT-PCR), realtime PCR (e.g., TaqMan®), testing for a differential melting temperature of a complementary DNA (cDNA) duplex of miRNA (e.g., comparing the experimentally determined melting temperature against the theoretical melting temperature that is characteristic of the miRNA sequence), any variation thereof, or any combination of two or more thereof.

### URT-PCR

URT-PCR (described in U.S. Pat. No. 7,141,372, which is incorporated herein by reference) is a PCR strategy that takes advantage of the poly-A tail of processed mRNA (or poly-A tail added to microRNA as presented herein), and uses novel “Universal RT primers” that comprise a unique 5′ tag sequence that does not occur in the genome of the organism being studied (for example the human genome), a poly-T midsection, and a 3′ anchor to avoid slippage. These 5′ tag-enhanced “Universal RT primers” reliably initiate reverse transcription, and the unique sequence of the 5′ tag is then targeted by the PCR primers. The reverse primer used for PCR can be identical to the 5′ tag of the Universal RT primer, in which case transcript specificity is conferred by the forward (sense) primer. Reverse PCR primers that are identical to the 5′ tag of the Universal RT primer are referred to as “Universal primers” (UR) or “Universal reverse primers” (URP). Genomic DNA or pseudogene amplification is avoided both by limiting reverse transcription to poly-A derived material, and by introducing a genetically engineered sequence tag that does not occur in the human genome and therefore cannot be mimicked by pseudogene sequence. The Universal RT-coupled PCR method allows for multiple different transcripts to be amplified from the same tissue derived RNA sample, across multiple experiments on the same subject, similar to oligo dT-based RT strategies.

An exemplary use of URT-PCR is also described in Example 1.

### Sequencing

Any of a variety of sequencing reactions known in the art can be used to directly sequence the miRNAs or their complementary DNA (cDNA) counterpart. Examples of sequencing reactions include those based on techniques developed by Maxam and Gilbert (1977) Proc. Natl. Acad. Sci. USA 74:560 or Sanger (1977) Proc. Natl. Acad Sci. USA 74:5463. It is also contemplated that any of a variety of automated sequencing procedures can be utilized (Naeve (1995) Biotechniques 19:448-53), including sequencing by mass spectrometry (see, e.g., PCT International Publication No. WO 94/16101; Cohen et al. (1996) Adv. Chromatogr. 36:127-162; and Griffin et al. (1993) Appl. Biochem. Biotechnol. 38:147-159). Notably, mass spectrometry (e.g., LC-MS, LC-MS/MS) may be used to sequence DNA (see Chowdhury and Guengerich (2013) Curr Protoc Nucleic Acid Chem 7: Unit-7.1611) or RNA (Zhang et al. (2019) Nucleic Acids Research 47: e125).

In certain embodiments, detection of miRNA can be accomplished using methods including, but not limited to, sequencing by hybridization (SBH), sequencing by ligation (SBL), quantitative incremental fluorescent nucleotide addition sequencing (QIFNAS), pyrosequencing, fluorescent in situ sequencing (FISSEQ), FISSEQ beads (U.S. Pat. No. 7,425,431), wobble sequencing (PCT/US05/27695), multiplex sequencing (U.S. Ser. No. 12/027,039, filed Feb. 6, 2008; Porreca et al. (2007) Nat. Methods 4:931), polymerized colony (POLONY) sequencing (U.S. Pat. Nos. 6,432,360, 6,485,944 and 6,511,803, and PCT/US05/06425); nanogrid rolling circle sequencing (ROLONY) (U.S. Ser. No. 12/120,541, filed May 14, 2008), and the like. High-throughput sequencing methods, e.g., on cyclic array sequencing using platforms such as Roche 454, Illumina Solexa or MiSeq or HiSeq, AB-SOLID, Helicos, Polonator platforms and the like, can also be utilized. High-throughput sequencing methods are described in U.S. Ser. No. 61/162,913, filed Mar. 24, 2009. A variety of light-based sequencing technologies are known in the art (Landegren et al. (1998) Genome Res. 8:769-76; Kwok (2000) Pharmocogenom. 1:95-100; and Shi (2001) Clin. Chem. 47:164-172) (see, for example, U.S. Pat. Publ. Nos. 2013/0274117, 2013/0137587, and 2011/0039304).

Next-generation sequencing (NGS) is a technology for determining the sequence of DNA or RNA to study genetic variation associated with diseases or other biological phenomena. Introduced for commercial use in 2005, this method was initially called “massively-parallel sequencing”, because it enabled the sequencing of many DNA strands at the same time, instead of one at a time as with traditional Sanger sequencing by capillary electrophoresis (CE).

Because of the speed, throughput, and accuracy of NGS, NGS enables the interrogation of hundreds to thousands of miRNAs or their cDNA counterparts at one time in multiple samples, as well as discovery and analysis of different types of genomic features in a single sequencing run, from single nucleotide variants (SNVs), to copy number and structural variants, and even RNA fusions. NGS provides the ideal throughput per run, and studies can be performed quickly and cost-effectively. Additional advantages of NGS include lower sample input requirements, higher accuracy, and ability to detect variants at lower allele frequencies than with Sanger sequencing.

Analyzing the whole genome using next-generation sequencing (NGS) delivers a base-by-base view of all genomic alterations, including single nucleotide variants (SNV), insertions and deletions, copy number changes, and structural variations. Paired-end whole-genome sequencing involves sequencing both ends of a DNA fragment, which increases the likelihood of alignment to the reference and facilitates detection of genomic rearrangements, repetitive sequences, and gene fusions.

In some embodiments, the Illumina “Phased Sequencing” platform, which employs a combination of long and short pair-ends, can be used. In other embodiments, the third-generation single-molecule sequencing technologies (e.g., ONT and PacBio) can produce much longer reads of DNA sequences.

In some embodiments, the “Deep Sequencing” or high-coverage version of Illumina NGS can be used. Deep Sequencing refers to sequencing a sample multiple times, sometimes hundreds or even thousands of times. The Deep Sequencing allows detection of miRNA, rare clonal types, cells, or microbes comprising as little as 1% of the original sample. Illumina's NovaSeq performs such whole-genome sequencing efficiently and cost-effectively, and its scalable output generates up to 6 Tb and 20 billion reads in dual flow cell mode with simple streamlined automated workflows.

### RNA-seq

RNA-seq allows for high throughput next generation sequencing (NGS), providing both qualitative and quantitative information about the different RNA species present in a given sample. There are many different types of RNA-seq. Direct RNA-seq sequences the RNA in a sample directly. This method avoids the bias introduced by complementary DNA (cDNA) synthesis, polymerase chain reaction (PCR), or adaptor ligation. However, RNA is an unstable molecule, so often RNA-seq workflows begin with conversion of RNA into CDNA.

### Microarray

In certain embodiments, detection of miRNA can be accomplished using microarrays. High-throughput microarrays have been developed to identify and detect miRNAs in a variety of samples, e.g., tissue and cell types (see, e.g., Babak et al., RNA 10:1813 (2004); Calin et al., Proc. Natl. Acad. Sci. USA 101:11755 (2004); Liu et al., Proc. Natl. Acad. Sci. USA 101:9740 (2004); Miska et al., Genome Biol. 5: R68 (2004); Sioud and Røsok, BioTechniques 37:574 (2004); Krichevsky et al., RNA 9:1274 (2003)). The use of microarrays has several advantages for detection of miRNA expression, including the ability to determine the presence and/or level of multiple miRNAs in the same sample at a single time point, a need for only small amounts of RNA, and the potential to simultaneously identify the expression of both precursor and mature miRNA molecules.

In some embodiments, covalent attachment of fluorophores can be used to directly label miRNA molecules for use in microarray analyses (see, e.g., Babak et al., RNA 10:1813 (2004); MICROMAX ASAP miRNA Chemical Labeling Kit, Perkin Elmer, Shelton, CT; Label IT® μArray Labeling Kit, Mirus Bio Corp., Madison, WI). In other embodiments, random primed-reverse transcription of miRNA molecules can be used to produce labeled cDNA molecules for use in microarray analyses (see, e.g., Sioud and Røsok, BioTechniques 37:574 (2004); Liu et al., Proc. Natl. Acad. Sci. USA 101:9740 (2004)).

### Significant Level

The “level” or “amount” of a biomarker (e.g., miRNAs) in a subject is “significantly” higher or lower than the level of a biomarker in a control (e.g., normal sample), if the amount of the biomarker is greater or less, respectively, than the level in a control by an amount greater than the standard error of the assay employed to assess amount.

In some embodiments, the amount or level of a biomarker in a subject can be considered “significantly” higher or lower than the normal and/or control amount if the amount is at least or about 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 100%, 110%, 120%, 130%, 140%, 150%, 160%, 170%, 180%, 190%, 200%, 250%, 300%, 350%, 400%, 450%, 500%, 550%, 600%, 650%, 700%, 750%, 800%, 850%, 900%, 950%, 1000%, 1500%, 2000%, 2500%, 3000%, or more, or any range in between, such as 1%-100%, higher or lower, respectively, than the normal and/or control amount of the biomarker. Such significant modulation values can be applied to any metric described herein, such as the level of miRNA.

### Exosome Partitioning

EBC can be directly analyzed for the presence and/or level of miRNAs described herein. Accordingly, in some embodiments, EBC is analyzed without partitioning the exosomes present therein. In other embodiments, EBC is processed to partition exsosomes present therein prior to analysis for the presence and/or level of miRNAs. While optional, the exosome partitioning may increase sensitivity of the detection, especially for the miRNAs whose copy number is less than 20 copies.

Any methods known in the art can be used to partition the exosomes. For example, kits that allow exosome portioning are available commercially from vendors, e.g., 1-Exosome Isolation CD63 kit from Miltenyi Biotec (Cat #130-110-918); 2-Mojosort Magnetic beads from Biolegends (Cat #480016); 3-MagCapture Tim 4 Exosome isolation kit from Fujifilm (Cat #293-77601); 4-Dynabeads MyOne TI Carboxylic Acid beads from Thermofisher (Cat #65011); 5-ExoCap CD63+ from MBL International (Cat #MEXSA123); 6-Dynabeads Streptavidin MyOne TI beads from ThermoFisher (Cat #10606D); 7-Exo-Flow32 CD63 IP exosome purification kit from System Biosciences (SBI) (Cat #EXOFLOW32A-CD63); 8-ExoEasy exosome purification kit from Qiagen (Cat #76064); 9-Plasma/serum exosome purification kit from Nörgen Biotek (Cat #57400); 10-ExoQuick purification reagent from SBI (Cat #EXOQ5TM-1). Also known in the art are traditional methods involving sedimentation of small extracellular vesicles (small EVs) using ultracentrifugation (see e.g., Lane et al. (2017) Methods Mol Biol, 1660:111-130), which is incorporated herein by reference.

In preferred embodiments, a method comprising Extracellular Vesicle Capture by AnTibody of Choice and Enzymatic Release (EV-CATCHER®) is used. The EV-CATCHER® purifies small EVs from biofluids, which relies on binding of a degradable dsDNA-linker (uracilated 5′-azide oligonucleotide annealed to complementary uracilated 3′-biotin oligonucleotide) to a DBCO-activated antibody and to a streptavidin-coated platform. Any antibody that recognizes an antigen present on small EV may be used, e.g., anti-CD63 (commercially available from e.g., Abcam, #ab59479), anti-CD81 (commercially available from, Abcam, #ab233692), or anti-CD9 (commercially available from e.g., Abcam, #ab263023). The EV-CATCHER® method is described in details in Mitchell et al. (2021) J Extracell Vesicles, 10: e12110, US2022/0205990, and WO2022/140662, each of which is incorporated herein by reference.

### Control

A control refers to any suitable reference standard, such as a normal patient, cultured primary cells/tissues isolated from a subject such as a normal subject, adjacent normal cells/tissues obtained from the same organ or body location of the patient, a tissue or cell sample isolated from a normal subject, or a primary cells/tissues obtained from a depository.

A control also refers to any reference standard suitable to provide a comparison to the expression products in the test sample. In certain embodiments, the control comprises obtaining a control sample from which the level of miRNA is detected and compared to the same from the test sample. Such a control sample may comprise any suitable sample, including but not limited to a sample from a control cancer patient (can be stored sample or previous sample measurement) with a known outcome; normal tissue or cells isolated from a subject, such as a normal patient or the cancer patient, cultured primary cells/tissues isolated from a subject such as a normal subject or the cancer patient, adjacent normal cells/tissues obtained from the same organ or body location of the cancer patient, a tissue or cell sample isolated from a normal subject, or a primary cells/tissues obtained from a depository. In some embodiments, the control may comprise a reference standard expression product (e.g., miRNA) level from any suitable source, including but not limited to an expression product level range from normal tissue (or other previously analyzed control sample), a previously determined expression product level range within a test sample from a group of patients, or a set of patients with a certain outcome (for example, survival for one, two, three, four years, etc.) or receiving a certain treatment (for example, standard of care cancer therapy). In some embodiments, the control comprises samples drawn or collected longitudinally at different times, to evaluate a change in the level of miRNA over time. It will be understood by those of skill in the art that such control samples and reference standard expression product levels can be used in combination as controls in the methods of the present disclosure.

In some embodiments, the amount of miRNA may be determined within a sample relative to, or as a ratio of, the amount of another housekeeping miRNA in the same sample. In some embodiments, the control comprises a ratio transformation of expression product levels, including but not limited to determining a ratio of product levels of two miRNAs in the test sample and comparing it to any suitable ratio of the same in a reference standard; determining product levels of the two or more miRNAs in the test sample and determining a difference in product levels in any suitable control; and determining product levels of the two or more miRNAs in the test sample, normalizing their level to the level of housekeeping gene products in the test sample, and comparing to any suitable control. In preferred embodiments, the control comprises a control sample which is of the same lineage and/or type as the test sample. In other embodiments, the control may comprise product levels grouped as percentiles within or based on a set of patient samples, such as all patients with cancer. In some embodiments, a control product level is established wherein higher or lower levels of product relative to, for instance, a particular percentile, are used as the basis for predicting outcome. In other preferred embodiments, a control product level is established using product levels from cancer control patients with a known outcome, and the product levels from the test sample are compared to the control product level as the basis for predicting outcome. As demonstrated by the data provided herein, the methods of the present disclosure are not limited to use of a specific cut-point in comparing the level of product in the test sample to the control.

In some embodiments, a pre-determined marker amount can be any suitable standard. For example, the pre-determined marker amount can be obtained from the same or a different human for whom a patient selection is being assessed. In some embodiments, the pre-determined marker amount can be obtained from a previous assessment of the same patient. In such a manner, the progress of the selection of the patient can be monitored over time. In addition, the control can be obtained from an assessment of another human or multiple humans, e.g., selected groups of humans, if the subject is a human. In such a manner, the extent of the selection of the human for whom selection is being assessed can be compared to suitable other humans, e.g., other humans who are in a similar situation to the human of interest, such as those suffering from similar or the same condition(s) and/or of the same ethnic group.

Accordingly, in some embodiments, a control comprises a sample (e.g., EBC or a derivative thereof) from a normal healthy person without cancer. In yet other preferred embodiments, a control comprises a sample (e.g., EBC or a derivative thereof) from a patient who is being evaluated (e.g., diagnosis or prognosis). For example, the control sample may comprise (i) a historical sample of the patient, or (ii) the sample obtained from the patient in longitudinal studies, e.g., pre-therapy or post-therapy (e.g., cancer therapy). The use of such control allows comparison of a biomarker present in the same patient over time (e.g., during the progression of cancer).

### Diagnostic Assays

The present disclosure provides, in part, methods, systems, and code for accurately classifying whether a biological sample comprises miRNA and/or whether the levels of miRNA are modulated (e.g., upregulated or downregulated), thereby indicative of the state of a disorder of interest, such as cancer. In some embodiments, the present invention is useful for classifying a sample (e.g., from a subject) as associated with or at risk for cancer or a subtype thereof, using a statistical algorithm and/or empirical data (e.g., the presence, absence, or level miRNA).

An exemplary method for detecting the level of miRNA, and thus useful for classifying whether a sample is associated with a cancer or a clinical subtype thereof or different stages of a cancer involves obtaining a biological sample (e.g., EBC or a derivative thereof) from a test subject and detecting miRNA in the sample using methods described herein or those known in the art.

In certain instances, the statistical algorithm is a single learning statistical classifier system. For example, a single learning statistical classifier system can be used to classify a sample as a cancer sample based upon a prediction or probability value and the presence or level of miRNA. The use of a single learning statistical classifier system typically classifies the sample as a cancer sample with a sensitivity, specificity, positive predictive value, negative predictive value, and/or overall accuracy of at least or about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99%.

Other suitable statistical algorithms are well-known to those of skill in the art. For example, learning statistical classifier systems include a machine learning algorithmic technique capable of adapting to complex data sets (e.g., panel of markers of interest) and making decisions based upon such data sets. In some embodiments, a single learning statistical classifier system such as a classification tree (e.g., random forest) is used. In other embodiments, a combination of 2, 3, 4, 5, 6, 7, 8, 9, 10, or more learning statistical classifier systems are used, preferably in tandem. Examples of learning statistical classifier systems include, but are not limited to, those using inductive learning (e.g., decision/classification trees such as random forests, classification and regression trees (C&RT), boosted trees, etc.), Probably Approximately Correct (PAC) learning, connectionist learning (e.g., neural networks (NN), artificial neural networks (ANN), neuro fuzzy networks (NFN), network structures, perceptrons such as multi-layer perceptrons, multi-layer feed-forward networks, applications of neural networks, Bayesian learning in belief networks, etc.), reinforcement learning (e.g., passive learning in a known environment such as naive learning, adaptive dynamic learning, and temporal difference learning, passive learning in an unknown environment, active learning in an unknown environment, learning action-value functions, applications of reinforcement learning, etc.), and genetic algorithms and evolutionary programming. Other learning statistical classifier systems include support vector machines (e.g., Kernel methods), multivariate adaptive regression splines (MARS), Levenberg-Marquardt algorithms, Gauss-Newton algorithms, mixtures of Gaussians, gradient descent algorithms, and learning vector quantization (LVQ). In certain embodiments, the method of the present disclosure further comprises sending the sample classification results to a clinician (a non-specialist, e.g., primary care physician; and/or a specialist, e.g., a histopathologist or an oncologist).

In some embodiments, the method of the present disclosure further provides a diagnosis in the form of a probability that the individual has a cancer. For example, the individual can have about a 0%, 5%, 10%, 15%, 20%, 25%, 30%, 35%, 40%, 45%, 50%, 55%, 60%, 65%, 70%, 75%, 80%, 85%, 90%, 95%, or greater probability of having the cancer. In yet another embodiment, the method of the present invention further provides a prognosis of the cancer in the individual. In some instances, the method of classifying a sample as a cancer sample may be further based on the symptoms (e.g., clinical factors) of the individual from which the sample is obtained. The symptoms or group of symptoms can be, for example, lymphocyte count, white cell count, erythrocyte sedimentation rate, diarrhea, abdominal pain, bloating, pelvic pain, lower back pain, cramping, fever, anemia, weight loss, anxiety, depression, and combinations thereof. In some instances, the method of classifying a sample as a cancer sample may be further based on genetic mutations and/or predisposition to cancer, irrespective of the symptoms. In some embodiments, the diagnosis of an individual as having a cancer is followed by administering to the individual a therapeutically effective amount of a cancer therapy (e.g., chemotherapeutic agents). In some embodiments, the diagnosis of an individual as having a cancer is followed by treating the individual with a cancer therapy.

In some embodiments, the methods further involve obtaining a control biological sample (e.g., biological sample from a subject who does not have a cancer), a biological sample from the subject during remission or before developing a cancer, or a biological sample from the subject during treatment for developing a cancer.

In some embodiments, the methods comprise analyzing the control sample to detect miRNA, such that the presence and/or the level of said miRNA is detected in the biological sample (e.g., EBC or a derivative thereof), and comparing the presence or the level of miRNA in the control sample with the presence or the level of miRNA in the test sample.

### Prognostic Assays

The term “prognosis” includes a prediction of the probable course and outcome of cancer (or another disease) or the likelihood of recovery from the disease. In some embodiments, the use of statistical algorithms provides a prognosis of cancer in an individual. For example, the prognosis can be surgery, development of a clinical subtype of cancer (e.g., solid tumors, such as lung cancer, melanoma, and renal cell carcinoma), development of one or more clinical factors, development of intestinal cancer, or recovery from the disease.

The assays described herein, such as the preceding diagnostic assays or the following assays, can be utilized to determine whether a subject can be administered an agent (e.g., an agonist, antagonist, peptidomimetic, polypeptide, peptide, nucleic acid, small molecule, immunotherapy, immune checkpoint inhibition therapy, or other drug candidate) to treat a cancer. For example, such methods can be used to determine whether a subject can be effectively treated with one or a combination of agents. Thus, the present disclosure provides methods for determining whether a subject can be effectively treated with one or more agents for treating a cancer in which a test sample is obtained and miRNA is detected.

Other aspects of the present disclosure include uses of the methods described herein for association and/or stratification analyses in which miRNA in biological samples from individuals with a cancer, are analyzed and the information is compared to that of controls (e.g., individuals who do not have the cancer; controls may be also referred to as “healthy” or “normal” individuals or at early timepoints in a given time lapse study) who are preferably of similar age and race. The appropriate selection of patients and controls is important to the success of association and/or stratification studies. Therefore, a pool of individuals with well-characterized phenotypes is extremely desirable. Criteria for cancer diagnosis, cancer predisposition screening, predicting clinical outcomes, cancer prognosis, determining drug responsiveness (pharmacogenomics), drug toxicity screening, etc. are described herein.

Different study designs may be used for genetic association and/or stratification studies (Modern Epidemiology, Lippincott Williams & Wilkins (1998), 609-622). Observational studies are most frequently carried out in which the response of the patients is not interfered with. The first type of observational study identifies a sample of persons in whom the suspected cause of the disease is present and another sample of persons in whom the suspected cause is absent, and then the frequency of development of disease in the two samples is compared. These sampled populations are called cohorts, and the study is a prospective study. The other type of observational study is case-control or a retrospective study. In typical case-control studies, samples are collected from individuals with the phenotype of interest (cases) such as certain manifestations of a disease, and from individuals without the phenotype (controls) in a population (target population) that conclusions are to be drawn from. Then the possible causes of the disease are investigated retrospectively. As the time and costs of collecting samples in case-control studies are considerably less than those for prospective studies, case-control studies are the more commonly used study design in genetic association studies, at least during the exploration and discovery stage.

After all relevant phenotypic and/or genotypic information has been obtained, statistical analyses are carried out to determine if there is any significant correlation between the presence of an allele or a genotype with the phenotypic characteristics of an individual. Preferably, data inspection and cleaning are first performed before carrying out statistical tests for genetic association. Epidemiological and clinical data of the samples can be summarized by descriptive statistics with tables and graphs well-known in the art. Data validation is preferably performed to check for data completion, inconsistent entries, and outliers. Chi-squared tests and t-tests (Wilcoxon rank-sum tests if distributions are not normal) may then be used to check for significant differences between cases and controls for discrete and continuous variables, respectively.

An important decision in the performance of genetic association tests is the determination of the significance level at which significant association can be declared when the p-value of the tests reaches that level. In an exploratory analysis where positive hits will be followed up in subsequent confirmatory testing, an unadjusted p-value <0.2 (a significance level on the lenient side), for example, may be used for generating hypotheses for significant association of a miRNA level with certain phenotypic characteristics of a cancer. It is preferred that a p-value <0.05 (a significance level traditionally used in the art) is achieved in order for the level to be considered to have an association with a cancer. When hits are followed up in confirmatory analyses in more samples of the same source or in different samples from different sources, adjustment for multiple testing will be performed as to avoid excess number of hits while maintaining the experiment-wise error rates at 0.05. While there are different methods to adjust for multiple testing to control for different kinds of error rates, a commonly used but rather conservative method is Bonferroni correction to control the experiment-wise or family-wise error rate (Multiple comparisons and multiple tests, Westfall et al, SAS Institute (1999)). Permutation tests to control for the false discovery rates, FDR, can be more powerful (Benjamini and Hochberg, Journal of the Royal Statistical Society, Series B 57, 1289-1300, 1995, Resampling-based Multiple Testing, Westfall and Young, Wiley (1993)). Such methods to control for multiplicity would be preferred when the tests are dependent and controlling for false discovery rates is sufficient as opposed to controlling for the experiment-wise error rates.

Once individual risk factors, genetic or non-genetic, have been found for the predisposition to disease, a classification/prediction scheme can be set up to predict the category (for instance, disease or no-disease) that an individual will be in depending on his phenotype and/or genotype and other non-genetic risk factors. Logistic regression for discrete trait and linear regression for continuous trait are standard techniques for such tasks (Applied Regression Analysis, Draper and Smith, Wiley (1998)). Moreover, other techniques can also be used for setting up classification. Such techniques include, but are not limited to, MART, CART, neural network, and discriminant analyses that are suitable for use in comparing the performance of different methods (The Elements of Statistical Learning, Hastie, Tibshirani & Friedman, Springer (2002)).

### Sample (EBC or a Derivative Thereof)

Biological samples can be collected from a variety of sources from a subject. In preferred embodiments, the biological samples are collected from a breath or a breathing condensate of a subject. For example, exhaled breathing condensate (EBC) may be used. Alternatively, the EBC may be further processed to prepare a derivative of EBC. As used herein, a reference to sample includes EBC or a derivative thereof.

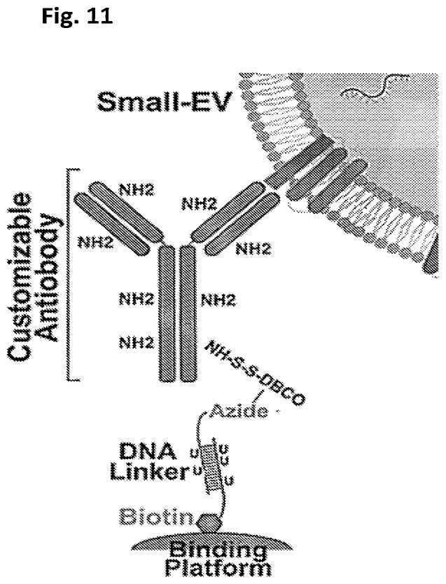

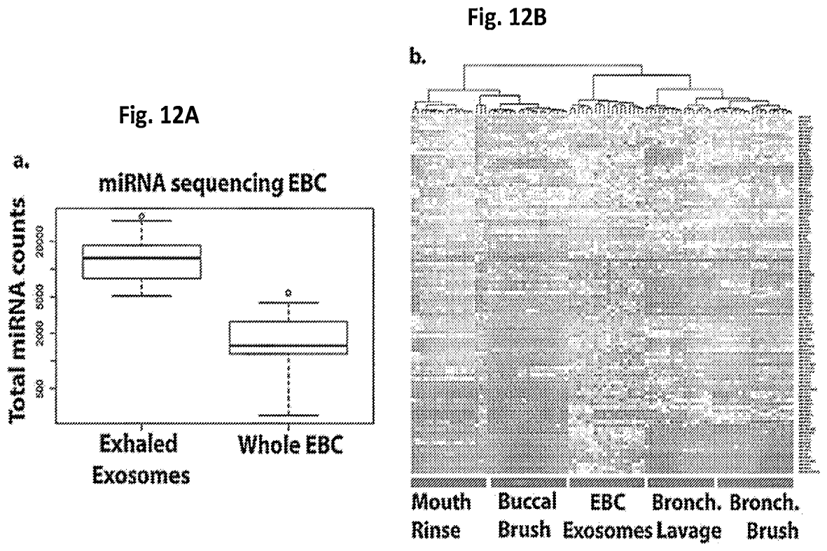

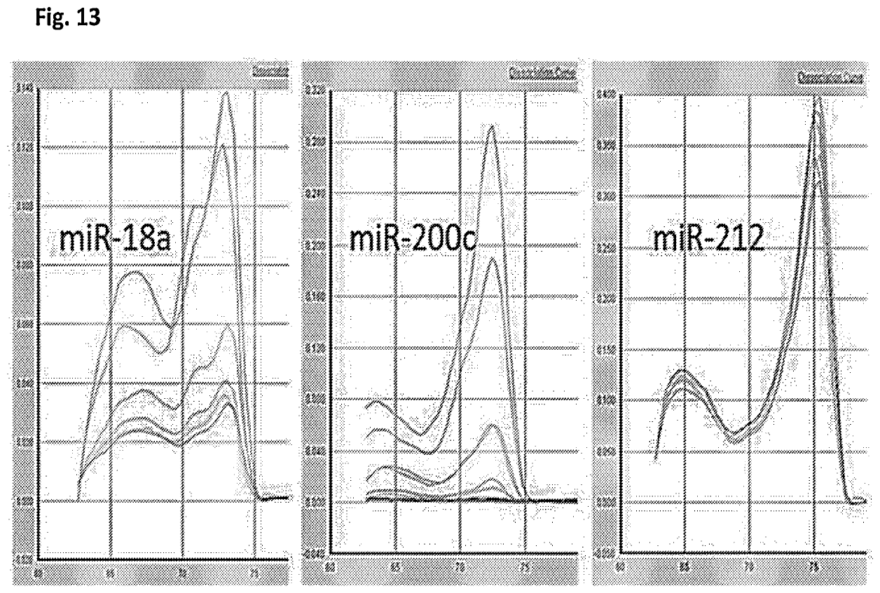

EBC is collected by a subject performing tidal (normal, quiet) breathing over a standard period of time. The exhaled breath can be diverted and condensed in a cooling chamber, using lab constructed or commercially available means. This aqueous EBC material can be analyzed as a crude unpartitioned sample, or partitioned to exosomes and supernatant. In some embodiments, a technology for exosome capture where necessary, EV-CATCHER (Mitchell et al. ((2021) J Extracell Vesicles 10: e12110; US2022/0205990; and WO2022/140662; each of which is incorporated herein by reference) (FIG. 11), may be used. Notably, the exosome-capture approach conferred 10-fold increased sensitivity (#reads per exhaled microRNA sample) as compared to non-partitioned exhaled microRNA samples (FIG. 12). Additionally, the exosome partitioned EBCmicroRNA samples clustered closely with deep lung specimens, in an unsupervised logistic regression (dendrogram) analysis. Thus, for miRNA that is particularly low in abundance, partitioning the EBC to an exosomal fraction may enhance sensitivity.

The EBC sample may be then extracted for nucleic acids, optionally followed by size separation for focus on microRNA expression evaluations. The microRNA fraction (exosomal or non-exosomal) can be analyzed by next generation sequencing, PCR, or analogous strategies described herein or known in the art.

The samples can be collected from individuals repeatedly over a longitudinal period of time (e.g., once or more on the order of days, weeks, months, annually, biannually, etc.).

Sample preparation and separation can involve any of the procedures, depending on the type of sample collected and/or analysis of biomarker measurement(s). Such procedures include, by way of example only, concentration, dilution, adjustment of pH, removal of high abundance polypeptides (e.g., albumin, gamma globulin, and transferrin, etc.), addition of preservatives and calibrants, addition of protease inhibitors, addition of RNase inhibitor, addition of denaturants, desalting of samples, concentration of sample RNA, extraction and purification of miRNA.

As described above, EBC may be partitioned into an exosomal fraction or a non-exosomal fraction (e.g., supernatant of EBC). In some embodiments, the exosomal fraction, e.g., small extracellular vesicles (EVs) comprising miRNA, can be separated based on physical properties (e.g., size, weight). In some embodiments, the EV-CATCHER technology is used to prepare the exosomal fraction.

EV-CATCHER stands for “Extracellular Vesicle Capture by AnTibody of CHoice and Enzymatic Release,” which allows for customizable selection and release of immuno-purified small-EVs. For example, antibodies targeting tetraspanins expressed on small-EVs (anti-CD63, -CD9, -CD81 antibodies) can be used to affinity-capture small EVs comprising miRNAs from a crude sample (e.g., EBC), thereby concentrating the trace amount of miRNAs present in the sample.

As described herein, in some embodiments, the level of miRNA measurement(s) in a sample from a subject is compared to a control biological sample (e.g., biological sample from a subject who does not have a cancer), a control biological sample from the subject during remission or before developing a cancer, or a control biological sample from the subject during treatment for developing a cancer. In some embodiments, a control biological sample is from a subject prior to treatment with a certain therapy. In some embodiments, wherein a subject is treated with multiple rounds of one or more therapies, a control biological sample may be from an earlier or later time point with respect to the subject sample during such treatment. For example, a subject sample after a third round of therapy may be compared with a control subject sample after the first round of therapy.

In some embodiments, the level of miRNA measurement(s) in a sample from a subject is compared to a predetermined control (standard) sample. The sample from the subject is typically from a diseased subject. The control sample can be from the same subject or from a different subject. The control sample can be from a normal, non-diseased subject. However, in some embodiments, such as for staging of disease or for evaluating the efficacy of treatment, the control sample can be from a diseased subject. The control sample can be a combination of samples from several different subjects. In some embodiments, the biomarker amount and/or activity measurement(s) from a subject is compared to a pre-determined level. This pre-determined level is typically obtained from normal samples.

As described herein, a “pre-determined” biomarker amount measurement(s) may be a biomarker amount measurement(s) used to, by way of example only, evaluate a subject that may be selected for treatment, evaluate a response to a cancer therapy, and/or evaluate a response to a an anti-cancer therapy. A pre-determined biomarker amount measurement(s) may be determined in populations of patients with or without cancer. The pre-determined biomarker amount measurement(s) can be a single number, equally applicable to every patient, or the pre-determined biomarker amount measurement(s) can vary according to specific subpopulations of patients. Age, weight, height, and other factors of a subject may affect the pre-determined biomarker amount measurement(s) of the individual. Furthermore, the pre-determined biomarker amount can be determined for each subject individually. In some embodiments, the amounts determined and/or compared in a method described herein are based on absolute measurements.

In some embodiments, the amounts determined and/or compared in a method described herein are based on relative measurements, such as ratios (e.g., biomarker level before a treatment vs. after a treatment, such biomarker measurements relative to a spiked or man-made control, such biomarker measurements relative to the expression of a housekeeping gene, and the like). For example, the relative analysis can be based on the ratio of pre-treatment biomarker measurement as compared to post-treatment biomarker measurement. Pre-treatment biomarker measurement can be made at any time prior to initiation of anti-cancer therapy. Post-treatment biomarker measurement can be made at any time after initiation of anti-cancer therapy. In some embodiments, post-treatment biomarker measurements are made 1, 2, 3, 4, 5, 6, 7, 8, 9, 10, 11, 12, 13, 14, 15, 16, 17, 18, 19, 20 weeks or more after initiation of anti-cancer therapy, and even longer toward indefinitely for continued monitoring. Treatment can comprise one or more anti-cancer therapies.

The pre-determined biomarker amount measurement(s) can be any suitable standard. For example, the pre-determined biomarker amount measurement(s) can be obtained from the same or a different human for whom a patient selection is being assessed. In some embodiments, the pre-determined biomarker amount measurement(s) can be obtained from a previous assessment of the same patient. In such a manner, the progress of the selection of the patient can be monitored over time. In addition, the control can be obtained from an assessment of another human or multiple humans, e.g., selected groups of humans, if the subject is a human. In such a manner, the extent of the selection of the human for whom selection is being assessed can be compared to suitable other humans, e.g., other humans who are in a similar situation to the human of interest, such as those suffering from similar or the same condition(s) and/or of the same ethnic group.

In some embodiments of the present disclosure the change of biomarker amount measurement(s) from the pre-determined level is about 0.1, 0.2, 0.3, 0.4, 0.5, 0.6, 0.7, 0.8, 0.9, 1.0, 1.5, 2.0, 2.5, 3.0, 3.5, 4.0, 4.5, or 5.0 fold or greater, or any range in between, inclusive. Such cutoff values apply equally when the measurement is based on relative changes, such as based on the ratio of pre-treatment biomarker measurement as compared to post-treatment biomarker measurement.

### Cancer

Cancer, tumor, or hyperproliferative disorder refer to the presence of cells possessing characteristics typical of cancer-causing cells, such as uncontrolled proliferation, immortality, metastatic potential, rapid growth and proliferation rate, and certain characteristic morphological features. Cancer cells are often in the form of a tumor, but such cells may exist alone within an animal, or may be a non-tumorigenic cancer cell, such as a leukemia cell.

In certain embodiments, cancers include lung cancer such as adenocarcinoma, squamous cell carcinoma, small cell lung cancer (SCLC), non-small cell lung cancer (NSCLC) (e.g., undifferentiated), or metastases from another organs to a lung.

The metastases from the cancer of other organs to a lung may originate from any one of the following cancer types, including, but are not limited to, B cell cancer, e.g., multiple myeloma, Waldenström's macroglobulinemia, the heavy chain diseases, such as, for example, alpha chain disease, gamma chain disease, and mu chain disease, benign monoclonal gammopathy, and immunocytic amyloidosis, melanomas, breast cancer, lung cancer, bronchus cancer, colorectal cancer, prostate cancer, pancreatic cancer, stomach cancer, ovarian cancer, urinary bladder cancer, brain or central nervous system cancer, peripheral nervous system cancer, esophageal cancer, cervical cancer, uterine or endometrial cancer, cancer of the oral cavity or pharynx, liver cancer, kidney cancer, testicular cancer, biliary tract cancer, small bowel or appendix cancer, salivary gland cancer, thyroid gland cancer, adrenal gland cancer, osteosarcoma, chondrosarcoma, cancer of hematologic tissues, and the like. Other non-limiting examples of types of cancers applicable to the methods encompassed by the present invention include human sarcomas and carcinomas, e.g., fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, colorectal cancer, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, liver cancer, choriocarcinoma, seminoma, embryonal carcinoma, Wilms' tumor, cervical cancer, bone cancer, brain tumor, testicular cancer, lung carcinoma, small cell lung carcinoma (SCLC), bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, neuroblastoma, retinoblastoma; leukemias, e.g., acute lymphocytic leukemia and acute myelocytic leukemia (myeloblastic, promyelocytic, myelomonocytic, monocytic and erythroleukemia); chronic leukemia (chronic myelocytic (granulocytic) leukemia and chronic lymphocytic leukemia); and polycythemia vera, lymphoma (Hodgkin's disease and non-Hodgkin's disease), multiple myeloma, Waldenstrom's macroglobulinemia, and heavy chain disease. In some embodiments, cancers are epithelial in nature and include but are not limited to, bladder cancer, breast cancer, cervical cancer, colon cancer, gynecologic cancers, renal cancer, laryngeal cancer, lung cancer, oral cancer, head and neck cancer, ovarian cancer, pancreatic cancer, prostate cancer, or skin cancer. In other embodiments, the cancer is breast cancer, prostate cancer, lung cancer, or colon cancer. In still other embodiments, the epithelial cancer is non-small-cell lung cancer, nonpapillary renal cell carcinoma, cervical carcinoma, ovarian carcinoma (e.g., serous ovarian carcinoma), or breast carcinoma. The epithelial cancers may be characterized in various other ways including, but not limited to, serous, endometrioid, mucinous, clear cell, Brenner, or undifferentiated.

### Cancer Therapy

The therapeutic agents of the present invention can be used alone or can be administered in combination therapy with, e.g., chemotherapeutic agents, hormones, antiangiogens, radiolabelled, compounds, or with surgery, cryotherapy, immunotherapy, cancer vaccine, immune cell engineering (e.g., CAR-T), and/or radiotherapy. The preceding treatment methods can be administered in conjunction with other forms of conventional therapy (e.g., standard-of-care treatments for cancer well-known to the skilled artisan), either consecutively with, pre- or post-conventional therapy. For example, agents of the present invention can be administered with a therapeutically effective dose of chemotherapeutic agent. In other embodiments, agents of the present invention are administered in conjunction with chemotherapy to enhance the activity and efficacy of the chemotherapeutic agent. The Physicians' Desk Reference (PDR) discloses dosages of chemotherapeutic agents that have been used in the treatment of various cancers. The dosing regimen and dosages of these aforementioned chemotherapeutic drugs that are therapeutically effective will depend on the particular cancer being treated, the extent of the disease and other factors familiar to the physician of skill in the art, and can be determined by the physician.

Immunotherapy is a targeted therapy that may comprise, for example, the use of cancer vaccines and/or sensitized antigen presenting cells. For example, an oncolytic virus is a virus that is able to infect and lyse cancer cells, while leaving normal cells unharmed, making them potentially useful in cancer therapy. Replication of oncolytic viruses both facilitates tumor cell destruction and also produces dose amplification at the tumor site. They may also act as vectors for anticancer genes, allowing them to be specifically delivered to the tumor site. The immunotherapy can involve passive immunity for short-term protection of a host, achieved by the administration of pre-formed antibody directed against a cancer antigen or disease antigen (e.g., administration of a monoclonal antibody, optionally linked to a chemotherapeutic agent or toxin, to a tumor antigen). For example, anti-VEGF is known to be effective in treating renal cell carcinoma. Immunotherapy can also focus on using the cytotoxic lymphocyte-recognized epitopes of cancer cell lines. Alternatively, antisense polynucleotides, ribozymes, RNA interference molecules, triple helix polynucleotides and the like, can be used to selectively modulate biomolecules that are linked to the initiation, progression, and/or pathology of a tumor or cancer.

Immunotherapy also encompasses immune checkpoint modulators. Immune checkpoints are a group of molecules on the cell surface of CD4+ and/or CD8+ T cells that fine-tune immune responses by down-modulating or inhibiting an anti-tumor immune response. Immune checkpoint proteins are well-known in the art and include, without limitation, CTLA-4, PD-1, VISTA, B7-H2, B7-H3, PD-L1, B7-H4, B7-H6, 2B4, ICOS, HVEM, PD-L2, CD160, gp49B, PIR-B, KIR family receptors, TIM-1, TIM-3, TIM-4, LAG-3, BTLA, SIRPalpha (CD47), CD48, 2B4 (CD244), B7.1, B7.2, ILT-2, ILT-4, TIGIT, HHLA2, TMIDG2, KIR3DL3, and A2aR (see, for example, WO 2012/177624). Inhibition of one or more immune checkpoint inhibitors can block or otherwise neutralize inhibitory signaling to thereby upregulate an immune response in order to more efficaciously treat cancer. In some embodiments, the cancer vaccine is administered in combination with one or more inhibitors of immune checkpoints (immune checkpoint inhibition therapy), such as PD1, PD-L1, and/or CD47 inhibitors.

Adoptive cell-based immunotherapies can be combined with the therapies of the present invention. Well-known adoptive cell-based immunotherapeutic modalities, including, without limitation, irradiated autologous or allogeneic tumor cells, tumor lysates or apoptotic tumor cells, antigen-presenting cell-based immunotherapy, dendritic cell-based immunotherapy, adoptive T cell transfer, adoptive CAR T cell therapy, autologous immune enhancement therapy (AIET), cancer vaccines, and/or antigen presenting cells. Such cell-based immunotherapies can be further modified to express one or more gene products to further modulate immune responses, such as expressing cytokines like GM-CSF, and/or to express tumor-associated antigen (TAA) antigens, such as Mage-1, gp-100, and the like.

The term “chimeric antigen receptor” or “CAR” refers to engineered T cell receptors (TCR) having a desired antigen specificity. T lymphocytes recognize specific antigens through interaction of the T cell receptor (TCR) with short peptides presented by major histocompatibility complex (MHC) class I or II molecules. For initial activation and clonal expansion, naive T cells are dependent on professional antigen-presenting cells (APCs) that provide additional co-stimulatory signals. TCR activation in the absence of co-stimulation can result in unresponsiveness and clonal anergy. To bypass immunization, different approaches for the derivation of cytotoxic effector cells with grafted recognition specificity have been developed. CARs have been constructed that consist of binding domains derived from natural ligands or antibodies specific for cell-surface components of the TCR-associated CD3 complex. Upon antigen binding, such chimeric antigen receptors link to endogenous signaling pathways in the effector cell and generate activating signals similar to those initiated by the TCR complex. Since the first reports on chimeric antigen receptors, this concept has steadily been refined and the molecular design of chimeric receptors has been optimized and routinely use any number of well-known binding domains, such as scFV and another protein binding fragments described herein.

In other embodiments, immunotherapy comprises non-cell-based immunotherapies. In some embodiments, compositions comprising antigens with or without vaccine-enhancing adjuvants are used. Such compositions exist in many well-known forms, such as peptide compositions, oncolytic viruses, recombinant antigen comprising fusion proteins, and the like. In some embodiments, immunomodulatory cytokines, such as interferons, G-CSF, imiquimod, TNFalpha, and the like, as well as modulators thereof (e.g., blocking antibodies or more potent or longer lasting forms) are used. In some embodiments, immunomodulatory interleukins, such as IL-2, IL-6, IL-7, IL-12, IL-17, IL-23, and the like, as well as modulators thereof (e.g., blocking antibodies or more potent or longer lasting forms) are used. In some embodiments, immunomodulatory chemokines, such as CCL3, CCL26, and CXCL7, and the like, as well as modulators thereof (e.g., blocking antibodies or more potent or longer lasting forms) are used. In some embodiments, immunomodulatory molecules targeting immunosuppression, such as STAT3 signaling modulators, NFkappaB signaling modulators, and immune checkpoint modulators, are used.

In still other embodiments, immunomodulatory drugs, such as immunocytostatic drugs, glucocorticoids, cytostatics, immunophilins and modulators thereof (e.g., rapamycin, a calcineurin inhibitor, tacrolimus, ciclosporin (cyclosporin), pimecrolimus, abetimus, gusperimus, ridaforolimus, everolimus, temsirolimus, zotarolimus, etc.), hydrocortisone (cortisol), cortisone acetate, prednisone, prednisolone, methylprednisolone, dexamethasone, betamethasone, triamcinolone, beclometasone, fludrocortisone acetate, deoxycorticosterone acetate (doca) aldosterone, a non-glucocorticoid steroid, a pyrimidine synthesis inhibitor, leflunomide, teriflunomide, a folic acid analog, methotrexate, anti-thymocyte globulin, anti-lymphocyte globulin, thalidomide, lenalidomide, pentoxifylline, bupropion, curcumin, catechin, an opioid, an IMPDH inhibitor, mycophenolic acid, myriocin, fingolimod, an NF-xB inhibitor, raloxifene, drotrecogin alfa, denosumab, an NF-xB signaling cascade inhibitor, disulfiram, olmesartan, dithiocarbamate, a proteasome inhibitor, bortezomib, MG132, Prol, NPI-0052, curcumin, genistein, resveratrol, parthenolide, thalidomide, lenalidomide, flavopiridol, non-steroidal anti-inflammatory drugs (NSAIDs), arsenic trioxide, dehydroxymethylepoxyquinomycin (DHMEQ), I3C (indole-3-carbinol)/DIM (di-indolmethane) (13C/DIM), Bay 11-7082, luteolin, cell permeable peptide SN-50, IKBa.-super repressor overexpression, NFKB decoy oligodeoxynucleotide (ODN), or a derivative or analog of any thereo, are used. In yet other embodiments, immunomodulatory antibodies or protein are used. For example, antibodies that bind to CD40, Toll-like receptor (TLR), OX40, GITR, CD27, or to 4-1BB, T-cell bispecific antibodies, an anti-IL-2 receptor antibody, an anti-CD3 antibody, OKT3 (muromonab), otelixizumab, teplizumab, visilizumab, an anti-CD4 antibody, clenoliximab, keliximab, zanolimumab, an anti-CD11 a antibody, efalizumab, an anti-CD18 antibody, erlizumab, rovelizumab, an anti-CD20 antibody, afutuzumab, ocrelizumab, ofatumumab, pascolizumab, rituximab, an anti-CD23 antibody, lumiliximab, an anti-CD40 antibody, teneliximab, toralizumab, an anti-CD40L antibody, ruplizumab, an anti-CD62L antibody, aselizumab, an anti-CD80 antibody, galiximab, an anti-CD147 antibody, gavilimomab, a B-Lymphocyte stimulator (BLyS) inhibiting antibody, belimumab, an CTLA4-Ig fusion protein, abatacept, belatacept, an anti-CTLA4 antibody, ipilimumab, tremelimumab, an anti-eotaxin 1 antibody, bertilimumab, an anti-a4-integrin antibody, natalizumab, an anti-IL-6R antibody, tocilizumab, an anti-LFA-1 antibody, odulimomab, an anti-CD25 antibody, basiliximab, daclizumab, inolimomab, an anti-CD5 antibody, zolimomab, an anti-CD2 antibody, siplizumab, nerelimomab, faralimomab, atlizumab, atorolimumab, cedelizumab, dorlimomab aritox, dorlixizumab, fontolizumab, gantenerumab, gomiliximab, lebrilizumab, maslimomab, morolimumab, pexelizumab, reslizumab, rovelizumab, talizumab, telimomab aritox, vapaliximab, vepalimomab, aflibercept, alefacept, rilonacept, an IL-1 receptor antagonist, anakinra, an anti-IL-5 antibody, mepolizumab, an IgE inhibitor, omalizumab, talizumab, an IL 12 inhibitor, an IL23 inhibitor, ustekinumab, and the like.

Nutritional supplements that enhance immune responses, such as vitamin A, vitamin E, vitamin C, and the like, are well-known in the art (see, for example, U.S. Pat. Nos. 4,981,844 and 5,230,902 and PCT Publ. No. WO 2004/004483) can be used in the methods described herein.

Similarly, various agents or a combination thereof can be used to treat a cancer. For example, chemotherapy, radiation, epigenetic modifiers (e.g., histone deacetylase (HDAC) modifiers, methylation modifiers, phosphorylation modifiers, and the like), targeted therapy, and the like are well-known in the art.

In some embodiments, chemotherapy is used. Chemotherapy includes the administration of a chemotherapeutic agent. Such a chemotherapeutic agent may be, but is not limited to, those selected from among the following groups of compounds: platinum compounds, cytotoxic antibiotics, antimetabolites, anti-mitotic agents, alkylating agents, arsenic compounds, DNA topoisomerase inhibitors, taxanes, nucleoside analogues, plant alkaloids, and toxins; and synthetic derivatives thereof. Exemplary compounds include, but are not limited to, alkylating agents: cisplatin, treosulfan, and trofosfamide; plant alkaloids: vinblastine, paclitaxel, docetaxol; DNA topoisomerase inhibitors: teniposide, crisnatol, and mitomycin; anti-folates: methotrexate, mycophenolic acid, and hydroxyurea; pyrimidine analogs: 5-fluorouracil, doxifluridine, and cytosine arabinoside; purine analogs: mercaptopurine and thioguanine; DNA antimetabolites: 2′-deoxy-5-fluorouridine, aphidicolin glycinate, and pyrazoloimidazole; and antimitotic agents: halichondrin, colchicine, and rhizoxin. Compositions comprising one or more chemotherapeutic agents (e.g., FLAG, CHOP) may also be used. FLAG comprises fludarabine, cytosine arabinoside (Ara-C) and G-CSF. CHOP comprises cyclophosphamide, vincristine, doxorubicin, and prednisone. In another embodiments, PARP (e.g., PARP-1 and/or PARP-2) inhibitors are used and such inhibitors are well-known in the art (e.g., Olaparib, ABT-888, BSI-201, BGP-15 (N-Gene Research Laboratories, Inc.); INO-1001 (Inotek Pharmaceuticals Inc.); PJ34 (Soriano et al., 2001; Pacher et al., 2002b); 3-aminobenzamide (Trevigen); 4-amino-1,8-naphthalimide; (Trevigen); 6 (5H)-phenanthridinone (Trevigen); benzamide (U.S. Pat. Re. 36,397); and NU1025 (Bowman et al.). The mechanism of action is generally related to the ability of PARP inhibitors to bind PARP and decrease its activity. PARP catalyzes the conversion of .beta.-nicotinamide adenine dinucleotide (NAD+) into nicotinamide and poly-ADP-ribose (PAR). Both poly (ADP-ribose) and PARP have been linked to regulation of transcription, cell proliferation, genomic stability, and carcinogenesis (Bouchard V. J. et.al. Experimental Hematology, Volume 31, Number 6, June 2003, pp. 446-454 (9); Herceg Z.; Wang Z.-Q. Mutation Research/Fundamental and Molecular Mechanisms of Mutagenesis, Volume 477, Number 1, 2 Jun. 2001, pp. 97-110 (14)). Poly (ADP-ribose) polymerase 1 (PARP1) is a key molecule in the repair of DNA single-strand breaks (SSBs) (de Murcia J. et al. 1997. Proc Natl Acad Sci USA 94:7303-7307; Schreiber V, Dantzer F, Ame J C, de Murcia G (2006) Nat Rev Mol Cell Biol 7:517-528; Wang Z Q, et al. (1997) Genes Dev 11:2347-2358). Knockout of SSB repair by inhibition of PARP1 function induces DNA double-strand breaks (DSBs) that can trigger synthetic lethality in cancer cells with defective homology-directed DSB repair (Bryant H E, et al. (2005) Nature 434:913-917; Farmer H, et al. (2005) Nature 434:917-921). The foregoing examples of chemotherapeutic agents are illustrative, and are not intended to be limiting.

In other embodiments, radiation therapy is used. The radiation used in radiation therapy can be ionizing radiation. Radiation therapy can also be gamma rays, X-rays, or proton beams. Examples of radiation therapy include, but are not limited to, external-beam radiation therapy, interstitial implantation of radioisotopes (I-125, palladium, iridium), radioisotopes such as strontium-89, thoracic radiation therapy, intraperitoneal P-32 radiation therapy, and/or total abdominal and pelvic radiation therapy. For a general overview of radiation therapy, see Hellman, Chapter 16: Principles of Cancer Management: Radiation Therapy, 6th edition, 2001, DeVita et al., eds., J. B. Lippencott Company, Philadelphia. The radiation therapy can be administered as external beam radiation or teletherapy wherein the radiation is directed from a remote source. The radiation treatment can also be administered as internal therapy or brachytherapy wherein a radioactive source is placed inside the body close to cancer cells or a tumor mass. Also encompassed is the use of photodynamic therapy comprising the administration of photosensitizers, such as hematoporphyrin and its derivatives, Vertoporfin (BPD-MA), phthalocyanine, photosensitizer Pc4, demethoxy-hypocrellin A; and 2BA-2-DMHA.

In other embodiments, hormone therapy is used. Hormonal therapeutic treatments can comprise, for example, hormonal agonists, hormonal antagonists (e.g., flutamide, bicalutamide, tamoxifen, raloxifene, leuprolide acetate (LUPRON), LH-RH antagonists), inhibitors of hormone biosynthesis and processing, and steroids (e.g., dexamethasone, retinoids, deltoids, betamethasone, cortisol, cortisone, prednisone, dehydrotestosterone, glucocorticoids, mineralocorticoids, estrogen, testosterone, progestins), vitamin A derivatives (e.g., all-trans retinoic acid (ATRA)); vitamin D3 analogs; antigestagens (e.g., mifepristone, onapristone), or antiandrogens (e.g., cyproterone acetate).

In other embodiments, photodynamic therapy (also called PDT, photoradiation therapy, phototherapy, or photochemotherapy) is used for the treatment of some types of cancer. It is based on the discovery that certain chemicals known as photosensitizing agents can kill one-celled organisms when the organisms are exposed to a particular type of light.

In yet other embodiments, laser therapy is used to harness high-intensity light to destroy cancer cells. This technique is often used to relieve symptoms of cancer such as bleeding or obstruction, especially when the cancer cannot be cured by other treatments. It may also be used to treat cancer by shrinking or destroying tumors.

Asthma and Therapies that Treats Asthma

Asthma is a condition in which the airways narrow and swell and may produce extra mucus. This can make breathing difficult and trigger coughing, a whistling sound (wheezing) when you breathe out and shortness of breath.

Preventive, long-term control medications reduce the swelling (inflammation) in the airways that leads to symptoms. Quick-relief inhalers (bronchodilators) quickly open swollen airways that are limiting breathing. In some cases, allergy medications are necessary.

Long-term asthma control medications, generally taken daily, are the cornerstone of asthma treatment. These medications keep asthma under control on a day-to-day basis and make an asthma attack less likely. Types of long-term control medications include:
Inhaled corticosteroids. These medications include fluticasone propionate (Flovent HFA, Flovent Diskus, Xhance), budesonide (Pulmicort Flexhaler, Pulmicort Respules, Rhinocort), ciclesonide (Alvesco), beclomethasone (Qvar Redihaler), mometasone (Asmanex HFA, Asmanex Twisthaler) and fluticasone furoate (Arnuity Ellipta).

A subject may need to use these medications for several days to weeks before they reach their maximum benefit. Unlike oral corticosteroids, inhaled corticosteroids have a relatively low risk of serious side effects.
Leukotriene modifiers. These oral medications—including montelukast (Singulair), zafirlukast (Accolate) and zileuton (Zyflo)—help relieve asthma symptoms.
Combination inhalers. These medications—such as fluticasone-salmeterol (Advair HFA, Airduo Digihaler, others), budesonide-formoterol (Symbicort), formoterol-mometasone (Dulera) and fluticasone furoate-vilanterol (Breo Ellipta)—contain a long-acting beta agonist along with a corticosteroid.
Theophylline. Theophylline (Theo-24, Elixophyllin, Theochron) is a daily pill that helps keep the airways open by relaxing the muscles around the airways. It's not used as often as other asthma medications and requires regular blood tests.

Quick-relief (rescue) medications are used as needed for rapid, short-term symptom relief during an asthma attack. They may also be used before exercise if the doctor recommends it. Types of quick-relief medications include:
Beta agonists. These inhaled, quick-relief bronchodilators act within minutes to rapidly ease symptoms during an asthma attack. They include albuterol (ProAir HFA, Ventolin HFA, others) and levalbuterol (Xopenex, Xopenex HFA).

Beta agonists can be taken using a portable, hand-held inhaler or a nebulizer, a machine that converts asthma medications to a fine mist. They are inhaled through a face mask or mouthpiece.
Anticholinergic agents. Like other bronchodilators, ipratropium (Atrovent HFA) and tiotropium (Spiriva, Spiriva Respimat) act quickly to immediately relax your airways, making it easier to breathe. They are mostly used for emphysema and chronic bronchitis, but can be used to treat asthma.
Oral and intravenous corticosteroids. These medications-which include prednisone (Prednisone Intensol, Rayos) and methylprednisolone (Medrol, Depo-Medrol, Solu-Medrol)-relieve airway inflammation caused by severe asthma. They can cause serious side effects when used long term, so these drugs are used only on a short-term basis to treat severe asthma symptoms.

Allergy medications may help if the asthma is triggered or worsened by allergies. These include:
Allergy shots (immunotherapy). Over time, allergy shots gradually reduce the immune system reaction to specific allergens. A subject generally receives shots once a week for a few months, then once a month for a period of three to five years.
Biologies. These medications—which include omalizumab (Xolair), mepolizumab (Nucala), dupilumab (Dupixent), reslizumab (Cinqair) and benralizumab (Fasenra) are specifically for people who have severe asthma.

Bronchial thermoplasty is used for severe asthma that doesn't improve with inhaled corticosteroids or other long-term asthma medications. During bronchial thermoplasty, a doctor heats the insides of the airways in the lungs with an electrode. The heat reduces the smooth muscle inside the airways. This limits the ability of the airways to tighten, making breathing easier and possibly reducing asthma attacks. The therapy is generally done over three outpatient visits.

Chronic Obstructive Pulmonary Disease (COPD) and Therapies that Treat COPD

Chronic obstructive pulmonary disease (COPD) is a chronic inflammatory lung disease that causes obstructed airflow from the lungs. Symptoms include breathing difficulty, cough, mucus (sputum) production and wheezing. It is typically caused by long-term exposure to irritating gases or particulate matter, most often from cigarette smoke. People with COPD are at increased risk of developing heart disease, lung cancer and a variety of other conditions.

Emphysema and chronic bronchitis are the two most common conditions that contribute to COPD. These two conditions usually occur together and can vary in severity among individuals with COPD.

Chronic bronchitis is inflammation of the lining of the bronchial tubes, which carry air to and from the air sacs (alveoli) of the lungs. It is characterized by daily cough and mucus (sputum) production.

Emphysema is a condition in which the alveoli at the end of the smallest air passages (bronchioles) of the lungs are destroyed as a result of damaging exposure to cigarette smoke and other irritating gases and particulate matter.

Although COPD is a progressive disease that gets worse over time, COPD is treatable. With proper management, most people with COPD can achieve good symptom control and quality of life, as well as reduced risk of other associated conditions.

Several kinds of medications are used to treat the symptoms and complications of COPD.

### I. Bronchodilators

Bronchodilators are medications that usually come in inhalers-they relax the muscles around your airways. This can help relieve coughing and shortness of breath and make breathing easier. Depending on the severity of the disease, a subject may need a short-acting bronchodilator before activities, a long-acting bronchodilator that the subject uses every day or both.

Examples of short-acting bronchodilators include:
Albuterol (ProAir HFA, Ventolin HFA, others)
Ipratropium (Atrovent HFA)
Levalbuterol (Xopenex)

Examples of long-acting bronchodilators include:
Aclidinium (Tudorza Pressair)
Arformoterol (Brovana)
Formoterol (Perforomist)
Indacaterol (Arcapta Neoinhaler)
Tiotropium (Spiriva)
Salmeterol (Serevent)
Umeclidinium (Incruse Ellipta)

### II. Inhaled Steroids

Inhaled corticosteroid medications can reduce airway inflammation and help prevent exacerbations. Side effects may include bruising, oral infections and hoarseness.

These medications are useful for people with frequent exacerbations of COPD. Examples of inhaled steroids include:
Fluticasone (Flovent HFA)
Budesonide (Pulmicort Flexhaler)

### III. Combination Inhalers

Some medications combine bronchodilators and inhaled steroids. Examples of these combination inhalers include:
Fluticasone and vilanterol (Breo Ellipta)
Fluticasone, umeclidinium and vilanterol (Trelegy Ellipta)
Formoterol and budesonide (Symbicort)
Salmeterol and fluticasone (Advair HFA, AirDuo Digihaler, others)

Combination inhalers that include more than one type of bronchodilator also are available. Examples of these include:
Aclidinium and formoterol (Duaklir Pressair)
Albuterol and ipratropium (Combivent Respimat)
Formoterol and glycopyrrolate (Bevespi Aerosphere)
Glycopyrrolate and indacaterol (Utibron)
Olodaterol and tiotropium (Stiolto Respimat)
Umeclidinium and vilanterol (Anoro Ellipta)

### IV. Oral Steroids

For people who experience periods when their COPD becomes more severe, called moderate or severe acute exacerbation, short courses (for example, five days) of oral corticosteroids may prevent further worsening of COPD. However, long-term use of these medications can have serious side effects, such as weight gain, diabetes, osteoporosis, cataracts and an increased risk of infection.

### V. Phosphodiesterase-4 Inhibitors

A medication approved for people with severe COPD and symptoms of chronic bronchitis is roflumilast (Daliresp), a phosphodiesterase-4 inhibitor. This drug decreases airway inflammation and relaxes the airways. Common side effects include diarrhea and weight loss.

### VI. Theophylline

When other treatment has been ineffective or if cost is a factor, theophylline (Elixophyllin, Theo-24, Theochron), a less expensive medication, may help improve breathing and prevent episodes of worsening COPD. Side effects are dose related and may include nausea, headache, fast heartbeat and tremor, so tests are used to monitor blood levels of the medication.

### VII. Antibiotics

Respiratory infections, such as acute bronchitis, pneumonia and influenza, can aggravate COPD symptoms. Antibiotics help treat episodes of worsening COPD, but they aren't generally recommended for prevention. Some studies show that certain antibiotics, such as azithromycin (Zithromax), prevent episodes of worsening COPD, but side effects and antibiotic resistance may limit their use.

### VIII. Lung Therapies

Doctors often use these additional therapies for people with moderate or severe COPD:
Oxygen therapy. If there isn't enough oxygen in your blood, you may need supplemental oxygen. There are several devices that deliver oxygen to your lungs, including lightweight, portable units that you can take with you to run errands and get around town.

Some people with COPD use oxygen only during activities or while sleeping. Others use oxygen all the time. Oxygen therapy can improve quality of life and is the only COPD therapy proved to extend life.
Pulmonary rehabilitation program. These programs generally combine education, exercise training, nutrition advice and counseling. Pulmonary rehabilitation after episodes of worsening COPD may reduce readmission to the hospital, increase a subject's ability to participate in everyday activities and improve quality of life.

### IX. In-Home Noninvasive Ventilation Therapy

Evidence supports in-hospital use of breathing devices such as bilevel positive airway pressure (BiPAP), but some research now supports the benefit of its use at home. A noninvasive ventilation therapy machine with a mask helps to improve breathing and decrease retention of carbon dioxide (hypercapnia) that may lead to acute respiratory failure and hospitalization.

### X. Managing Exacerbations

Even with ongoing treatment, a subject may experience times when symptoms become worse for days or weeks. This is called an acute exacerbation, and it may lead to lung failure if the subject does not receive prompt treatment.

Exacerbations may be caused by a respiratory infection, air pollution or other triggers of inflammation. Whatever the cause, it is important to seek prompt medical help if a sustained increase in coughing or a change in the mucus occurs. When exacerbations occur, a subject may need additional medications (such as antibiotics, steroids or both), supplemental oxygen or treatment in the hospital.

### XI. Surgery

Surgery is an option for some people with some forms of severe emphysema who are not helped sufficiently by medications alone. Surgical options include:
Lung volume reduction surgery. In this surgery, a surgeon removes small wedges of damaged lung tissue from the upper lungs. This creates extra space in your chest cavity so that the remaining healthier lung tissue can expand and the diaphragm can work more efficiently. In some people, this surgery can improve quality of life and prolong survival.

Endoscopic lung volume reduction—a minimally invasive procedure—has recently been approved by the U.S. Food and Drug Administration to treat people with COPD. A tiny one-way endobronchial valve is placed in the lung, allowing the most damaged lobe to shrink so that the healthier part of the lung has more space to expand and function.
Lung transplant. Lung transplantation may be an option for certain people who meet specific criteria. Transplantation can improve one's ability to breathe and to be active.
Bullectomy. Large air spaces (bullae) form in the lungs when the walls of the air sacs (alveoli) are destroyed. These bullae can become very large and cause breathing problems. In a bullectomy, doctors remove bullae from the lungs to help improve air flow.

### Monitoring of Effects During Clinical Trials

Monitoring the influence of agents (e.g., compounds, drugs or small molecules) on the level of miRNA can be applied not only in basic drug screening, but also in clinical trials. For example, the effectiveness of an agent determined by a screening assay as described herein to decrease the level of miRNA can be monitored in clinical trials of subjects, detectable by the methods of the present disclosure. In such clinical trials, the level of miRNA and/or symptoms or other markers of the cancer, can be used as a “read out” or marker of the phenotype of a particular cell, tissue, or system.

In preferred embodiments, the present disclosure provides a method for monitoring the effectiveness of treatment of a subject with an agent (e.g., an agonist, antagonist, peptidomimetic, polypeptide, peptide, nucleic acid, small molecule, immunotherapy, immune checkpoint inhibition therapy, or other drug candidate) including the steps of (i) obtaining a pre-administration sample (e.g., EBC or a derivative thereof) from a subject prior to administration of the agent; (ii) detecting the level of miRNA in the preadministration sample; (iii) obtaining one or more post-administration samples from the subject; (iv) detecting the level of the miRNA in the post-administration samples; (v) comparing the level of the miRNA in the pre-administration sample with the miRNA in the post administration sample or samples; and (vi) altering the administration of the agent to the subject accordingly. For example, increased administration of the agent may be desirable to decrease the level of miRNA to lower levels than detected, i.e., to increase the effectiveness of the agent. According to such embodiments, the miRNA level may be used as an indicator of the effectiveness of an agent, even in the absence of an observable phenotypic response. Similarly, miRNA can also be used to select patients who will receive a cancer therapy (e.g., immunotherapy, immune checkpoint inhibition therapy).

### Clinical Efficacy/Response to a Therapy

Clinical efficacy can be measured by any method known in the art. For example, the response to a therapy relates to any response of the cancer, e.g., a tumor, to the therapy, preferably to a change in tumor mass and/or volume after initiation of neoadjuvant or adjuvant chemotherapy. Tumor response may be assessed in a neoadjuvant or adjuvant situation where the size of a tumor after systemic intervention can be compared to the initial size and dimensions as measured by CT, PET, mammogram, ultrasound or palpation and the cellularity of a tumor can be estimated histologically and compared to the cellularity of a tumor biopsy taken before initiation of treatment. Response may also be assessed by caliper measurement or pathological examination of the tumor after biopsy or surgical resection. Response may be recorded in a quantitative fashion like percentage change in tumor volume or cellularity or using a semi-quantitative scoring system such as residual cancer burden (Symmans et al., J. Clin. Oncol. (2007) 25:4414-4422) or Miller-Payne score (Ogston et al., (2003) Breast (Edinburgh, Scotland) 12:320-327) in a qualitative fashion like “pathological complete response” (pCR), “clinical complete remission” (cCR), “clinical partial remission” (cPR), “clinical stable disease” (cSD), “clinical progressive disease” (cPD) or other qualitative criteria. Assessment of tumor response may be performed early after the onset of neoadjuvant or adjuvant therapy, e.g., after a few hours, days, weeks or preferably after a few months. A typical endpoint for response assessment is upon termination of neoadjuvant chemotherapy or upon surgical removal of residual tumor cells and/or the tumor bed.

In some embodiments, clinical efficacy of the therapeutic treatments described herein may be determined by measuring the clinical benefit rate (CBR). The clinical benefit rate is measured by determining the sum of the percentage of patients who are in complete remission (CR), the number of patients who are in partial remission (PR) and the number of patients having stable disease (SD) at a time point at least 6 months out from the end of therapy. The shorthand for this formula is CBR=CR+PR+SD over 6 months. In some embodiments, the CBR for a particular anti-immune checkpoint therapeutic regimen is at least 25%, 30%, 35%, 40%, 45%, 50%, 55%, 60%, 65%, 70%, 75%, 80%, 85%, or more.

Additional criteria for evaluating the response to a cancer therapy are related to “survival,” which includes all of the following: survival until mortality, also known as overall survival (wherein said mortality may be either irrespective of cause or tumor related); “recurrence-free survival” (wherein the term recurrence shall include both localized and distant recurrence); metastasis free survival; disease free survival (wherein the term disease shall include cancer and diseases associated therewith). The length of said survival may be calculated by reference to a defined start point (e.g., time of diagnosis or start of treatment) and end point (e.g., death, recurrence or metastasis). In addition, criteria for efficacy of treatment can be expanded to include probability of survival, probability of metastasis within a given time period, and probability of tumor recurrence.

For example, in order to determine appropriate threshold values, a particular anti-cancer therapeutic regimen can be administered to a population of subjects and the outcome can be correlated to biomarker measurements that were determined prior to administration of any cancer therapy. The outcome measurement may be pathologic response to therapy given in the neoadjuvant setting. Alternatively, outcome measures, such as overall survival and disease-free survival can be monitored over a period of time for subjects following the cancer therapy for whom biomarker measurement values are known. In certain embodiments, the same doses of anti-cancer agents are administered to each subject. In related embodiments, the doses administered are standard doses known in the art for anti-cancer agents. The period of time for which subjects are monitored can vary. For example, subjects may be monitored for at least 2, 4, 6, 8, 10, 12, 14, 16, 18, 20, 25, 30, 35, 40, 45, 50, 55, or 60 months. Biomarker measurement threshold values that correlate to outcome of a cancer therapy can be determined using methods such as those described in the Examples section.

### Exemplary Embodiments

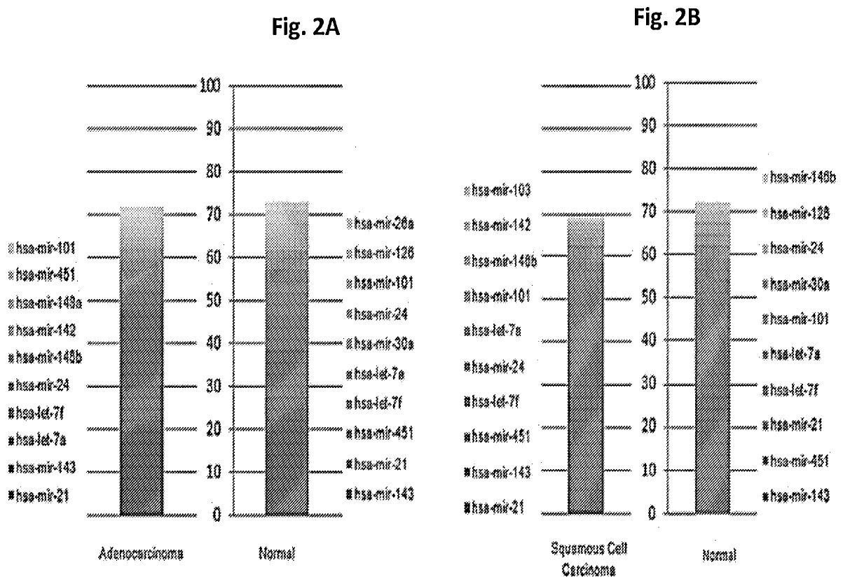

1. A method of detecting an exhaled microRNA (miRNA) of a subject, the method comprising:
(a) collecting exhaled breath condensate (EBC); and
(b) detecting the presence and/or level of the exhaled miRNA,
optionally wherein the exhaled miRNA is selected from the miRNA listed in Table 2, Table 5A, Table 5B, and FIG. 2, or any combination of two or more thereof.

2. The method of 1, wherein the exhaled miRNA comprises hsa-mir-101, hsa-mir-451, hsa-mir-148a, hsa-mir-142, hsa-mir-146b, hsa-mir-24, hsa-let-7f, hsa-let-7a, hsa-mir-143, hsa-mir-21, hsa-mir-708, hsa-mir-1259, hsa-mir-494, hsa-mir-200a, hsa-miR-135b, miRNA 33b, miRNA 212, or any combination of two or more thereof.

3. The method of 1 or 2, wherein the exhaled miRNA comprises miRNA 21, miRNA 33b, miRNA 212, or any combination of two or more thereof.

4 The method of any one of 1-3, wherein the EBC is collected by condensing the exhaled breath in a cooling chamber.

5. The method of any one of 1-4, wherein the total EBC is used for detecting the presence and/or level of the exhaled miRNA.

6. The method of any one of 1-5, wherein the EBC is partitioned to isolate an exosomal fraction (e.g., small EVs) prior to detecting the presence and/or level of the exhaled miRNA.

7. The method of 6, wherein the EBC is partitioned into an exosomal fraction using EV-CATCHER.

8. The method of any one of 1-7, wherein the exhaled miRNA detected comprises an exosomal miRNA and/or a non-exosomal miRNA.

9. The method of any one of 1-8, wherein the presence and/or level of the exhaled miRNA is detected by a method comprising: RNA-seq, next generation sequencing, sequencing, mass spectrometry (e.g., RNA sequencing by LC-MS, DNA sequencing by LC-MS), microarray, Northern blotting, reverse transcription, Southern blotting, RT-PCR, miRNA PCR, PCR (e.g., URT-PCR), realtime PCR (e.g., TaqMan®), testing for a differential melting temperature of a complementary DNA (cDNA) duplex of miRNA, any variation thereof, or any combination of two or more thereof.

10. The method of any one of 1-9, wherein the method further comprises poly-A-tailing the miRNA before detection.

11. The method of any one of 1-10, wherein the method further comprises reverse transcribing the miRNA into a cDNA, before detection.

12. The method of any one of 1-11, wherein the method comprises miRNA that is poly-A-tailed and PCR-amplified using a primer specific to the poly-A (e.g., those comprising an oligo-dT sequence) and a miRNA-specific primer, before detection.

13. The method of any one of 1-12, wherein the level of miRNA is normalized to a housekeeping miRNA present in the EBC.

14. The method of 13, wherein the housekeeping miRNA is hsa-miR-16, hsa-miR-26b, hsa-miR-92, hsa-miR-423, hsa-miR-374, or miR-423-3p, miR-21A, miR let-7a, mi R let-7f.

15. A method of diagnosing a lung disease, or risk for a lung disease, in a subject, the method comprising:
(a) detecting the presence and/or level of the exhaled miRNA according to the method of any one of 1-14; and
(b) comparing said presence and/or level of the exhaled miRNA to a control, wherein the presence and/or significantly higher level of the exhaled miRNA as compared to the control indicates that the subject has a lung disease.

16. A method of recurrence of a lung disease in a subject, the method comprising:
(a) obtaining a subject sample (e.g., EBC or any derivative thereof) from the subject who has received a therapy for the lung disease;
(b) detecting the presence and/or level of the exhaled miRNA according to the method of any one of 1-14; and
(c) comparing said presence and/or level of the exhaled miRNA to a control, wherein the presence and/or significantly higher level of the exhaled miRNA as compared to the control indicates a recurrence a lung disease in the subject.

17. A method of monitoring the progression of a lung disease in a subject, the method comprising:
(a) detecting in a subject sample (e.g., EBC or any derivative thereof) at a first point in time the presence and/or level of the exhaled miRNA according to the method of any one of 1-14;
(b) repeating step (a) at a subsequent point in time; and
(c) comparing the presence and/or level of the exhaled miRNA detected in steps (a) and (b) to monitor the progression of the lung disease in the subject.

18. The method of 17, wherein between the first point in time and the subsequent point in time, the subject has received a therapy that treats the disease (e.g., cancer therapy).

19. A method of assessing the efficacy of a therapy that treats a lung disease in a subject, the method comprising:
(a) determining the presence and/or level of the exhaled miRNA according to the method of any one of 1-14;
(b) repeating step (a) during at least one subsequent point in time after administration of the therapy; and
(c) comparing the presence and/or level of the exhaled miRNA detected in steps (a) and (b),
wherein a significantly lower level of the exhaled miRNA in the at least one subsequent sample, relative to the first sample, is an indication that the therapy is efficacious to the lung disease in the subject.

20. The method of any one of 17-19, wherein the first and/or at least one subsequent sample is a portion of a single sample or pooled samples obtained from the subject.

21. The method of any one of 15-18, wherein said significantly higher level of the exhaled miRNA comprises an at least 20% increase in the level of the exhaled miRNA, optionally at least 50% increase in the level of the exhaled miRNA.

22. The method of 19, wherein said significantly lower level of the exhaled miRNA comprises an at least 20% decrease in the level of the exhaled miRNA, optionally at least 50% decrease in the level of the exhaled miRNA.

23. The method of 15 or 16, wherein the control is a miRNA level in the EBC or a derivative thereof from a healthy subject.

24. The method of any one of 15-23, further comprising recommending, prescribing, and/or administering to the subject a therapy (e.g., a cancer therapy).

25. A method of preventing or treating a disease in a subject, the method comprising administering to the subject a therapy, wherein the subject is determined to be in need of a therapy according to the method of any one of 14-22.

26. The method of any one of 1-24, wherein the subject is healthy, the subject is afflicted with a lung disease, or the subject is at risk for developing a lung disease.

27. The method of any one of 15-26, wherein the lung disease is a cancer (e.g., an early lung cancer), asthma, or chronic obstructive pulmonary disease (COPD).

28. The method of any one of 16, 18, 19, 24, and 25, wherein the therapy treats a cancer, asthma, or COPD.

29. The method of 27 or 28, wherein the cancer is adenocarcinoma, squamous cell carcinoma, small cell lung cancer (SCLC), non-small cell lung cancer (NSCLC), or metastases from another organ to a lung.

30. The method of 28, wherein the therapy comprises a surgery, chemotherapy, cancer vaccines, chimeric antigen receptors, radiation therapy, immunotherapy, a modulator of expression of immune checkpoint inhibitory proteins or ligands, corticosteroids, leukotriene modifiers, combination inhalers, theophylline, beta agonists, anticholinergic agents, bronchodilators, steroids, phosphodiesterase-4 inhibitors, antibiotics, or any combination thereof.

31. The method of any one of 1-30, wherein the subject is a mammal.

32 The method of any one of 1-31, wherein the subject is a mouse or a human.

33. The method of any one of 1-32, wherein the subject is a never smoker, former smoker, or a current smoker.

34. The method of any one of 1-33, wherein the subject has an underlying lung disease, optionally wherein the underlying lung disease is selected from chronic obstructive pulmonary disease (COPD), fibrosis, inflammation, asthma, sarcoidosis, and bronchiectasis.

### Examples

### Example 1: Materials and Methods

### EBC Donor Recruitment and Sample Collection

### Subject Recruitment

A series of 351 consenting individuals destined for invasive lung sampling for clinical purposes (bronchoscopy or thoracic surgery) were enrolled under a protocol approved by the Einstein-Montefiore institutional review board (IRB). This observational series work was PROBE compliant. This study included 166 cases of lung cancer and 185 controls without lung cancer (Table 1). It also included 4 healthy lab volunteers; EBC was collected from every volunteer at three different timepoints (0, 24, and 96 hours). EBC (and other non-invasive airway specimen) collection occurred immediately prior to the planned bronchoscopy/thoracic surgery, to preclude procedure-induced spillage of lung materials into the EBC (and mouthwash) samples. Clinical data was obtained by direct interview in advance of any clinically-indicated bronchoscopic/surgical procedure (and therefore in advance of tissue diagnosis), and verified manually in the clinical electronic medical record. Inclusions were: age>21; fitness for the clinically-indicated (bronchoscopy/surgical) procedure; capacity and willingness to consent. Exclusions were: acute respiratory illness, contraindications to additional brushings/bronchoalveolar lavage (coagulopathy/known poorly controlled uremia); lack of capacity for consent. As such, subjects entailed a diversity of ages, ethnicities, smoking histories, clinical diagnoses, and underlying chronic lung diseases, which were accounted for in the models.

### EBC Sample Collection

The EBC collection followed the recommendations of the American Thoracic Society/European Respiratory Society Task Force on EBC. RTube™ (Respiratory Research, Inc) was used to collect patient's Exhaled Breath condensate (EBC), per standard protocol. The essentials of the simple RTube® device are (i) One way inhalation/exhalation valve; (ii) Small port for exhaled breath mixing and turbulence; (iii) Exhalation cooling chamber, polypropylene; (iv) Manually operated piston for condensate capture. Briefly, before any clinically-indicated lung procedure, subjects were equipped with RTube/mouthpiece/noseclips and performed quiet, tidal volume breathing plus one deeper breath (sigh) per minute, collected over a 10-15 minute span, while seated; saliva was to be swallowed, and excess saliva was trapped by RTube® device by design. Any coughing was instructed to be done off of the mouthpiece, to minimize oral contamination. A bare minimum of 100 ul of EBC was the goal, and achieved in >75% of subjects. Over 50% of individuals collected >500 ul EBC.

### Exosome Partitioning Using EV-CATCHER

In some experiments, EV-CATCHER® was used to partition small EVs comprising miRNAs prior to RNA extraction (see e.g., FIG. 12) (EV-CATCHER® is described in US2022/0205990 and WO2022/140662, each of which is incorporated herein by reference).

HPLCpurified uracilated oligonucleotides (Integrative DNA Technology) for the 5′-Azide (5′Az-AAAAACGAUUCGAGAACGU GACUGCCAUGCCAGCUCGUACUAU CGAA) and 3′-Biotin (5′Bio-CGAUAGUACGAGCUGGCAUGGCAGUCACGUUC UCGAAUCGUUUU), adapted from Löf et al. (2017) were resuspended in RNase-free water at a concentration of 250 ng/μl. Equimolar amounts (1:1 ratios) of each oligo were annealed (90° C. for 2 min, 90-42° C. for 40 min, 42° C. for 120 min) in 1×RNA annealing buffer (60 mM KCl, 6 mM HEPES (pH 7.5), 0.2 mM MgCl2), prior to separation on a 15% non-denaturing polyacrylamide (PAGE) gel (0.5×TBE (ThermoFisher, #15581044) at 450 volts for 90 min). The double stranded (ds) DNA linker was visualized on a blue light box with SYBR Gold dye (ThermoFisher, #S11494), excised, centrifugally crushed using a gel breaker tube (IST Engineering, #3388-100) and resuspended in 400 mM NaCl and shaken overnight (O/N) on a thermomixer set to 4° C. and 1,100 RPM. The solution was filtered, and the dsDNA linker was purified using the QIAEX® II gel extraction kit (Qiagen, #20021) according to manufacturer instructions. Purified dsDNA linker was evaluated on a NanoDrop 2000 and diluted to 250 ng/μl. Antibodies (1 mg/ml) used for exosome pulls (anti-CD63 (Abcam, #ab59479), anti-CD81 (Abcam, #ab233692) and anti-CD9 (Abcam, #ab263023)) were activated using 5 μl of freshly prepared 4 mM DBCO-S-S-NHS ester (Sigma Aldrich, #761532) and incubated for 30 min at room temperature (RT) in the dark, reactions were stopped by adding 2.5 μl of 1 M Tris-Cl (pH 8.0) at RT for 5 min in the dark. DBCO-activated antibodies were desalted onto pre-equilibrated Zeba desalting columns (ThermoFisher, #89882) by incubation for 1 min and centrifugation at 1500×g for 2 min. Antibodies were quantified on a Nanodrop 2000 instrument using protein A280 and antibody-dsDNA (Ab-dsDNA) stock solutions were prepared by conjugating 50 μg of activated antibody with 25 μg of purified DNA linker, overnight at 4° C. on a rotator. Validation of Ab-dsDNA binding was performed by PAGE where the Ab-dsDNA (1 μg) product was run under non-denaturing and non-reducing conditions, followed by Coomassie (Bio-Rad #1610786) staining to visualize the shift in Ab-dsDNA migration. The next day, Ab-dsDNA conjugates were bound to streptavidin coated 96-well plates (Pierce, #15120). Either single anti-CD63 antibody (1 μg) or a combination of anti-CD63, -CD81 and -CD9 (1 μg of each antibody) (linker bound) was added to single wells in 100 μl 1×PBS. Streptavidin coated 96-well plates with Ab-dsDNA conjugates were placed on a plate shaker at 300 RPM at 4° C., for 8 h to allow for binding to the plate. Solutions were carefully removed, and wells were washed three times with cold 1×PBS solution, prior to addition of RNase-A (12.5 μg/ml) treated samples (100 μl). Plates were sealed using microAMP optical adhesive film (Applied Biosystems, #4311971) and placed on a shaker at 300 RPM at 4° C., O/N. Samples were carefully removed, wells were washed 3 times with cold 1×PBS and 100 μl of freshly prepared uracil glycosylase (UNG) enzyme (ThermoFisher, #EN0362) in 1×PBS (1×UNG buffer (200 mM Tris-Cl (pH 8.0), 10 mM EDTA and 100 mM NaCl), with 1 unit of enzyme) was added to each well. Plates were incubated at 37° C. for 2 h on a shaker at 300 RPM for UNG digest of the dsDNA linker, and small-EVs were recovered in this solution for further characterization and downstream analyses.

Optimization of EBC microRNA Extraction

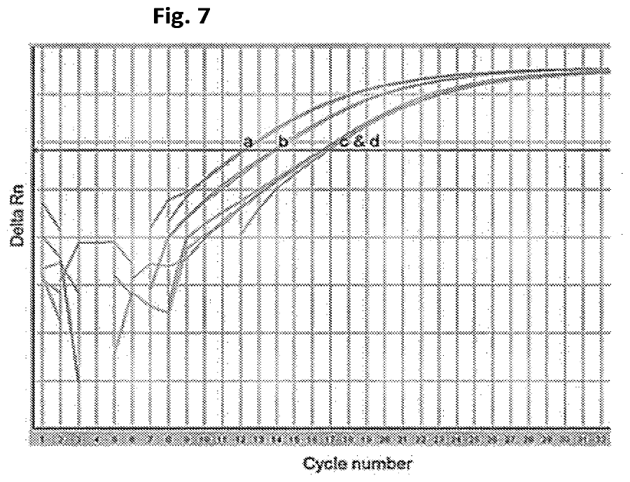

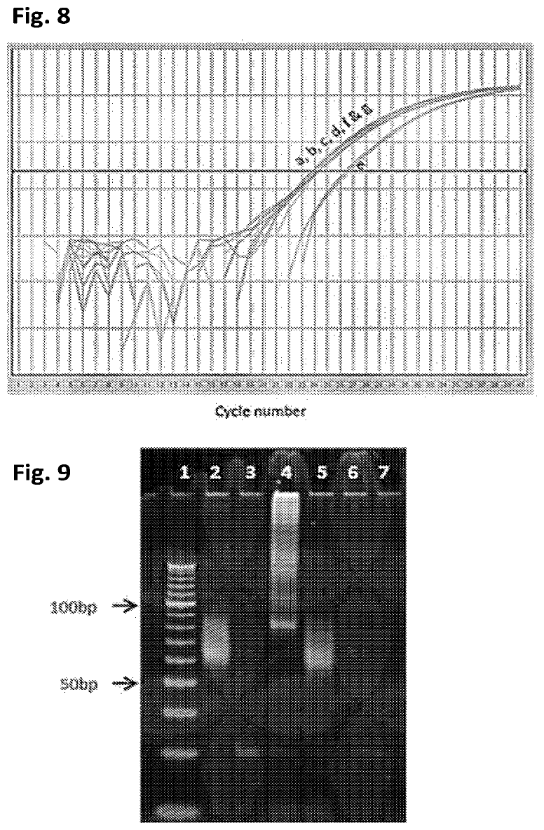

Optimization of EBC microRNA extraction was performed, comparing ethanol alone, trizol alone, speed vacuum alone, column-based method alone (FIG. 7) and combinations of the above with glycogen and carrier RNA (FIG. 8). Optimal was ethanol precipitation with glycogen carrier molecule, and the final protocol included a biochemical/Trizolbased isolation.

### RNA Extraction

For total RNA extraction, EBC was concentrated by ethanol precipitation and then was purified by Trizol (Invitrogen) per manufacturer protocol and lab optimized protocol. The following components were added into a capped polypropylene tube and thoroughly mixed, including 100-400 ul of EBC sample, 40 ul of 3M sodium acetate (pH 5.5), 5 ul of 5 ug/ul glycogen carrier, and 1100 ul of 100% cold ethanol. The mixture was chilled at −80° C. for 30 min and then centrifuged at 14,000 rpm for 20 min at 4° C. Then, the supernatant was discarded and the pellet was rinsed with cold 70% ethanol twice, and air-dried. The pellet was then dissolved in 0.5 ml of Trizol®. Total RNA was purified per the Trizol® manufacturer protocol. The RNA pellet was dissolved in 15 ul of RNase-free water.

MicroRNA Specificity Testing of Newly Designed microRNA PCR Primers

All microRNA primers were initially tested for microRNA specificity on RNA extracts from lung cell lysates, and on pilot EBC samples, using both realtime RT-qPCR and gel electropheresis with key controls. This testing included a: (a) no-RT step (to exclude gDNA-derived signal); (b) no polyA-step (to exclude messenger RNA-derived signal); (c) genomic DNA spike-in (to exclude gDNA-derived signal); and (d) water-no template blank (to exclude cDNA or PCR-product contaminated reagents). Cell culture samples were used as positive controls for EBC microRNA-PCR development: For positive controls in microRNA-PCR assay development, total RNA extracts from a set of pooled cell lines including NHBE, HBEC, A549, Hela, HTB-119 and CRL-1995 was combined and RNA extracted in conventional column (RNeasy, Qiagen). This provided a stock solution of total RNA for initial testing of microRNA-specific primers.

Universal RT Primer Optimization for microRNAs

For microRNAs, the big issue of universal RT primer is that it can often amplify similar size products from both cDNA with polyadenylation and cDNA without polyadenylation. Every microRNA primerset will be tested on two different templates (cell cDNA with polyadenylation and cell cDNA without polyadenylation). The microRNA primers sequences used in the study are listed in Supplemental Table 8.

### MicroRNA PCR Analysis

The overall strategy was to amplify mature microRNAs by a previously published lab protocol involving poly-A tailing using a one-base anchored and universal tagged oligo-dT-RT strategy, and a microRNA-specific forward primer coupled to a universal, human-unique tag-specific reverse primer, in aggregate precluding false gDNA amplification. Individual steps and details follow.

Cell Culture Samples Used for microRNA PCR Development

For positive controls in microRNA-PCR assay development, a set of cell lines including NHBE, HBEC, A549, Hela, HTB-119 and CRL-1995 was RNA extracted in conventional column (RNEasy, Qiagen), and provided a stock solution of total RNA for initial testing of microRNA-specific primers.

### Poly(A) Tailing

The Poly(A) Tailing Kit (Ambion) was used to polyadenylate the 3′ termini of microRNA. First, ATP was diluted to 1% of the original concentration. Then, the following components were added into a PCR tube and thoroughly mixed, including 2 ul of 5× buffer, 0.8 ul of MnCl₂ (25 mM), 0.4 ul of diluted ATP, 0.25 ul of enzyme and 6.55 ul of total RNA from EBC. The mixture was incubated at 37° C. for 30 min.

### Reverse Transcription

Reverse transcription was performed with 10 μl of the E. coli Poly(A) Polymerase (E-PAP) treated total RNA using Superscript III reverse transcriptase (Invitrogen) as follows. RNA template was added to a master mix containing 1 μl of 100 μM universal oligo-dT-adapted universal RT primer, 1 μl of dNTP mix (each base 10 μM) and 1 μl of DNase/RNase-free water. Total volume was adjusted to 13 μl with DNase/RNase-free water. The solution was incubated at 65° C. for 5 min and then cooled on ice. A master mix containing 4 μl of 5× first-strand buffer, 1 μl of 0.1 mM DTT, 1 μl RNaseOUT (Invitrogen) and 1 μl SuperScript III per RT sample was prepared and added to each sample. The samples were incubated at 42° C. for 30 min, 50° C. for 30 min, followed by 70° C. for 15 min.

### Realtime PCR

Typically, the RT reaction was diluted 1:20 and 2 μl used in the realtime PCR of microRNAs with the transcript specific forward PCR primers (Supplemental data, Table 8, n=25 primersets) and a matched (tag-directed) reverse primer. 25 microRNA candidates were from our pilot study of lung tissue-based tumor versus non-tumor discriminant microRNAs and the literatures, as described in the background part. Our pilot study can be found in the supplemental data. cDNA template was added to a master mix containing 10 ul of 2× PowerSYBR green master mix (Applied Biosystem), 1 ul of 10 uM primers mix and 7 ul of DNase/RNase-free water. The reaction was incubated in an Applied Biosystems 7500 realtime PCR system at 95° C. for 10 min, followed by 45 cycles of 95° C. for 15 s, 60° C. for 15 s and 72° C. for 32 s. After that, dissociation stage/melting curve analysis was performed. In developing each primerset, primers were designed to produce a single unique melting curve on known microRNA extracts from lung cell lines. Multiple separate positive and negative controls in both lung cell lines and EBC sample standards were run, including (a) gDNA spike (to exclude false gDNA amplification) (b) no-RTase (to exclude false gDNA amplification); (c) no poly Adenylation (to exclude false messenger RNA amplification); (d) no template (to exclude reagent contamination by PCR product).

### Data Cleaning Scoring

Since microRNAs are all of near-identical size, base composition/melting temperature was a major distinguishing feature. The criteria for including or excluding a micro-RNA-derived PCR product as present were extracted from the melting curves. If a sample had the same melting curve maximum temperature (Tm) as the positive control from cell lines for that microRNA primerset, it was called “positive”. If a reaction sample had no visible melting curve, or the visible melting curve displayed greater than +/−1.5° C. different Tm from the melting curve from the positive, individual miR-specific control, it was called “negative”. We used one convention for overall scoring of samples—at least one of two replicates must be positive. The housekeeper control chosen, based on literature, and ubiquitous presence in our EBC samples, was miR-423-3p. From previously described studies, hsa-miR-16, hsa-miR-26b, hsa-miR-92, hsa-miR-423, hsa-miR-374, are often used as housekeeper controls.

### URT-PCR Versus Taqman PCR

In order to compare URT-PCR and Taqman PCR, let-7a, let-7f., miR-18a, miR-21, miR-26a, miR-140, miR-212, miR-423-3p, miR-708 and miR-767 were chosen as arbitrary targets (Table 10). Samples chosen were 100 ng/ul of HBEC total RNA, 1 ng/ul of HBEC total RNA and RNA of EBC. RNA of EBC was purified by the optimized miRNA extraction method in this study. URT-PCR was performed by our previously published URT-PCR lab protocol and Taqman PCR was performed by Invitrogen Taqman miRNA assays according to TaqMan-designed primers and the manufacturers protocol. For URT-PCR, controls included cDNA of miRNA, no RT (omitted RTase), cDNA of mRNA (no poly-adenylase), genomic DNA spikein, and water blank (no template). MiR-423-3p was used as a housekeeper, though it was not detected by TaqMan system. During URT-PCR analysis, melting curves were examined in order to verify correct PCR products.

### Statistical Analysis

### Logistic Regression (LR)

Logistic Regression was performed for each miRNA with cancer case-control status as the response, with and without the clinical variables included as the covariates. The clinical covariates are age, gender, smoking status (never smokers, former smokers, current smokers), pack years, quit years, and underlying lung diseases (categorized in three groups, (1) any of COPD, fibrosis, generic inflammation and/or asthma; (2) sarcoid and bronchiectasis; (3) none and others.

### Random Forests (RF)

Two types of Random Forest classifiers were built for comparison, using R package random forest. First, an RF classifier was built on the clinical variables alone: age, gender, smoking status, pack-years, quit-years, underlying lung disease (type), tumor histology, stage. Two-fold cross-validation was repeated 20 times to gauge the accuracy of this classifier, and its sensitivity, specificity, positive and negative predictive value. Second, an RF classifier was built on the clinical variable plus the microRNA variables together. To compare the performance of the two types of RF classifiers, we further generated 100 resampled ROC curves for each one and compared the average area under the curve (AUC) between the two models using a two-independent sample t-test. A resampled ROC curve was generated by repeatedly splitting the dataset into 50% training, 50% testing (100 times), building the two random forest models (clinical and clinical+microRNA), and predicting the outcomes of the testing split.

### Airway Topography Similarity Statistic

A subset of 12 EBC donors provided bronchoscopic samples of deep alveolar (BAL) and major airway (bronchial, BB) levels, as well as sputum, mouthrinse and other specimens. The pilot sub-study (Table 9) was designed to evaluate if an individual microRNA profile from EBC retains the distinct features of the microRNA profile from deep lung (bronchial brushings or bronchoalveolar lavage), or alternately resembles contaminating upper airway/mouthwash tissues. This was done by applying an arbitrary panel of 13-microRNAs interrogated by qualitative RT-PCR against samples from 12 individuals, each donating five airway level samples for comparison [bronchoalveolar lavage (BAL), bronchial brush (BB), sputum (SP), mouthrinse (MW), EBC]. To statistically test the surrogacy of EBC-microRNA for deeper lung specimens (bronchial brushings and bronchoalveolar lavage), we developed a similarity statistic of two tissue types based on Hamming distance. That is SH=Σᵢ H(dᵢ, d′ᵢ), where dᵢ and d′ᵢ are (binary) miRNA profiles from two tissue types of the same individual i. The Hamming distance H gives the total number of miRNAs for which the two profiles d and d′ are discordant. The smaller the statistic SH is, the more similar are two tissue types in miRNA profiles within each subject. If the two tissue types from the same individual are not closer than two tissue types from two random individuals, then there is no information in one of the tissues to infer the miRNA profile of the other tissue. To test that the two tissues from the same individuals are closer than two random individuals, we performed a permutation test that permutes the miRNA profiles within each tissue type among individuals.

Temporal Stability of EBC miRNA for an Individual Across Time

The EBC samples from 4 healthy lab volunteers at three different timepoints were used to evaluate temporal stability of EBC miRNA for an individual across time. The three targets were miR-141, miR-142-3p and miR-205 and the housekeeping gene is miR-423-3p. Heat matrix was built by delta Ct (normalize Ct of target miRNA to Ct of housekeeping miR-423-3p). The total qPCR cycles was 40. For the housekeeping miR-423-3p, the Ct cutoff was 35 and the melting curve of SYBR green qPCR must be correct.

### Example 2: The Clinical Characteristics of the 351 Subjects

The clinical characteristics of the 351 subjects are described in Table 1. Baseline clinical characteristics that differed between cases and controls included age, smoking status, pack-years, quit years, underlying lung disease. Former smokers were defined as quit greater than one year from enrollment. Cases significantly differed from controls for: age (66.9 vs. 56.4, resp.); smoking status (current 43.4 vs. 20.5%, former 47.6% vs. 41.6%, never 9.0 vs. 37.8% never smokers); pack-years among current/former smokers (43.4 vs. 19.2); quit years among former smokers (7.3 vs. 9.4), pack years-quit years index (31.1 vs. 5.8); Underlying lung disease including COPD % (56.0 vs. 19.5); inflammation NOS % (1.2 vs. 10.3); sarcoidosis % (1.2 vs 8.6); none % (31.8 vs. 49.2). Both logistic regression (LR) and random forest (RF) discriminant models took these clinical inter-group differences into account. For RF, this included measuring the incremental impact on case-control discrimination of microRNAs over and above these clinical factors alone.

TABLE 1
Clinical characteristics among 351 cases and controls

| | Cases | Controls | | |
|---|---|---|---|---|
| | (n = 166) | (n = 185) | Statistics | p-value |
| Age (years) | 66.93 | 56.40 | t-test | 1.23E−15 |
| Gender (% male) | 48.80 | 49.73 | chi-square | 0.86 |
| Smoking Status (%) | overall | | chi-square | 1.51E−10 |
| Current | 43.37 | 20.54 | | |
| Former | 47.59 | 41.62 | | |
| Never | 9.04 | 37.84 | | |
| Pack Years | 43.43 | 19.21 | t-test | 3.12E−09 |
| Quit Years | 7.33 | 9.40 | t-test | 6.00E−03 |
| (former smokers) | | | | |
| Pack years-Quit Years | 31.14 | 5.82 | t-test | 7.32E−06 |
| Tumor Histology (%) | | | N/A | |
| Adeno | 50.0 | N/A | | |
| Squam | 21.1 | N/A | | |
| Undiff NSCLC | 15.7 | N/A | | |
| Small Cell | 9.0 | N/A | | |
| Mets/Other | 4.2 | N/A | | |
| Stage (%) | | | N/A | |
| I | 33.13 | N/A | | |
| II | 12.05 | N/A | | |
| III | 31.33 | N/A | | |
| IV | 11.45 | N/A | | |
| ULD (%) | | | | |
| COPD | 56.02 | 19.46 | Fisher | 1.29E−12 |
| Fibrosis | 1.20 | 2.16 | Fisher | 0.69 |
| Inflammation, NOS | 1.20 | 10.27 | Fisher | 2.16E−04 |
| Asthma | 12.05 | 16.76 | Fisher | 0.23 |
| Sarcoid | 1.20 | 8.65 | Fisher | 1.30E−03 |
| Bronchiectasis | 2.41 | 2.70 | Fisher | 1.00 |
| None | 31.33 | 49.19 | Fisher | 7.00E−04 |

Clinical characteristics of the case versus control subjects. Former smoker, defined as quit >1 year; COPD, defined clinically (MD report, medications), radiographically, and/or pathologically in medical records; Pack-yrs-Quit-yrs, in former smokers, a constructed variable combining cumulative dose (pack years) minus proximity of smoking (quit-years); NOS, not otherwise specified. Adeno-adenocarcinoma; Squamous=squamous cell carcinoma; NSCLC-Undifferentiated non-small cell lung cancer; Small cell=small cell carcinoma; Mets/Other=metastases from other organs to lung or other tumor histologies. ULD-Underlying (chronic) lung disease.

### Example 3: EBC Surrogacy for the Lung

The similarity statistic of two tissue types that were based on Hamming distance, SH=Σᵢ H(dᵢ, d′ᵢ), and 1000 permutations of miRNA profiles within each tissue type among individuals, gave an estimated P-value of 0.007, suggesting that the miRNA profiles of EBC are closer to miRNA profiles of BAL of the same individual than to miRNA profiles of BAL of random individuals. The same analysis was applied between EBC and BB (p=0.23), EBC and SP (p=0.18), EBC and MW (p=0.04).

### Example 4: Logistic Regression (LR)

LR models were created (Table 2), using individual exhaled microRNA presence or absence as univariate predictors of case-control status, with adjustment for clinical factors: age, gender, smoking status (current, former, never), smoking pack years and quit years, and presence of underlying lung disease. For the entire data set, miR-21, 33b and 212 appeared to be somewhat informative for case-control status (p<0.05), after adjustment for the above-listed clinical factors.

TABLE 2
Logistic regression, univariate miR, All subjects, n = 351

| ## | miRNA | p | p. adj |
|---|---|---|---|
| 1 | miR.324.5p | 0.967 | 0.817 |
| 2 | miR.9 | 0.383 | 0.779 |
| 3 | miR.21 | 0.090 | 0.020 |
| 4 | miR.31 | 0.343 | 0.231 |
| 5 | miR.33b | 0.011 | 0.017 |
| 6 | miR.96 | 0.631 | 0.342 |
| 7 | miR.105 | 0.677 | 0.142 |
| 8 | miR.146a.5p | 0.358 | 0.340 |
| 9 | miR.182.5p | 0.840 | 0.687 |
| 10 | miR.196b | 0.385 | 0.587 |
| 11 | miR.199b.5p | 0.640 | 0.562 |
| 12 | miR.200a | 0.799 | 0.396 |
| 13 | miR.200b | 1.000 | 0.959 |
| 14 | miR.205 | 0.664 | 0.793 |
| 15 | miR.212 | 0.153 | 0.033 |
| 16 | miR.221 | 0.932 | 0.386 |
| 17 | miR.345 | 0.113 | 0.081 |
| 18 | miR.429 | 1.000 | 0.601 |
| 19 | miR.767 | 0.236 | 0.476 |
| 20 | miR.944 | 0.053 | 0.404 |
| 21 | miR.1269a | 0.059 | 0.496 |
| 22 | miR.1293 | 0.102 | 0.230 |
| 23 | miR.1910 | 0.261 | 0.154 |
| 24 | miR.3662 | 0.862 | 0.539 |

The univariate models included adjustments for clinical factors of age, gender, smoking status (current, former, never), smoking pack-years and quit-years, and underlying lung disease. Underlying lung disease: For all models, underlying lung disease was treated as trichotomous (COPD/fibrosis//inflammation NOS, asthma) versus sarcoidosis/bronchiectasis) versus none/other. Housekeeper miR423-5p, not listed.

### Example 5: Random Forests

Clinical, exhaled microRNA, and combined clinical+exhaled microRNA RF models discriminating cases from controls were constructed (Table 3). For lung cancer overall, including all subjects (n=351) and all case primary lung malignant tumor histologies, the clinical RF model included age, gender smoking status, pack-years, quit-years, underlying lung disease. For the clinical only RF model alone, case-control discriminant accuracy, sensitivity, specificity, positive predictive value, negative predictive value, AUC-ROC, were: 0.74, 0.74, 0.74, 0.76, 0.72, 0.814, respectively. For the microRNAs only model, the respective values were: 0.57, 0.63, 0.50, 0.58, 0.55, 0.611. For the combined clinical+microRNA model, the respective performance values were: 0.74, 0.74, 0.74, 0.76, 0.73, 0.826. The added AUC discrimination conferred by exhaled microRNAs for the overall group of subjects (n=351) was 1.2% (0.814=>0.826; p=0.07, Welch t-test).

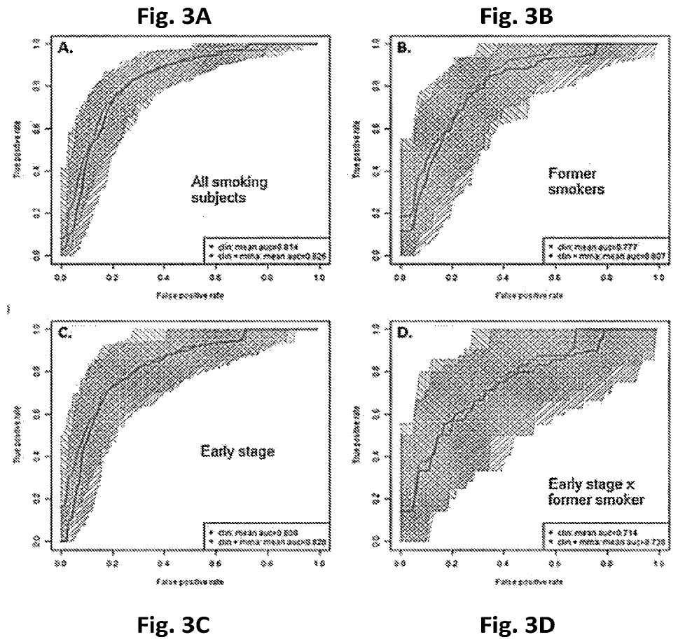

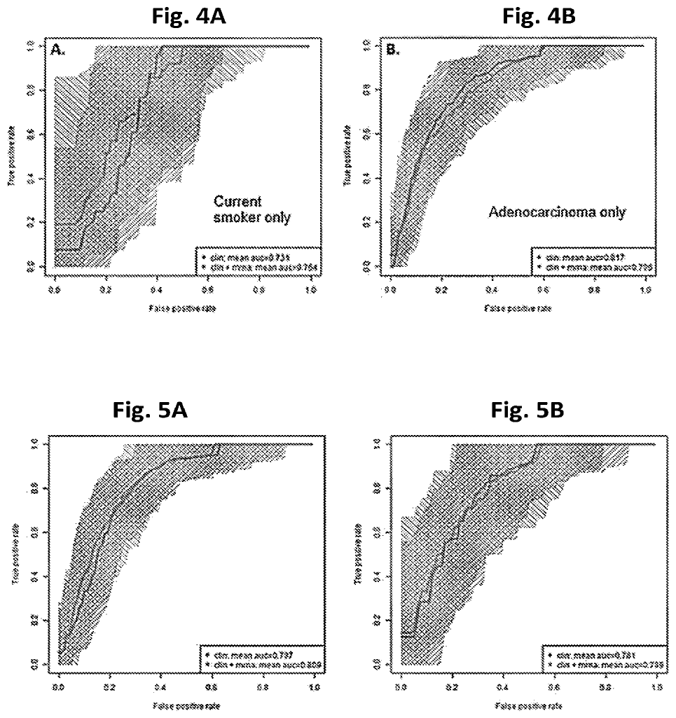

For a priori selected subgroups, data analyses are also tabulated (Table 3). For example, Former smoker cases versus former smoker controls comparison of case-control discriminant performance were again described in terms of discriminant accuracy, sensitivity, specificity, positive predictive value, negative predictive value, AUC-ROC. For the clinical only model, performance parameters were: 0.69, 0.69, 0.69, 0.69, 0.70, 0.777, respectively. For the microRNA-only model, performance were: 0.59, 0.57, 0.61, 0.59, 0.59, 0.656, respectively. For the combined clinical+microRNA model, performance were: 0.70, 0.67, 0.72, 0.70, 0.69, 0.807, respectively. The added AUC discrimination conferred by exhaled microRNAs was 3.0% (0.777=>0.807; p-6.0e-03, Welch t-test) for former smokers. Similarly, early stage (I+II combined) cases showed 2.2% added case-control AUC discrimination from the exhaled microRNA panel (p=5.1e-03) (FIG. 3C). For additional clinically important combined subgroups, the case versus controls models' performance characteristics are described in Table 3, FIG. 3A-FIG. 3D, FIG. 4A-FIG. 4B, and FIG. 5A-FIG. 5B.

TABLE 3
Exhaled microRNA RF models

| | | | | | | AUC |
|---|---|---|---|---|---|---|
| | | | | | | difference, |
| | | | | | | Clinical vs |
| | | | | | | Clinical + |
| RF models, | Individual | | | | | micro- |
| lower | component | Accuracy | Sensi, | PPV, | ROC- | RNA, % |
| stringency | factors | (p-value) | Speci | NPV | AUC | (p-value) |
| All subjects, all | | | | | | |
| smoking categories, | | | | | | |
| all tumor | | | | | | |
| histologies, n = 166 | | | | | | |
| cases, 185 controls | | | | | | |
| Clinical Variables | All clinical | 0.74 | 0.74, | 0.76, | 0.814 | |
| Alone (unselected) | variables [age, | (<2.2e−16) | 0.74 | 0.72 | | |
| | gender, smoking | | | | | |
| | status, pack-years, | | | | | |
| | quit-years, | | | | | |
| | underlying lung | | | | | |
| | disease, tumor | | | | | |
| | histology for cases] | | | | | |
| microRNAs alone | All 24 microRNAs. | 0.57 | 0.63, | 0.58, | 0.611 | |
| | Important miRs: | (<2.2e−16) | 0.50 | 0.55 | | |
| | 21, 33b, 944, | | | | | |
| | 1269a, 1910. | | | | | |
| Clinical + microRNA | All Clinical factors | 0.74 | 0.74, | 0.76, | 0.826 | 1.2% |
| | and All 24 miRs | (2e−16) | 0.74 | 0.73 | | (0.07) |
| Former smokers only | | | | | | |
| n = 79cases, 77 | | | | | | |
| controls | | | | | | |
| Clinical Variables | All clinical | 0.69 | 0.69 | 0.69 | 0.777 | |
| Alone | variables [age, | (<2.2e−16) | 0.69 | 0.70 | | |
| | gender, smoking | | | | | |
| | status, pack-years, | | | | | |
| | quit-years, | | | | | |
| | underlying lung | | | | | |
| | disease] | | | | | |
| microRNAs alone | All 24 microRNAs | 0.59 | 0.57 | 0.59 | 0.656 | |
| | Important miRs: | (<2.0e−16) | 0.61 | 0.59 | | |
| | 33b, 146a.5p, 200a, | | | | | |
| | 212, 1293. | | | | | |
| Clinical + microRNA | All Clinical and All | 0.70 | 0.67 | 0.70 | 0.807 | 3.0% |
| | miRs | (<2.0e−16) | 0.72 | 0.69 | | (6.0e−03) |
| Early Stage only | | | | | | |
| (stages I and II) | | | | | | |
| n = 78 cases, 184 | | | | | | |
| controls | | | | | | |
| Clinical Variables | All clinical | 0.75 | 0.86 | 0.80, | 0.806 | |
| Alone | variables [age, | (2.2e−16) | 0.50 | 0.60 | | |
| | gender, smoking | | | | | |
| | status, pack-years, | | | | | |
| | quit-years, | | | | | |
| | underlying lung | | | | | |
| | disease] | | | | | |
| microRNAs alone | All 24 microRNAs. | 0.700 | 0.90, | 0.73 | | |
| | Important miRs: | (NS) | 0.23 | 0.49 | | |
| | 96, 146a.5p, 944, | | | | | |
| | 1269a, 1910. | | | | | |
| Clinical + microRNA | All Clinical | 0.76 | 0.90, | 0.79 | 0.828 | (2.2% |
| | variables and All | (<2.2e−16) | 0.45 | 0.65 | | (5.1e−03) |
| | miRs | | | | | |
| Former Smoker × | | | | | | |
| Early Stage Sub- | | | | | | |
| subgroup n = 34 | | | | | | |
| cases, 77 controls | | | | | | |
| Clinical Variables | All clinical | 0.71 | 0.87 | 0.75 | 0.714 | |
| Alone | variables [age, | (1.9e−03) | 0.35 | 0.54 | | |
| | gender, smoking | | | | | |
| | status, pack-years, | | | | | |
| | quit-years, | | | | | |
| | underlying lung | | | | | |
| | disease, tumor | | | | | |
| | histology for cases] | | | | | |
| microRNAs alone | All 24 microRNAs. | 0.67 | 0.86 | 0.72 | 0.641 | |
| | Important miRs: | (NS) | 0.25 | 0.44 | | |
| | 96, 146a.5p, 200b, | | | | | |
| | 345, 1910 | | | | | |
| Clinical + microRNA | All Clinical and All | 0.71 | 0.90 | 0.74 | 0.738 | 2.4% |
| | miRs | (1.4e−02) | 0.28 | 0.54 | | (NS) |
| Current Smokers | | | | | | |
| only, n = 38cases, 72 | | | | | | |
| controls | | | | | | |
| Clinical Variables | All clinical | 0.78 | 0.59 | 0.71 | 0.731 | |
| Alone | variables [age, | (2.2e−16) | 0.87 | 0.80 | | |
| | gender, smoking | | | | | |
| | status, pack-years, | | | | | |
| | quit-years, | | | | | |
| | underlying lung | | | | | |
| | disease, tumor | | | | | |
| | histology for cases] | | | | | |
| microRNAs alone | All 24 microRNAs. | 0.59 | 0.15 | 0.30 | 0.464 | |
| | Important miRs: | (NS) | 0.82 | 0.65 | | |
| | 105, 146a.5p, | | | | | |
| | 182.5p, 200a, 205. | | | | | |
| Clinical + microRNA | All Clinical and All | 0.76 | 0.48 | 0.73 | 0.764 | 3.3% |
| | miRs | (2.2e−16) | 0.90 | 0.77 | | (3.5e−02) |
| Adenocarcinoma | | | | | | |
| only, n = 87cases, 184 | | | | | | |
| controls | | | | | | |
| Clinical Variables | All clinical | 0.74 | 0.83 | 0.79 | 0.817 | |
| Alone | variables [age, | (<2.2e−16) | 0.53 | 0.60 | | |
| | gender, smoking | | | | | |
| | status, pack-years, | | | | | |
| | quit-years, | | | | | |
| | underlying lung | | | | | |
| | disease] | | | | | |
| microRNAs alone | All 24 microRNAs. | 0.65 | 0.87 | 0.69 | 0.579 | |
| | Important miRs: | (NS) | 0.18 | 0.40 | | |
| | 96, 146a.5p, 221, | | | | | |
| | 944, 1269a | | | | | |
| Clinical + microRNA | All Clinical and All | 0.73 | 0.87 | 0.76 | 0.796 | −2.1% |
| | miRs | (2.2e−16) | 0.43 | 0.61 | | (1.1e−02), |
| | | | | | | neg |
| | | | | | | direction |
| Late Stage (III, IV) | | | | | | |
| only, n = 77cases, 184 | | | | | | |
| controls | | | | | | |
| Clinical Variables | All clinical | 0.74 | 0.84 | 0.80 | 0.797 | |
| Alone | variables [age, | (2.2e−16) | 0.49 | 0.56 | | |
| | gender, smoking | | | | | |
| | status, pack-years, | | | | | |
| | quit years, | | | | | |
| | underlying lung | | | | | |
| | disease, underlying | | | | | |
| | lung disease] | | | | | |
| microRNAs alone | All 24 microRNAs. | 0.67 | 0.90 | 0.71 | 0.541 | |
| | Important miRs: | (NS) | 0.12 | 0.35 | | |
| | 31, 33b, 105, 212, | | | | | |
| | 944 | | | | | |
| Clinical + microRNA | All Clinical and All | 0.74 | 089 | 0.77 | 0.809 | 1.2% |
| | miRs | (2.2e−16) | 0.38 | 0.58 | | (NS) |
| Late Stage (III, IV) × | | | | | | |
| Former Smoker, n = | | | | | | |
| 40 cases, 77 controls | | | | | | |
| Clinical Variables | All clinical | 0.70 | 0.82 | 0.75 | 0.781 | |
| Alone | variables [age, | (1.6e−13) | 0.46 | 0.57 | | |
| | gender, smoking | | | | | |
| | status, pack-years, | | | | | |
| | quit years, | | | | | |
| | underlying lung | | | | | |
| | disease, underlying | | | | | |
| | lung disease] | | | | | |
| | histologies?] | | | | | |
| microRNAs alone | All 24 microRNAs. | 0.63 | 0.84 | 0.67 | 0.654 | |
| | Important miRs: | (NS) | 0.22 | 0.41 | | |
| | 33b, 200a, 212, | | | | | |
| | 345, 1293 | | | | | |
| Clinical + microRNA | All Clinical and All | 0.69 | 0.85 | 0.72 | 0.789 | 0.8% |
| | miRs | (1.1e−07) | 0.37 | 0.57 | | (NS) |

RF Models of case-control distinction, including clinical variables alone, exhaled microRNAs alone, and the two combined, and performance characteristics of accuracy, sensitivity, specificity, positive and negative predictive value (PPV, NPV, resp.), Underlying lung disease is treated as a (trichotomous) variable: (3). COPD or fibrosis or inflammation or asthma (all carry some lung cancer risk); versus (2). sarcoid bronchiectasis; versus (1) none/other).

### Example 6: Temporal Stability of EBC miRNA for an Individual Across Time

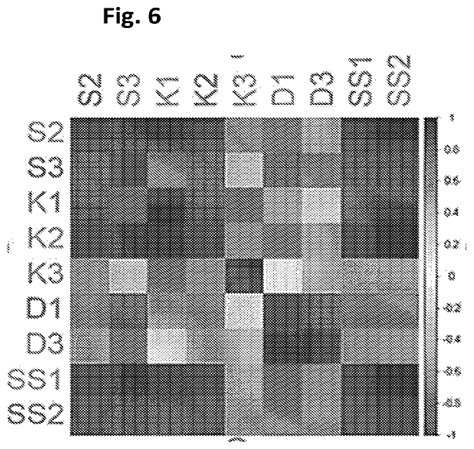

Three target miRNAs (miR-141, miR-142-3p and miR-205) in EBC of three different timepoints of four individuals were detected by realtime PCR and normalized to housekeeping miR-423-3p. It shows the EBC samples from different timepoints of the same subject were stable to a large extent (FIG. 6).

This report entails the most comprehensive interrogation of microRNAs in exhaled breath, here uniquely performed to distinguish subjects with and without primary lung cancers. Starting with a lung tissue microRNA-seq discovery effort, it was interrogated herein a panel of 25 microRNAs in exhaled breath condensate using our RNA-specific qualitative RT-PCR. It was discovered herein that: (i) microRNAs are detectable in exhaled breath condensate; (ii) there are individual exhaled microRNAs that offer case-control discrimination by logistic regression (e.g., microRNAs 21, 33b, 212), and (iii) additional RF models can be developed, using the entire microRNA panel, that also indicate additional case-control discrimination, particularly in the subsets of former smoker, and early stage subjects, over and above that demonstrated in comprehensive clinical models.

Technical challenges abound in examining nucleic acids in EBC. While EBC is widely available non-invasively, this specimen entails only trace levels of microRNA template. This is perhaps because the templates are by definition, higher in molecular weight (20-22 nucleotides in length, >200 carbons) than is typically true for smaller exhaled airstream-suspended molecules (e.g., small polar metabolites), or labile VOC gas-phase molecules, such as H₂O₂, 8-isoprostane, and others. Nonetheless, the PCR confers capacity for detection of microRNAs at the low template copy level, as is suggested herein. The trace concentrations inherent to EBC specimens for most analytes, including nucleic acids has, to date, precluded performing discovery efforts such as microRNA next gen sequencing, directly from this matrix.

The microRNA interrogation panel choice was therefore based on: a previously unpublished microRNA seq effort (GEO #: GSE33858; ENA accession: PRJEB52036) inter-tissue comparison of 32 lung resected bronchogenic carcinoma versus remote lung tissue (stratified for adenocarcinoma, squamous cell carcinoma histologies), with 10 representative overexpressed microRNAs included from each of those two histologies. The remainder came from TCGA, and several literature-identified microRNA markers of lung cancer.

It is used herein microRNA-PCR that is micro/mRNA-specific, as it excludes gDNA fragment false priming by employing a uniquely tagged RT-primer strategy, and in primer design precluded false amplification of messenger RNA fragments. Performance of the fluorescent intercalating (SYBR®) dye detection strategy coupled to URT-PCR on the realtime PCR platform allowed quality assurance using quantitation curve, melt-curve, melt temperature with each PCR reaction. This was superimposed on a series of other analyses invoked during primer design, using multiple positive and negative controls, described in Example 1.

This cross-sectional case-control design was chosen as representing an initial step in early development of potential risk biomarkers. Clinical-demographic differences were observed in cases versus controls for age, smoking, pack-years, quit years, a pack-years minus quit-years composite index, underlying lung disease (COPD, inflammation/fibrosis, asthma, sarcoidosis, bronchiectasis). However, these differences were equally modelled in both clinical-only models and in the clinical+microRNA combined models identically, so they should not have biased the incremental microRNA-attributable risk prediction. Current and former smokers were emphasized, as they are at elevated risk for lung cancer, and therefore commonly come to clinical attention for surveillance, biopsy/resection, and thus were considered appropriately efficient for enrollment in this initial study. The case and control ascertainment presented herein was crisp, minimizing misclassification as subjects were all confirmed histologically by virtue of their bronchoscopic/surgical procedures, underwent further verification of case and control status by an additional 3-6 month period of clinical follow-up, facilitated by electronically-retrieved clinical assessments from the engaged clinical pathologists, radiologists, surgeons, and pulmonologists on each subject. Recruited subjects with disputed case-control ascertainment (<1% of enrolled) were excluded from the study.

In this case-control subject set, with pre-selected candidate 24-microRNA panel, logistic regression was initially performed, using case-control status as the main outcome variable, and a clinical model tested with/without each individual miR on the panel. Separately, iterative cross validation was employed by random forests to assure stability of the results presented herein. The RF approach iteratively and randomly splits the data, substantively cross validating in truly random fashion, and minimizing over-fit.

The clinical versus clinical-microRNA incremental differences are relatively modest (˜0.0-3.0%). This may, in part, be due to the strength of the clinical model alone displaying ROCs ˜0.75-80. These were unusually robust clinical models for two reasons. First is the clinical model comprehensiveness, in part attributable to inclusion of all major known substantive risk factors for lung cancer (including quit years, underlying lung disease, others). Secondly, there is positive selection inherent to enrolling clinical bronchoscopy and surgical subjects such as these (above), wherein both (case and control) sets of subjects are drawn from the same base (procedural-destined) population that is itself selected on clinical criteria to be at high risk for lung cancer. By definition, that high risk is perceived by the clinician as sufficient to warrant an invasive diagnostic/therapeutic procedure, the enrollment point for a majority of our subjects. Both of these factors (clinical model comprehensiveness, and clinical series enrollment bias) contribute to high risk in this clinical series, and imply that clinical risk model performance will be elevated. Thus, the difference between this comprehensive clinical model alone, and that for this clinical model plus microRNA could potentially be narrowed (as compared to that using conventional sparse clinical models) by virtue of the comprehensiveness of the clinical model. The negative impact of such bias on the estimate of the actual contribution of exhaled microRNAs to case-control discrimination, is counter-balanced by the strength inherent in using the same (robust) clinical model when comparing clinical-only models versus combined clinical+microRNA models. Additionally, the definitive diagnoses inherent in recruiting those destined for lung sampling/pathologic readout was another strength. Finally, given that the microRNA PCR was a qualitative assessment (present/absent), such qualitative data often minimize differences, and thereby power to detect such differences. Overall, then, the above considerations suggest that the findings herein are a conservative estimate of the exhaled biomarker contribution in real clinical conditions.

This is a survey of the “state of the epithelium,” i.e., the broad field of early carcinogenesis, rather than detection of a small peripheral tumor itself. This view of broad epithelial “field” interrogation is appropriate to risk assessment, rather than that of a suspect tumor diagnostic tool. That the signal was likely from the field of normal cell material, rather than spillage of a tumor is supported by the observation that early stage subset showed more case-control discrimination than the late stage cases, which would not be expected if the tumor itself was spilling microRNA material.

### Example 7: Lung Tissue-Based Tumor Versus Non-Tumor Discriminant microRNAs Methods for Examples 8-10

### Lung Tissue Subject Recruitment Lung Tissue Handling:

An initial set of 32 consenting individuals destined for surgical lung resection for clinical purposes were approached, consented, enrolled, and contributed surgical resection specimens, both tumor and the marginal non-tumor tissue, as previously described. This accrual entailed enrollment under a protocol approved by the Einstein-Montefiore institutional review board (IRB). Macroscopic specimens were manually divided in the operating room adjacent pathologic cutting, divided into tumor and non-tumor specimens, and snap frozen within 20 minutes of blood supply ligation, as previously described. These tumor and non-tumor pairs from each individual were stored in liquid nitrogen (−170° C.) until use. Approximately 25 mg samples were then homogenized, and total RNA extracted using a TRIzol reagent.

### MiRNA-Sequencing:

MiRNA-seq was performed as recommended by the manufacturer (Illumina small RNA prep kit v 1.5). 10 μg of total RNA from each sample was resolved on a 15% TBE-Urea polyacrylamide gel followed by the excision of gels corresponding to the 17-35 nucleotides. Small RNAs were isolated from the gel in 300 μl of 0.3M NaCl for 4 hours at room temperature. The small RNAs were ligated with a biotinylated RNA-DNA 3′-adaptor, gel-purified, and ligated with a 5′-adaptor. Products with both adaptors were gel-purified, reverse-transcribed, and PCR amplified for 14 cycles. Sequencing was performed on an Illumina GA1 analyzer.

### Data Analysis:

Sequencing results from the Illumina GA2 sequencer are provided in the standardized fastq format reporting base calls certainty of those calls. The files were trimmed of the 3′ adapter sequence. Any reads that did not have a 3′ adapter sequence to trim or contained more than 3 bases with low quality were removed from the analysis through a c++program. To speed up processing time redundant sequences were collapsed and their number of occurrences noted, using the Galaxy Genome Browser tool fastx. All sequences were then aligned to the known miRNA/mRNA database (mirBase) and their corresponding miRNA were determined. To normalize across samples we assumed that the number of annotated miRNA/sequencing reads were the same. After normalization the any reads that were in fewer than 50% of the samples and in copy number less than 10 were not considered for analysis. Then the data was square-root transformed to make it fit a normal distribution so a t-test could be used to determine statistical differences. Multiple testing correction was performed through permutation analysis assuring a false-discovery rate correction of 0.05. All miRNA found to have a FDR corrected p-Value <0.05 were considered to be differentially expressed.

### Example 8: Discovery of miRNAs Expressed in Adenocarcinoma and Squamous Cell Carcinoma of the Lung

Small RNA libraries were generated from normal and tumor tissue of 20 patients with Adenocarcinoma and 10 patients with Squamous Cell Carcinoma. These libraries were sequenced using Illumina GAII sequencer and a total of 502,295,944 and 539,542,457 reads were obtained from Adenocarcinoma tumors and their corresponding normal tissue respectively. A total of 223,208,663 and 222,937,270 reads were obtained from Squamous Cell Carcinoma tumors and their corresponding normal tissue respectively. Low quality reads and those not existing in 50% of their respective samples were removed and the remaining reads were aligned to a database of known miRNAs (World Wide Web at mirbase.org/) (See Example 7 above). We identified a total of 313 miRNA found to have sufficient expression in both samples to be further analyzed. These miRNA were expressed over a wide distribution but in both samples the 10 most abundant miRNA accounted for over 70% of the total miRNA (FIG. 2A and FIG. 2B). The distribution and abundance of miRNA was similar to other miRNA sequencing results using the same technology. A complete list of miRNAs sequenced in each library, total and normalized read counts, and fold differences between tumor and non-tumor tissue is provided below.

### Example 9: Differential Expression of miRNAs Expressed in Adenocarcinoma and Squamous Cell Carcinoma of the Lung

We identified a total of 63 miRNA in Adenocarcinoma that had a multiple testing corrected FDR <0.05 and a fold change >2.0 (Table 4/Tables 5A and 5B). 40 of those were upregulated in Adenocarcinoma and 23 were upregulated in the normal Sample. For the Squamous Cell Carcinoma we found a total of 25 miRNA meeting the same criteria and 19 were upregulated in the tumor and 6 upregulated in the normal tissue (Table 4/Tables 5A and 5B). Six of the miRNAs were identified as upregulated in both tumor types (Table 5, bolded and underlined) hsa-mir-708, hsa-mir-1259, hsa-mir-494, hsa-mir-200a, hsa-miR-21, hsa-miR-135b.

### Example 10: Anti-Correlation of miRNA Expression and Targeted mRNA Expression

MiRNAs exert their effect on the cellular and tissue phenotypes through modulation of transcript expression levels. To fully explore the role of miRNA in driving cancer phenotypes it is necessary to know the targets of differentially expressed miRNA. Current technologies to predict targets use in-silico matching of miRNA seed sequences and mRNA 3′ UTRs. These methods tend to have high false-positive predictions. In order to determine if the miRNA found differentially expressed in our samples are driving change in transcript levels we applied a novel technique to study the anticorrelation of miRNA-mRNA levels. We obtained microarray analysis of transcript levels in our tumor types and determined how many of them were predicted to be targeted by our miRNA.

To determine autocorrelation we generated a predicted target list for each miRNA using the online datasets of TargetScan (World Wide Web at targetscan.org), miRanda (World Wide Web at microrna.org/microrna/home.do). Targets that were predicted using both programs were considered for our analysis. We took the predicted target list of each miRNA and compared this to the mRNA which were found to be differentially expressed with a fold change >1.5 in either Adenocarcinoma or Squamous cell carcinoma. From here generated a list of putative targets that were found to be up-regulated/down-regulated within the tumor samples. A null hypothesis was that the distribution of putative targets would be no different than the background distribution for all the mRNAs. Using a chi-squared test we were able to give a p-value for each miRNA as to its anti-correlation with the differentially expressed transcripts (Tables 6A and 6B).

We found that for our miRNA upregulated in Adenocarcinoma 17 of the 40 miRNA had statistically significant anticorrelation with their predicted targets. For the Squamous cell carcinoma we found that 2 of the 19 miRNA had statistically significant anti-correlation of the predicted targets.

TABLE 4
Summary of Differential Expression of miRNA found in Squamous
Cell Carcinoma and Adenocarcinoma of the lung. (FDR <0.05)

| | | Squamous Cell |
|---|---|---|
| | Adenocarcinoma | Carcinoma |
| Total FDR <0.05: | 128 | 42 |
| Up regulated in Tumor: | 72 | 31 |
| Up regulated in Normal: | 56 | 11 |
| Fold Change >2.0 | 63 | 25 |
| Percent Composition >1% | 9 | 4 |

TABLE 5A
Individuals MicroRNA found to be differentially expressed
in Adenocarcinoma with Fold Change >2.0.

| Up in Adenocarcinoma | | Up in Normal |
|---|---|---|
| hsa-mir-31 | hsa-mir-200b | hsa-mir-516b |
| hsa-mir-147b | hsa-mir-642 | hsa-mir-520f |
| hsa-mir-577 | hsa-mir-625 | hsa-mir-1251 |
| hsa-mir-194 | hsa-mir-146a | hsa-mir-422a |
| hsa-mir-877 | hsa-mir-429 | hsa-mir-486 |
| hsa-mir-96 | hsa-mir-33b | hsa-mir-490 |
| hsa-mir-135b | hsa-mir-21 | hsa-mir-139 |
| hsa-mir-9 | hsa-mir-1248 | hsa-mir-184 |
| hsa-mir-708 | hsa-mir-671 | hsa-mir-516a |
| hsa-mir-556 | hsa-mir-589 | hsa-mir-451 |
| hsa-mir-182 | hsa-mir-193b | hsa-mir-144 |
| hsa-mir-183 | hsa-mir-142 | hsa-mir-126 |
| hsa-mir-210 | hsa-mir-1274b | hsa-mir-30a |
| hsa-mir-1259 | hsa-mir-487a | hsa-mir-195 |
| hsa-mir-1275 | hsa-mir-1226 | hsa-mir-202 |
| hsa-mir-200a | hsa-mir-339 | hsa-mir-585 |
| hsa-mir-301b | hsa-mir-219 | hsa-mir-551b |
| hsa-mir-449c | hsa-mir-29b | hsa-mir-218 |
| hsa-mir-494 | hsa-mir-940 | hsa-mir-133a |
| hsa-mir-375 | hsa-mir-148a | hsa-mir-675 |
| | | hsa-mir-133b |
| | | hsa-mir-598 |
| | | hsa-mir-618 |
| | | hsa-mir-516b |

TABLE 5B
Individuals MicroRNA found to be differentially expressed
in Squamous Cell Carcinoma with Fold Change >2.0.

| Up in Squamous | | Up in Normal |
|---|---|---|
| hsa-mir-708 | hsa-mir-369 | hsa-mir-184 |
| hsa-mir-1259 | hsa-mir-493 | hsa-mir-1251 |
| hsa-mir-494 | hsa-mir-656 | hsa-mir-144 |
| hsa-mir-200a | hsa-mir-301a | hsa-mir-516a |
| hsa-mir-216b | hsa-mir-376a | hsa-mir-584 |
| hsa-mir-1827 | hsa-mir-337 | hsa-mir-519a |
| hsa-mir-431 | hsa-mir-212 | |
| hsa-mir-376b | hsa-mir-376c | |
| hsa-mir-122 | hsa-mir-135b | |
| hsa-mir-21 | | |

TABLE 6A
Anticorrelation of miRNA and their predicted targets in Squamous Cell Carcinoma.

| | | | Predicted | | | | |
|---|---|---|---|---|---|---|---|
| | | | Targets | | | | |
| | | Predicted | Differentially | Targets | Targetd | Chi- | p- |
| miRNA | UP IN: | Targets | Expressed | Correlated | Anticorrelated | squared | Value |
| hsa-miR- | SQUAMOUS | 112 | 9 | 3 | 6 | 0.50 | 0.4795 |
| 708 | | | | | | | |
| hsa-miR- | SQUAMOUS | 145 | 25 | 7 | 18 | 2.42 | 0.1198 |
| 1259 | | | | | | | |
| hsa-miR- | SQUAMOUS | 375 | 38 | 15 | 23 | 0.84 | 0.3588 |
| 494 | | | | | | | |
| hsa-miR- | SQUAMOUS | 580 | 81 | 31 | 50 | 2.23 | 0.1355 |
| 200a | | | | | | | |
| hsa-miR- | SQUAMOUS | 180 | 23 | 4 | 19 | 4.89 | 0.0270 |
| 216b | | | | | | | |
| hsa-miR- | SQUAMOUS | 430 | 52 | 20 | 32 | 1.38 | 0.2393 |
| 1827 | | | | | | | |
| hsa-miR- | SQUAMOUS | 99 | 13 | 7 | 6 | 0.04 | 0.8445 |
| 431 | | | | | | | |
| hsa-miR- | SQUAMOUS | 147 | 26 | 13 | 13 | 0.00 | 1.0000 |
| 376b | | | | | | | |
| hsa-miR- | SQUAMOUS | 130 | 15 | 9 | 6 | 0.30 | 0.5839 |
| 122 | | | | | | | |
| hsa-miR- | SQUAMOUS | 224 | 30 | 12 | 18 | 0.60 | 0.4386 |
| 21 | | | | | | | |
| hsa-miR- | SQUAMOUS | 83 | 6 | 1 | 5 | 1.33 | 0.2482 |
| 493 | | | | | | | |
| hsa-miR- | SQUAMOUS | 596 | 115 | 35 | 80 | 8.80 | 0.0030 |
| 656 | | | | | | | |
| hsa-miR- | SQUAMOUS | 802 | 116 | 49 | 67 | 1.40 | 0.2373 |
| 301a | | | | | | | |
| hsa-miR- | SQUAMOUS | 147 | 26 | 13 | 13 | 0.00 | 1.0000 |
| 376a | | | | | | | |
| hsa-miR- | SQUAMOUS | 293 | 35 | 11 | 24 | 2.41 | 0.1202 |
| 212 | | | | | | | |
| hsa-miR- | SQUAMOUS | 163 | 19 | 6 | 13 | 1.29 | 0.2561 |
| 376c | | | | | | | |
| hsa-miR- | SQUAMOUS | 546 | 65 | 24 | 41 | 2.22 | 0.1360 |
| 135b | | | | | | | |

TABLE 6B
Anticorrelation of miRNA and their predicted targets in Adenocarcinoma.

| | | | Predicted | | | | |
|---|---|---|---|---|---|---|---|
| | | | Targets | | | | |
| | | Predicted | Differentially | Targets | Targetd | Chi- | |
| miRNA | UP IN: | Targets | Expressed | Correlated | Anticorrelated | squared | p-Value |
| hsa-miR- | ADENO | 243 | 26 | 7 | 19 | 2.77 | 0.0961 |
| 31 | | | | | | | |
| hsa-miR- | ADENO | 385 | 49 | 10 | 39 | 8.58 | 0.0034 |
| 577 | | | | | | | |
| hsa-miR- | ADENO | 269 | 34 | 6 | 28 | 7.12 | 0.0076 |
| 194 | | | | | | | |
| hsa-miR- | ADENO | 67 | 6 | 2 | 4 | 0.33 | 0.5637 |
| 877 | | | | | | | |
| hsa-miR- | ADENO | 866 | 105 | 29 | 76 | 10.52 | 0.0012 |
| 96 | | | | | | | |
| hsa-miR- | ADENO | 546 | 62 | 19 | 43 | 4.65 | 0.0311 |
| 135b | | | | | | | |
| hsa-miR- | ADENO | 1037 | 117 | 36 | 81 | 8.65 | 0.0033 |
| 9 | | | | | | | |
| hsa-miR- | ADENO | 112 | 7 | 2 | 5 | 0.64 | 0.4227 |
| 708 | | | | | | | |
| hsa-miR- | ADENO | 927 | 106 | 22 | 84 | 18.13 | 0.0000 |
| 182 | | | | | | | |
| hsa-miR- | ADENO | 309 | 32 | 4 | 28 | 9.00 | 0.0027 |
| 183 | | | | | | | |
| hsa-miR- | ADENO | 145 | 16 | 4 | 12 | 2.00 | 0.1573 |
| 1259 | | | | | | | |
| hsa-miR- | ADENO | 72 | 5 | 1 | 4 | 0.90 | 0.3428 |
| 1275 | | | | | | | |
| hsa-miR- | ADENO | 580 | 80 | 14 | 66 | 16.90 | 0.0000 |
| 200a | | | | | | | |
| hsa-miR- | ADENO | 802 | 98 | 30 | 68 | 7.37 | 0.0066 |
| 301b | | | | | | | |
| hsa-miR- | ADENO | 375 | 42 | 10 | 32 | 5.76 | 0.0164 |
| 494 | | | | | | | |
| hsa-miR- | ADENO | 144 | 11 | 0 | 11 | 5.50 | 0.0190 |
| 375 | | | | | | | |
| hsa-miR- | ADENO | 894 | 126 | 16 | 110 | 35.06 | 0.0000 |
| 200b | | | | | | | |
| hsa-miR- | ADENO | 172 | 17 | 3 | 14 | 3.56 | 0.0592 |
| 642 | | | | | | | |
| hsa-miR- | ADENO | 70 | 7 | 1 | 6 | 1.79 | 0.1814 |
| 625 | | | | | | | |
| hsa-miR- | ADENO | 135 | 23 | 3 | 20 | 6.28 | 0.0122 |
| 146a | | | | | | | |
| hsa-miR- | ADENO | 894 | 126 | 16 | 110 | 35.06 | 0.0000 |
| 429 | | | | | | | |
| hsa-miR- | ADENO | 284 | 38 | 14 | 24 | 1.32 | 0.2513 |
| 33b | | | | | | | |
| hsa-miR- | ADENO | 224 | 32 | 7 | 25 | 5.06 | 0.0244 |
| 21 | | | | | | | |
| hsa-miR- | ADENO | 164 | 14 | 3 | 11 | 2.29 | 0.1306 |
| 1248 | | | | | | | |
| hsa-miR- | ADENO | 116 | 12 | 5 | 7 | 0.17 | 0.6831 |
| 589 | | | | | | | |
| hsa-miR- | ADENO | 144 | 24 | 6 | 18 | 3.00 | 0.0833 |
| 193b | | | | | | | |
| hsa-miR- | ADENO | 133 | 17 | 5 | 12 | 1.44 | 0.2299 |
| 1274b | | | | | | | |
| hsa-miR- | ADENO | 109 | 8 | 1 | 7 | 2.25 | 0.1336 |
| 487a | | | | | | | |
| hsa-miR- | ADENO | 201 | 16 | 6 | 10 | 0.50 | 0.4795 |
| 1226 | | | | | | | |
| hsa-miR- | ADENO | 964 | 115 | 36 | 79 | 8.04 | 0.0046 |
| 29b | | | | | | | |
| hsa-miR- | ADENO | 335 | 35 | 14 | 21 | 0.70 | 0.4028 |
| 940 | | | | | | | |
| hsa-miR- | ADENO | 581 | 74 | 21 | 53 | 6.92 | 0.0085 |
| 148a | | | | | | | |

### Example 11: Developing EBC miRNA Realtime PCR-Methods Used for Examples 12-17

Optimization of EBC microRNA extraction was performed, comparing ethanol alone, ethanol+trizol alone, speed vacuum alone, column-based method alone (FIG. 7) and combinations of the above with glycogen and carrier RNA (FIG. 8). Optimal was ethanol precipitation with glycogen carrier molecule, and the final protocol included an biochemical/Trizol®-based isolation.

### Example 12: Optimization of EBC microRNA Extraction

In FIG. 7, different extraction platforms are tested. Graphs depict quantitative realtime RT-PCR melt curve with SYBR green intercalating dye. Fluorescence is depicted on y-axis, cycle number on x-axis. Ethanol precipitation appeared to be optimal means to extract miRNA from EBC, judging by Ct value (conditions a,b versus c, d)—the lower the Ct value, the earlier the amplified PCR product signal reaches a given threshold fluorescence, implying higher starting template.

### Example 13: Optimization of Nucleic Acid Ethanol Precipitation Conditions (Carriers)

In FIG. 8, carrier molecules were tested, glycogen versus RNA. Quantitative RT-PCR melt curve with SYBR green intercalating dye. Fluorescence is depicted on y-axis, cycle number on x-axis. Ethanol precipitation with either glycogen or RNA carrier performed similarly in extracting miRNA from EBC, judging by Ct value.

### Example 14: Specificity Testing of any Newly Designed microRNA Primer

All microRNA primer designs passed the four specificity tests, viewed by gel electropheresis of final PCR product and key controls (FIG. 9)). These tests included: (a) no-RT step (r/o DNA-derived signal); (b) no polyA-step (r/o messenger RNA-derived signal); (c) genomic DNA spike in (r/o gDNA derived signal); (d) water-no template blank (r/o cDNA or PCR-product contaminated reagents).

FIG. 9 shows how newly designed microRNA primers were tested. For a microRNA-specific URT-PCR primer set, microRNA-size PCR bands (including URT tags ˜60 bp product) are seen in lane 2 (cultured cell template, all polyadenylation and RT steps included), and lane5 (EBC template, all polyadenylation and RT steps included).

### Example 15: Universal RT primer optimization for microRNAs

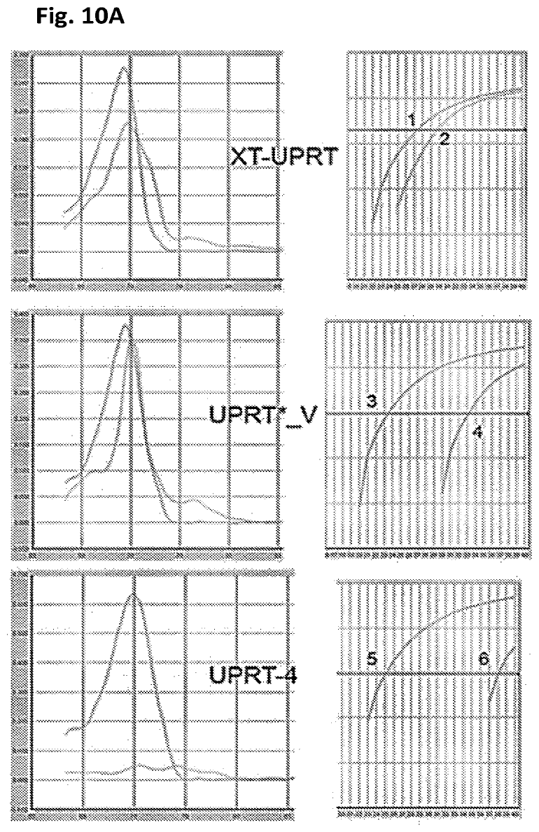

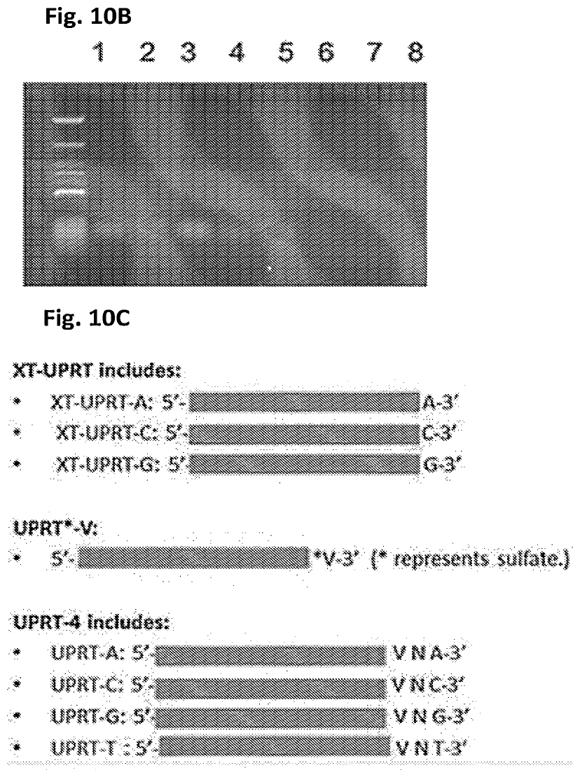

FIG. 10A-FIG. 10C show that different URT designs confer different discrimination of miRNA. Among the options tested, XT-UPRT, UPRT*_V and UPRT-4 were acceptable URT. In this case, miRNA PCR products should not appear in the template of cDNA of mRNA. But in the cDNA of mRNA produced by XT-UPRT and UPRT*_V, miRNA PCR products appeared. Additionally, the melting curves and PCR fragment sizes are very similar between cDNA of miRNA and cDNA of mRNA. For UPRT-4, however, there was no similar melting curve and no PCR band in cDNA of mRNA. Therefore, UPRT-4 was chosen as the best URT.

### Example 16: Distinguishing miRNA from mRNA Based on Different URTs

Table 7 depicts microRNA specificity, expressed as delta CT of mRNA (no polyadenylation step included) versus microRNA product (polyadenylation step included), realtime PCR. The greater the difference (deltaCT), the more specific for microRNA is the microRNA-designed primerset. Melt curve analysis was also analysed (FIG. 10A-FIG. 10C). UPRT*_V and UPRT-4 are adequate URTs. UPRT*_V was selected over UPRT-4. Bolded/underlined highlights acceptable miRNA versus mRNA discrimination, judged by delta-CT.

### Example 17: URT-PCR Sensitivity and Specificity

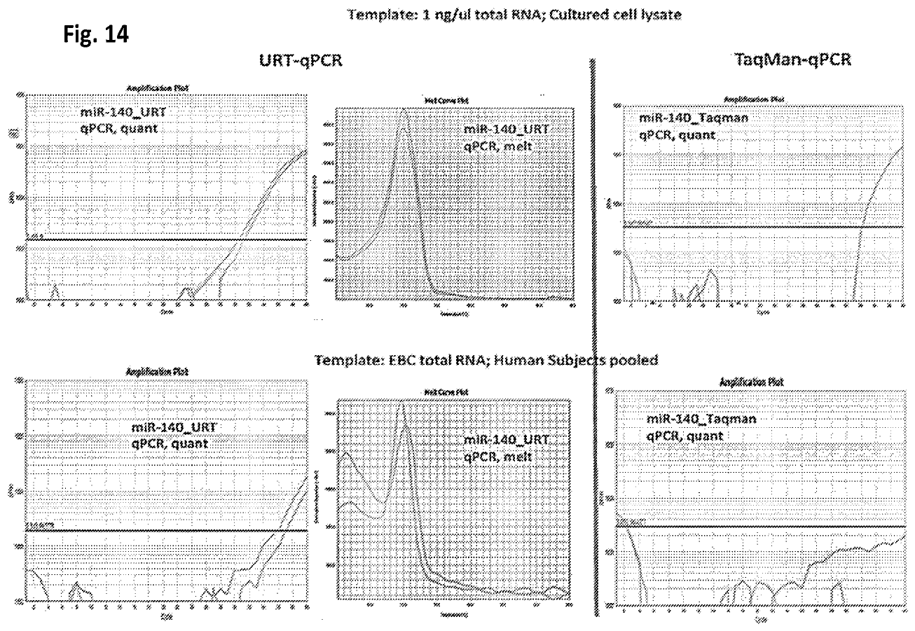

The specificity and sensitivity were compared between URT-qPCR and Taqman qPCR. Positive controls and negative controls, including cDNA of mRNA and miRNA, minus RT, cDNA of mRNA, genomic DNA and water were used to identify an optimal primer pair with microRNA specificity in URT-qPCR. As for specificity of qPCR, both URT-qPCR and Taqman qPCR can be specific for targets (FIG. 14). As for sensitivity of qPCR, URT-qPCR was more sensitive than Taqman qPCR for some low copies templates (let-7a, miR-18a, miR-140, miR-423-3p, miR-708, miR-767 (Table 10).

### Example 18: EBC Surrogacy for the Lung

Table 9 shows a subset of =12 EBC donors providing bronchoscopic samples of deep alveolar (BAL) and major airway (bronchial, BB) levels, as well as exhaled breath (EBC), mouthrinse (MW) sputum (SP) and other specimens. Here, cells that are marked with a “+” sign show qualitative PCR (+) ive for the given miR (columns), offering an miR signature or fingerprint the airway level. So far, pattern recognition suggests that anatomic validation of the origin of the exhaled microRNAs may be, at least in part, derived from the lower airway, as well as from the upper airway.

TABLE 7
Distinguishing miRNA from mRNA based on different URTs.

| miRNA | delta Ct(mRNA- | delta Ct(mRNA- | delta Ct(mRNA- |
|---|---|---|---|
| forward | miRNA) of | miRNA) of | miRNA) of |
| primer | XT-UPRT | UPRT*_V | UPRT-4 |
| 7_q1 | 3 | 10 | 14 |
| 9_N | 3 | 8.5 | 13.5 |
| 18a_q1 | 3.7 | 7.5 | 10 |
| 20a-5p_q2 | 1 | 9 | 9.5 |
| 21_q1 | 2.2 | 5.5 | 1.5 |
| 31_q1 | 5.7 | 12.4 | 14.1 |
| 126_q1 | 4.8 | 13 | 9.5 |
| 130b-3p_q1 | 0.5 | 6 | 5 |
| 135a_q2 | 1.5 | 8 | 3.5 |
| 142-5p_q1 | 4 | 1 | no signal |
| 146a-5p_q3 | 0.5 | 8 | 9 |
| 182-5p_q2 | 2 | 7.5 | 8 |
| 183-5p_q2 | 1.6 | 10 | 4 |
| 191_q1 | 1.2 | 12 | 8 |
| 196a-5p_q3 | 1 | 8.5 | 9 |
| 200a_q1 | 1.8 | 10.5 | 10.5 |
| 200c | 2.2 | 10.2 | 9.7 |
| 205 | 1.2 | 13 | 8 |
| 210_q3 | 3 | 4.3 | 4.3 |
| 212_q1 | 2.2 | 4.2 | 4.2 |
| 221 | 3.6 | 11 | 11.4 |
| 224_q3 | 2.6 | 12.7 | 13.8 |
| 330-3p_q3 | 4 | 1.5 | 2 |
| 708_q3 | 0.7 | 6.7 | 6.7 |

Table 7 depicts microRNA specificity, expressed as delta CT of messenger RNA (no polyadenylation step included) versus microRNA product (polyadenylation step included) by realtime PCR. The greater the difference (deltaCT) between the two conditions, the more microRNA specific is the microRNA primerset.

* The microRNA primers sequences used in the study are as follows in Table 8.

TABLE 8
Primers, EBC-microRNA-PCR

| | | matched |
|---|---|---|
| | | reverse |
| primer name | sequence 5′-3′ | primer |
| miR-9 | CGATCTTTGGTTATCTAGCTGTAT | U60 |
| miR-21 | TAGCTTATCAGACTGAT | U60 |
| miR-31 | AGGCAAGATGCTGGCATAG | U60 |
| miR-33b | GTGCATTGCTGTTGCAT | U60 |
| miR-96 | TTTGGCACTAGCACATT | U60 |
| miR-105 | TCAAATGCTCAGACTCCTGTGG | U60 |
| miR-146a-5p | TGAGAACTGAATTCCATG | UR |
| miR-182-5p | TTTGGCAATGGTAGAACTCAC | U60 |
| miR-196b | TAGGTAGTTTCCTGTTGTTGG | U60 |
| miR-199b-5p | CCAGTGTTTAGACTATCTGTT | U60 |
| miR-200a | TAACACTGTCTGGTAACGATGT | U60 |
| miR-200b | TAATACTGCCTGGTAATGAT | U60 |
| miR-205 | CTTCATTCCACCGGAGTCT | U60 |
| miR-212 | TAACAGTCTCCAGTCACGGC | U60 |
| miR-221 | GCTACATTGTCTGCTGGGTT | U60 |
| miR-324-5p | GCATCCCCTAGGGCATTGG | U60 |
| miR-345 | GCTGACTCCTAGTCCAGGGC | U60 |
| miR-423-3p | CTCGGTCTGAGGCCCCTC | U60 |
| miR-429 | TAATACTGTCTGGTAAAACCG | U60 |
| miR-767 | TGCACCATGGTTGTCTGAGC | U60 |
| miR-944 | AAATTATTGTACATCGGATG | UR |
| miR-1269a | CTGGACTGAGCCGTGCTACTG | U60 |
| miR-1293 | TGGGTGGTCTGGAGATTTGT | U60 |
| miR-1910 | CCAGTCCTGTGCCTGCCGC | U60 |
| miR-3662 | GAAAATGATGAGTAGTGACTGAT | U60 |
| URT | AACGAGACGACGACAGACTTTTTT | |
| U60 | AACGAGACGACGACAGACTTT | |
| UR | AACGAGACGACGACAGAC | |

TABLE 9 Airway level of origin patterns: EBC, MW, SP, BB, BAL; Summary table

TABLE 9
EBC surrogacy for the lung: A subset of =12 EBC donors providing bronchoscopic samples of deep
alveolar (BAL) and major airway (bronchial, BB) levels, as well as exhaled breath (EBC), mouthrinse
(MW), sputum (SP) and other specimens. Here, cells with the “+” mark show qualitative
PCR (+) for the given miR (columns), offering a miR signature or fingerprint of the airway level.

| EBCSubject | | | | | | | | | | | | | | |
|---|---|---|---|---|---|---|---|---|---|---|---|---|---|---|
| Patient# | miR- | | | | | | | | | | | | | |
| (EBC) | 7 | 9 | 18a | 21 | 31 | 126 | 191 | 200c | 205 | 210 | 212 | 221 | 224 | 708 |
| 565 | − | + | − | − | − | − | − | − | − | + | + | + | − | − |
| 573 | − | − | − | − | − | − | − | + | − | + | + | − | + | − |
| 594 | + | − | − | − | − | − | − | − | − | − | + | − | − | − |
| 601 | + | − | + | − | − | − | − | − | − | − | + | + | − | − |
| 614 | + | − | − | − | − | − | − | − | − | + | + | − | − | + |
| 615 | − | + | + | − | − | − | − | − | − | + | + | − | − | − |
| 635 | − | − | − | − | + | + | − | + | + | + | + | + | + | − |
| 636 | − | − | + | − | − | − | − | + | + | + | + | + | + | + |
| 651 | − | + | + | − | + | − | + | − | + | − | + | − | + | − |
| 671 | − | − | − | − | + | − | − | + | − | + | − | + | − | − |
| 706 | + | − | − | − | + | − | + | + | + | + | + | + | − | + |
| 708 | − | − | − | − | + | − | − | − | + | + | − | + | − | − |

| | 7 | 9 | 18a | 21 | 31 | 126 | 191 | 200c | 205 | 210 | 212 | 221 | 224 | 708 |
|---|---|---|---|---|---|---|---|---|---|---|---|---|---|---|
| MWSubject | | | | | | | | | | | | | | |
| (MW) | | | | | | | | | | | | | | |
| 565 | NA | NA | NA | NA | NA | NA | NA | NA | NA | NA | NA | NA | NA | NA |
| 573 | − | − | − | − | + | − | − | + | − | + | − | − | − | − |
| 594 | + | + | − | − | + | − | − | + | − | + | + | + | + | − |
| 601 | + | + | + | − | + | − | + | + | − | + | + | + | − | + |
| 614 | + | + | + | − | + | − | − | + | − | + | + | + | + | + |
| 615 | + | + | + | − | + | − | + | + | + | + | + | + | + | + |
| 635 | + | + | + | − | + | − | + | + | + | + | + | + | + | + |
| 636 | + | + | + | − | + | − | + | + | + | + | + | + | + | + |
| 651 | + | + | + | − | + | + | + | − | + | + | − | + | − | + |
| 671 | + | + | + | − | + | − | + | + | + | + | + | + | + | + |
| 706 | + | + | + | − | + | − | + | + | + | + | + | + | + | + |
| 708 | + | + | + | − | + | − | − | + | + | + | + | + | + | + |
| SPSubject | | | | | | | | | | | | | | |
| (SPUTUM) | | | | | | | | | | | | | | |
| 565 | NA | NA | NA | NA | NA | NA | NA | NA | NA | NA | NA | NA | NA | NA |
| 573 | + | − | + | − | + | − | + | + | − | − | + | + | − | − |
| 594 | − | − | − | − | − | − | − | + | − | − | − | + | − | − |
| 610 | − | − | − | − | − | − | − | − | − | − | − | − | − | − |
| 614 | + | − | − | − | − | − | − | + | − | − | − | + | − | − |
| 615 | − | − | − | − | − | − | − | + | − | − | − | + | − | − |
| 635 | + | + | + | − | + | − | + | + | + | + | + | + | + | + |
| 636 | + | + | + | − | + | − | − | + | + | + | + | + | + | − |
| 651 | + | + | + | − | + | − | + | + | + | + | + | + | + | + |
| 671 | + | + | + | − | + | − | + | + | + | + | − | + | + | + |
| 706 | + | + | + | − | − | − | − | + | + | − | − | + | + | + |
| 708 | + | + | + | − | + | − | − | + | + | + | + | + | + | + |
| BBSubject | | | | | | | | | | | | | | |
| (BB) | | | | | | | | | | | | | | |
| 565 | − | − | − | − | − | − | − | − | − | − | − | − | − | − |
| 573 | + | + | + | + | + | + | + | + | − | + | − | + | + | + |
| 594 | + | + | + | + | + | − | + | + | + | + | + | + | + | + |
| 601 | + | + | + | + | + | + | + | + | + | + | + | + | + | + |
| 614 | − | − | − | − | − | − | − | − | − | − | − | − | − | − |
| 615 | + | + | + | − | + | + | + | + | − | + | − | + | + | + |
| 635 | + | + | + | + | + | + | + | + | + | + | + | + | + | + |
| 636 | + | + | + | + | + | + | + | + | + | + | + | + | + | + |
| 651 | + | + | + | + | + | + | + | + | + | + | + | + | + | + |
| 671 | + | + | + | + | + | + | + | + | + | + | + | + | + | + |
| 706 | + | + | + | + | + | + | + | + | + | + | + | + | + | + |
| 708 | + | + | + | + | + | + | + | + | + | + | + | + | + | + |
| BALSubject | | | | | | | | | | | | | | |
| (BAL) | | | | | | | | | | | | | | |
| 565 | + | + | + | + | + | + | + | + | − | + | + | + | + | + |
| 573 | + | + | + | + | + | + | + | + | − | + | + | + | + | + |
| 594 | − | − | − | − | − | − | − | − | − | − | − | − | − | − |
| 601 | + | + | + | − | + | − | + | + | − | + | + | + | + | + |
| 614 | + | + | + | + | + | + | + | + | − | + | − | + | + | + |
| 615 | + | + | + | + | + | + | + | + | − | + | + | + | + | + |
| 635 | + | + | + | − | + | + | − | + | + | + | + | + | + | + |
| 636 | + | + | + | − | + | − | + | + | + | + | + | − | + | + |
| 651 | + | + | + | − | + | + | + | + | + | + | + | + | + | + |
| 671 | + | + | + | − | + | + | + | + | + | + | + | + | + | + |
| 706 | + | + | + | + | + | + | + | + | + | + | + | + | + | + |
| 708 | + | + | + | − | + | + | + | + | + | + | + | + | + | + |

TABLE 10
URT-qPCR versus Taqman qPCR. Numbers represent Ct values.

| | URT-qPCR | | | Taqman qPCR | | |
|---|---|---|---|---|---|---|
| | 100 | 1 | | 100 | 1 | |
| Template | ng/ul | ng/ul | | ng/ul | ng/ul | |
| average Ct | total | total | EBC | total | total | EBC |
| targets | RNA | RNA | miRNAs | RNA | RNA | miRNAs |
| let-7a | 17.15 | 24.03 | 36.60* | 19.53 | 27.99 | ND |
| let-7f | 24.64 | 30.48 | ND | 22.78 | 31.76 | ND |
| miR-18a | 25.3 | 27.38 | 33.46* | 21.3 | 31.03 | ND |
| miR-21 | 26.93 | 32.6 | ND | 20.44 | 31.29 | ND |
| miR-26a | 21.93 | 27.93 | ND | 20.14 | 29.6 | 33.95 |
| miR-140 | 30.29 | 31.37 | 36.53* | 25.44 | 34.19 | ND |
| miR-212 | 21.66 | 20.3 | 31.51 | 29.28 | 37.11 | 32.75 |
| miR-423-3p | 19.87 | 25.63 | 32.28* | ND | ND | ND |
| miR-708 | 24.75 | 28.28 | 37.43* | 21.87 | 30.84 | ND |
| miR-767 | 16.16 | 21.72 | 34.88* | ND | ND | ND |

ND = not detected,
*Detected in URT, but not detected in the TaqMan platform.

### LIST OF ABBREVIATIONS

TCGA: The Cancer Genome Anatomy project. NCBI/NCI/NIH.
AUC: Area under curve, for receiver operating curve (ROC).
ROC: Test performance plots sensitivity versus specificity.

### Data Availability

The microRNA-seq datasets generated and/or analyzed herein are available in the ENA depository [PRJEB52036].

### REFERENCES

1. Gould M K: Clinical practice. Lung-cancer screening with low-dose computed tomography. N Engl J Med 2014, 371 (19): 1813-1820.
2. Bach P B, Mirkin J N, Oliver T K, Azzoli C G, Berry D A, Brawley O W, Byers T, Colditz G A, Gould M K, Jett J R et al: Benefits and harms of C T screening for lung cancer: a systematic review. JAMA 2012, 307 (22): 2418-2429.
3. Tan B B, Flaherty K R, Kazerooni E A, Iannettoni M D: The solitary pulmonary nodule. Chest 2003, 123 (1 Suppl): 89S-96S.
4. Gould M K, Donington J, Lynch W R, Mazzone P J, Midthun D E, Naidich D P, Wiener R S: Evaluation of individuals with pulmonary nodules: when is it lung cancer? Diagnosis and management of lung cancer, 3rd ed: American College of Chest Physicians evidence-based clinical practice guidelines. Chest 2013, 143 (5 Suppl): e93S-e120S.
5. Mazzone P J, Sears C R, Arenberg D A, Gaga M, Gould M K, Massion P P, Nair V S, Powell C A, Silvestri G A, Vachani A et al: Evaluating Molecular Biomarkers for the Early Detection of Lung Cancer: When Is a Biomarker Ready for Clinical Use? An Official American Thoracic Society Policy Statement. Am J Respir Crit Care Med 2017, 196 (7): e15-e29.
6. Raji O Y, Duffy S W, Agbaje O F, Baker S G, Christiani D C, Cassidy A, Field J K: Predictive accuracy of the Liverpool Lung Project risk model for stratifying patients for computed tomography screening for lung cancer: a case-control and cohort validation study. Ann Intern Med 2012, 157 (4): 242-250.
7. D'Amelio A M, Jr., Cassidy A, Asomaning K, Raji O Y, Duffy S W, Field J K, Spitz M R, Christiani D, Etzel C J: Comparison of discriminatory power and accuracy of three lung cancer risk models. Br J Cancer 2010, 103 (3): 423-429.
8. Tanoue L T, Tanner N T, Gould M K, Silvestri G A: Lung cancer screening. Am J Respir Crit Care Med 2015, 191 (1): 19-33.
9. Kanodra N M, Silvestri G A, Tanner N T: Screening and early detection efforts in lung cancer. Cancer 2015, 121 (9): 1347-1356.
10. Nadal E, Truini A, Nakata A, Lin J, Reddy R M, Chang A C, Ramnath N, Gotoh N, Beer D G, Chen G: A Novel Serum 4-microRNA Signature for Lung Cancer Detection. Sci Rep 2015, 5:12464.
11. Wozniak M B, Scelo G, Muller D C, Mukeria A, Zaridze D, Brennan P: Circulating MicroRNAs as Non-Invasive Biomarkers for Early Detection of Non-Small-Cell Lung Cancer. PLOS One 2015, 10 (5): e0125026.
12. Silvestri G A, Vachani A, Whitney D, Elashoff M, Porta Smith K, Ferguson J S, Parsons E, Mitra N, Brody J, Lenburg M E et al: A Bronchial Genomic Classifier for the Diagnostic Evaluation of Lung Cancer. N Engl J Med 2015, 373 (3): 243-251.
13. Spira A, Beane J E, Shah V, Steiling K, Liu G, Schembri F, Gilman S, Dumas Y M, Calner P, Sebastiani P et al: Airway epithelial gene expression in the diagnostic evaluation of smokers with suspect lung cancer. Nat Med 2007, 13 (3): 361-366.
14. Pavel A B, Campbell J D, Liu G, Elashoff D, Dubinett S, Smith K, Whitney D, Lenburg M E, Spira A: Alterations in Bronchial Airway miRNA Expression for Lung Cancer Detection. Cancer Prev Res (Phila) 2017, 10 (11): 651-659.
15. Comprehensive molecular profiling of lung adenocarcinoma. Nature 2014, 511 (7511): 543-550.
16. Comprehensive genomic characterization of squamous cell lung cancers. Nature 2012, 489 (7417): 519-525.
17. Liu Z L, Wang H, Liu J, Wang Z X: MicroRNA-21 (miR-21) expression promotes growth, metastasis, and chemo- or radioresistance in non-small cell lung cancer cells by targeting PTEN. Mol Cell Biochem 2013, 372 (1-2): 35-45.
18. Liu X, Sempere L F, Ouyang H, Memoli V A, Andrew A S, Luo Y, Demidenko E, Korc M, Shi W, Preis M et al: MicroRNA-31 functions as an oncogenic microRNA in mouse and human lung cancer cells by repressing specific tumor suppressors. J Clin Invest 2010, 120 (4): 1298-1309.
19. Li Y, Zhang D, Chen C, Ruan Z, Huang Y: MicroRNA-212 displays tumor-promoting properties in non-small cell lung cancer cells and targets the hedgehog pathway receptor PTCH1. Mol Biol Cell 2012, 23 (8): 1423-1434.
20. Garofalo M, Di Leva G, Romano G, Nuovo G, Suh S S, Ngankeu A, Taccioli C, Pichiorri F, Alder H, Secchiero P et al: miR-221&222 regulate TRAIL resistance and enhance tumorigenicity through PTEN and TIMP3 downregulation. Cancer Cell 2009, 16 (6): 498-509.
21. Vandenbroucke J P, von Elm E, Altman D G, Gotzsche P C, Mulrow C D, Pocock S J, Poole C, Schlesselman J J, Egger M: Strengthening the Reporting of Observational Studies in Epidemiology (STROBE): explanation and elaboration. Epidemiology 2007, 18 (6): 805-835.
22. Horvath I, Hunt J, Barnes P J, Alving K, Antczak A, Baraldi E, Becher G, van Beurden W J, Corradi M, Dekhuijzen R et al: Exhaled breath condensate: methodological recommendations and unresolved questions. Eur Respir J 2005, 26 (3): 523-548.
23. Hurteau G J, Spivack S D: mRNA-specific reverse transcription-polymerase chain reaction from human tissue extracts. Anal Biochem 2002, 307 (2): 304-315.
24 Hurteau G J, Carlson J A, Spivack S D, Brock G J: Overexpression of the microRNA hsa-miR-200c leads to reduced expression of transcription factor 8 and increased expression of E-cadherin. Cancer Res 2007, 67 (17): 7972-7976.
25. Hurteau G J, Spivack S D, Brock G J: Potential mRNA degradation targets of hsa-miR-200c, identified using informatics and qRT-PCR. Cell Cycle 2006, 5 (17): 1951-1956.
26. Babion I, Snoek B C, van de Wiel M A, Wilting S M, Steenbergen RDM: A Strategy to Find Suitable Reference Genes for miRNA Quantitative PCR Analysis and Its Application to Cervical Specimens. J Mol Diagn 2017, 19 (5): 625-637.
27. Link F, Krohn K, Schumann J: Identification of stably expressed housekeeping miRNAs in endothelial cells and macrophages in an inflammatory setting. Sci Rep 2019, 9 (1): 12786.
28. Lange T, Stracke S, Rettig R, Lendeckel U, Kuhn J, Schluter R, Rippe V, Endlich K, Endlich N: Identification of miR-16 as an endogenous reference gene for the normalization of urinary exosomal miRNA expression data from CKD patients. PLOS One 2017, 12 (8): e0183435.
29. Torres A, Torres K, Wdowiak P, Paszkowski T, Maciejewski R: Selection and validation of endogenous controls for microRNA expression studies in endometrioid endometrial cancer tissues. Gynecol Oncol 2013, 130 (3): 588-594.
30. Sauer E, Babion I, Madea B, Courts C: An evidence based strategy for normalization of quantitative PCR data from miRNA expression analysis in forensic organ tissue identification. Forensic Sci Int Genet 2014, 13:217-223.
31. Perez-Sanchez C, Barbarroja N, Pantaleao L C, Lopez-Sanchez L M, Ozanne S E, Jurado-Gamez B, Aranda E, Lopez-Pedrera C, Rodriguez-Ariza A: Clinical Utility of microRNAs in Exhaled Breath Condensate as Biomarkers for Lung Cancer. Journal of Personalized Medicine 2021, 11 (2).
32. Breiman L: Random forests. Machine Learning 2001, 45 (1): 5-32.
33. Mozzoni P, Banda I, Goldoni M, Corradi M, Tiseo M, Acampa O, Balestra V, Ampollini L, Casalini A, Carbognani P et al: Plasma and EBC microRNAs as early biomarkers of non-small-cell lung cancer. Biomarkers 2013, 18 (8): 679-686.
34. Pepe M S, Etzioni R, Feng Z, Potter J D, Thompson M L, Thornquist M, Winget M, Yasui Y: Phases of biomarker development for early detection of cancer. J Natl Cancer Inst 2001, 93 (14): 1054-1061.
35. Pepe M S, Feng Z, Janes H, Bossuyt P M, Potter J D: Pivotal evaluation of the accuracy of a biomarker used for classification or prediction: standards for study design. J Natl Cancer Inst 2008, 100 (20): 1432-1438.
36. Lin J, Marquardt G, Mullapudi N, Wang T, Han W, Shi M, Keller S, Zhu C, Locker J, Spivack S D: Lung cancer transcriptomes refined with laser capture microdissection. Am J Pathol 2014, 184 (11): 2868-2884.
37. Mullapudi N, Ye B, Suzuki M, Fazzari M, Han W, Shi M K, Marquardt G, Lin J, Wang T, Keller S et al: Genome Wide Methylome Alterations in Lung Cancer. PLOS One 2015, 10 (12): e0143826.
38. Cock P J, Fields C J, Goto N, Heuer M L, Rice P M: The Sanger FASTQ file format for sequences with quality scores, and the Solexa/Illumina FASTQ variants. Nucleic Acids Res 2009.
39. Blankenberg D, Von Kuster G, Coraor N, Ananda G, Lazarus R, Mangan M, Nekrutenko A, Taylor J: Galaxy: a web-based genome analysis tool for experimentalists. Curr Protoc Mol Biol 2010, Chapter 19: Unit 19 10 11-21.
40. Creighton C J, Reid J G, Gunaratne P H: Expression profiling of microRNAs by deep sequencing. Brief Bioinform 2009, 10 (5): 490-497.
41. Morin R D, O'Connor M D, Griffith M, Kuchenbauer F, Delaney A, Prabhu A L, Zhao Y, McDonald H, Zeng T, Hirst M et al: Application of massively parallel sequencing to microRNA profiling and discovery in human embryonic stem cells. Genome Res 2008, 18 (4): 610-621.
42. Bar M, Wyman S K, Fritz B R, Qi J, Garg K S, Parkin R K, Kroh E M, Bendoraite A, Mitchell P S, Nelson A M et al: MicroRNA discovery and profiling in human embryonic stem cells by deep sequencing of small RNA libraries. Stem Cells 2008, 26 (10): 2496-2505.
43. Lewis B P, Burge C B, Bartel D P: Conserved seed pairing, often flanked by adenosines, indicates that thousands of human genes are microRNA targets. Cell 2005, 120 (1): 15-20.
44. Grimson A, Farh K K, Johnston W K, Garrett-Engele P, Lim L P, Bartel D P: MicroRNA targeting specificity in mammals: determinants beyond seed pairing. Mol Cell 2007, 27 (1): 91-105.
45. John B, Enright A J, Aravin A, Tuschl T, Sander C, Marks D S: Human microRNA targets (vol 2, pg 1862, 2005). Plos Biology 2005, 3 (7): 1328-1328.
46. Mitchell et al. ((2021) J Extracell Vesicles 10: e12110

### INCORPORATION BY REFERENCE

The contents of all references, patent applications, patents, and published patent applications, as well as the Figures and the Sequence Listing, cited throughout this application are hereby incorporated by reference.

### EQUIVALENTS

Those skilled in the art will recognize, or be able to ascertain using no more than routine experimentation, many equivalents to the specific embodiments of the present invention described herein. Such equivalents are intended to be encompassed by the following claims.

What is claimed is:

1. A method of detecting an exhaled microRNA (miRNA) of a subject, the method comprising:
(a) collecting exhaled breath condensate (EBC); and
(b) detecting the presence and/or level of the exhaled miRNA,
optionally wherein the exhaled miRNA is selected from the miRNA listed in Table 2, Table 5A, Table 5B, and FIG. 2, or any combination of two or more thereof.

2. The method of claim 1, wherein the exhaled miRNA comprises hsa-mir-101, hsa-mir-451, hsa-mir-148a, hsa-mir-142, hsa-mir-146b, hsa-mir-24, hsa-let-7f, hsa-let-7a, hsa-mir-143, hsa-mir-21, hsa-mir-708, hsa-mir-1259, hsa-mir-494, hsa-mir-200a, hsa-miR-135b, miRNA 33b, miRNA 212, or any combination of two or more thereof.

3. The method of claim 1 or 2, wherein the exhaled miRNA comprises miRNA 21, miRNA 33b, miRNA 212, or any combination of two or more thereof.

4. The method of any one of claims 1-3, wherein the EBC is collected by condensing the exhaled breath in a cooling chamber.

5. The method of any one of claims 1-4, wherein the total EBC is used for detecting the presence and/or level of the exhaled miRNA.

6. The method of any one of claims 1-5, wherein the EBC is partitioned to isolate an exosomal fraction (e.g., small EVs) prior to detecting the presence and/or level of the exhaled miRNA.

7. The method of claim 6, wherein the EBC is partitioned into an exosomal fraction using EV-CATCHER.

8. The method of any one of claims 1-7, wherein the exhaled miRNA detected comprises an exosomal miRNA and/or a non-exosomal miRNA.

9. The method of any one of claims 1-8, wherein the presence and/or level of the exhaled miRNA is detected by a method comprising: RNA-seq, next generation sequencing, sequencing, mass spectrometry (e.g., RNA sequencing by LC-MS, DNA sequencing by LC-MS), microarray, Northern blotting, reverse transcription, Southern blotting, RT-PCR, miRNA PCR, PCR (e.g., URT-PCR), realtime PCR (e.g., TaqMan®), testing for a differential melting temperature of a complementary DNA (cDNA) duplex of miRNA, any variation thereof, or any combination of two or more thereof.

10. The method of any one of claims 1-9, wherein the method further comprises poly-A-tailing the miRNA before detection.

11. The method of any one of claims 1-10, wherein the method further comprises reverse transcribing the miRNA into a cDNA, before detection.

12. The method of any one of claims 1-11, wherein the method comprises miRNA that is poly-A-tailed and PCR-amplified using a primer specific to the poly-A (e.g., those comprising an oligo-dT sequence) and a miRNA-specific primer, before detection.

13. The method of any one of claims 1-12, wherein the level of miRNA is normalized to a housekeeping miRNA present in the EBC.

14. The method of claim 13, wherein the housekeeping miRNA is hsa-miR-16, hsa-miR-26b, hsa-miR-92, hsa-miR-423, hsa-miR-374, or miR-423-3p, miR-21A, miR let-7a, miR let-7f.

15. A method of diagnosing a lung disease, or risk for a lung disease, in a subject, the method comprising:
(a) detecting the presence and/or level of the exhaled miRNA according to the method of any one of claims 1-14; and
(b) comparing said presence and/or level of the exhaled miRNA to a control,
wherein the presence and/or significantly higher level of the exhaled miRNA as compared to the control indicates that the subject has a lung disease.

16. A method of recurrence of a lung disease in a subject, the method comprising:
(a) obtaining a subject sample (e.g., EBC or any derivative thereof) from the subject who has received a therapy for the lung disease;
(b) detecting the presence and/or level of the exhaled miRNA according to the method of any one of claims 1-14; and
(c) comparing said presence and/or level of the exhaled miRNA to a control,
wherein the presence and/or significantly higher level of the exhaled miRNA as compared to the control indicates a recurrence a lung disease in the subject.

17. A method of monitoring the progression of a lung disease in a subject, the method comprising:
(a) detecting in a subject sample (e.g., EBC or any derivative thereof) at a first point in time the presence and/or level of the exhaled miRNA according to the method of any one of claims 1-14;
(b) repeating step (a) at a subsequent point in time; and
(c) comparing the presence and/or level of the exhaled miRNA detected in steps (a) and (b) to monitor the progression of the lung disease in the subject.

18. The method of claim 17, wherein between the first point in time and the subsequent point in time, the subject has received a therapy that treats the disease (e.g., cancer therapy).

19. A method of assessing the efficacy of a therapy that treats a lung disease in a subject, the method comprising:
(a) determining the presence and/or level of the exhaled miRNA according to the method of any one of claims 1-14;
(b) repeating step (a) during at least one subsequent point in time after administration of the therapy; and
(c) comparing the presence and/or level of the exhaled miRNA detected in steps (a) and (b),
wherein a significantly lower level of the exhaled miRNA in the at least one subsequent sample, relative to the first sample, is an indication that the therapy is efficacious to the lung disease in the subject.

20. The method of any one of claims 17-19, wherein the first and/or at least one subsequent sample is a portion of a single sample or pooled samples obtained from the subject.

21. The method of any one of claims 15-18, wherein said significantly higher level of the exhaled miRNA comprises an at least 20% increase in the level of the exhaled miRNA, optionally at least 50% increase in the level of the exhaled miRNA.

22. The method of claim 19, wherein said significantly lower level of the exhaled miRNA comprises an at least 20% decrease in the level of the exhaled miRNA, optionally at least 50% decrease in the level of the exhaled miRNA.

23. The method of claim 15 or 16, wherein the control is a miRNA level in the EBC or a derivative thereof from a healthy subject.

24. The method of any one of claims 15-23, further comprising recommending, prescribing, and/or administering to the subject a therapy (e.g., a cancer therapy).

25. A method of preventing or treating a disease in a subject, the method comprising administering to the subject a therapy, wherein the subject is determined to be in need of a therapy according to the method of any one of claims 14-22.

26. The method of any one of claims 1-24, wherein the subject is healthy, the subject is afflicted with a lung disease, or the subject is at risk for developing a lung disease.

27. The method of any one of claims 15-26, wherein the lung disease is a cancer (e.g., an early lung cancer), asthma, or chronic obstructive pulmonary disease (COPD).

28. The method of any one of claims 16, 18, 19, 24, and 25, wherein the therapy treats a cancer, asthma, or COPD.

29. The method of claim 27 or 28, wherein the cancer is adenocarcinoma, squamous cell carcinoma, small cell lung cancer (SCLC), non-small cell lung cancer (NSCLC), or metastases from another organ to a lung.

30. The method of claim 28, wherein the therapy comprises a surgery, chemotherapy, cancer vaccines, chimeric antigen receptors, radiation therapy, immunotherapy, a modulator of expression of immune checkpoint inhibitory proteins or ligands, corticosteroids, leukotriene modifiers, combination inhalers, theophylline, beta agonists, anticholinergic agents, bronchodilators, steroids, phosphodiesterase-4 inhibitors, antibiotics, or any combination thereof.

31. The method of any one of claims 1-30, wherein the subject is a mammal.

32. The method of any one of claims 1-31, wherein the subject is a mouse or a human.

33. The method of any one of claims 1-32, wherein the subject is a never smoker, former smoker, or a current smoker.

34. The method of any one of claims 1-33, wherein the subject has an underlying lung disease, optionally wherein the underlying lung disease is selected from chronic obstructive pulmonary disease (COPD), fibrosis, inflammation, asthma, sarcoidosis, and bronchiectasis.
